(19) United States
(12) Patent Application Publication
(10) Pub. No.: US 2023/0317292 A1
(43) Pub. Date: Oct. 5, 2023

(54) METHODS AND SYSTEMS FOR OPTIMIZING OF SENSOR WEAR AND/OR LONGEVITY OF A PERSONALIZED MODEL USED FOR ESTIMATING GLUCOSE VALUES

(71) Applicant: MEDTRONIC MINIMED, INC., Northridge, CA (US)

(72) Inventors: Arthur Mikhno, Princeton, NJ (US); Yuxiang Zhong, Arcadia, CA (US); Pratik J. Agrawal, Porter Ranch, CA (US)

(21) Appl. No.: 17/695,801

(22) Filed: Mar. 15, 2022

(51) Int. Cl. G16H 50/30; A61B 5/145; G06F 30/27

(52) U.S. Cl. CPC G16H 50/30; A61B 5/14532; G06F 30/27; G06F 2119/02

(57) ABSTRACT

Methods, systems and non-transient computer-readable media are provided for optimizing sensor wear and/or longevity of a personalized model used for estimating glucose values.

### TECHNICAL FIELD

The present technology is generally related to insulin delivery systems and, more specifically, to a machine learning-based system for estimating glucose values based on, for example, blood glucose measurements and contextual activity data. Some of the disclosed embodiments relate to technologies for optimizing of sensor wear and/or longevity of a personalized model used for estimating glucose values.

### BACKGROUND

Portable medical devices are useful for patients that have conditions that must be monitored on a continuous or frequent basis. For example, diabetics are usually required to modify and monitor their daily lifestyle to keep their blood glucose (BG) in balance. Individuals with Type 1 diabetes and some individuals with Type 2 diabetes use insulin (and other blood sugar lowering medications) to control their BG levels. To maintain glucose levels within a recommended range, patients with diabetes are advised to routinely keep strict schedules, including ingesting timely nutritious meals, partaking in exercise, monitoring BG levels daily, and adjusting and administering insulin dosages accordingly.

Infusion pump devices and insulin pump systems are relatively well known in the medical arts. Infusion pump devices and insulin systems are designed to deliver accurate and measured doses of insulin via infusion sets. An infusion set delivers the insulin to a patient through a small diameter tube that terminates at, e.g., a cannula inserted under the patient’s skin. Use of infusion pump therapy has been increasing, especially for delivering insulin for diabetics.

In one type of system, the infusion pump can be programmed to inject insulin on a set schedule. For instance, a doctor or other healthcare administrator can program an insulin pump according to a set schedule so that it delivers insulin into the patient’s bloodstream according to that schedule throughout the day. In addition, the patient can also simply activate the insulin pump to administer an insulin bolus as needed, for example, in response to the patient’s high BG level. One drawback to this approach is that the user’s lack knowledge of their blood glucose levels in any given point, and it is difficult for patients to determine when it may be necessary to administer insulin outside their scheduled administrations.

To address this problem, a patient can monitor BG levels using a BG meter or measurement device. For instance, a patient can utilize a blood glucose meter to intermittently measure their instantaneous blood sugar levels at any given time via a “finger prick test.” Information from that measurement can be processed and used to determine whether insulin should be administered to regulate the patient’s blood sugar level. A problem with this approach is that monitoring of blood glucose levels and the administration of insulin is done by the user on an ad hoc basis based on when they think insulin should be administered. This can be problematic in certain situations, such as with patients who experience hypoglycemia unawareness, which is a condition that can occur where a patient cannot perceive symptoms that might be indicators that their blood sugar is low.

To address this issue, continuous glucose monitoring (CGM) systems can be used to help diabetic patients continuously monitor their blood glucose levels. Continuous glucose monitoring systems employ a continuous glucose sensor to monitor a patient’s blood glucose level in a substantially continuous and autonomous manner. In many cases, a continuous glucose monitoring sensor is utilized in conjunction with an insulin infusion device or insulin pump as parts of a digital diabetes management system. The digital diabetes management system can determine the amount of glucose in the patient’s blood at any given time so that an appropriate amount of insulin can be automatically administered to help regulate the amount of glucose in the patient’s blood. This way insulin can be automatically administered depending on the patient’s specific needs such that their blood sugar levels do not go too high or too low at any particular time. This way, an equilibrium or balance can be achieved between entry of glucose into the body (e.g., the amount of glucose that is being introduced into the patient’s blood stream via meals) and how much glucose is being consumed or utilized by the patient.

While existing continuous glucose monitoring systems that employ a continuous glucose sensor work well, such continuous glucose monitoring systems are often too expensive for potential users to afford. In some cases, a patient’s insurance does not cover such therapy or the patient’s insurance may refuse to pay for the patient to use such a solution if the patient does not fall within a high enough risk category to be covered for such therapy. In addition, some patients may simply choose not to utilize continuous glucose monitoring systems for lifestyle reasons. For instance, a particular user may choose not to wear a continuous glucose monitor or sensor arrangement because it could be uncomfortable, or they choose not to wear it all the time for other reasons. This can prevent a large group of users from using a digital diabetes management solution.

Today, a large segment of the diabetes market does not utilize CGM-type therapy devices. A substantial proportion of these users include, for example, users who rely on discrete blood glucose measurements several times a day to monitor glycemia (e.g., those using blood glucose meters), or users who intermittently wear a CGM under supervision of a health care provider (HCP). Without using a CGM device on a regular basis, these approaches can make it difficult for users to manage glycemia without actively making meaningful behavioral adjustments to improve their individual management.

Accordingly, it is desirable to provide digital diabetes management solutions that are less expensive and can thus be an option for users who are unable to afford more expensive continuous glucose monitoring systems. It would be desirable to provide users with alternative solutions that can achieve the same or similar benefits of full CGM-type therapy solutions without requiring users to wear a CGM device on a regular basis and without requiring users to absorb the costs associated with wearing a CGM device on a regular basis. Furthermore, other desirable features and characteristics will become apparent from the subsequent detailed description and the appended claims, taken in conjunction with the accompanying drawings and the foregoing technical field and background.

### BRIEF SUMMARY

Methods, systems and non-transient computer-readable media are provided for optimizing sensor wear and/or longevity of a personalized model used for estimating glucose values.

In one example, a method is provided that includes selecting a time window of data for a particular user from a set of population data; applying the subset of data to a population model to generate a personalized model that is personalized for the particular user; analyzing performance of the personalized model to determine whether the personalized model satisfies performance criteria that are indicative of performance of that personalized model; repeating the steps of selecting, generating and analyzing over a number of iterations to determine a set of personalized models that satisfy each of the performance criteria; selecting, from the set of the personalized models that have been determined to satisfy each of the performance criteria, one of the personalized models that is determined to have an optimal sensor wear period as the personalized model to deployed for the particular user; and deploying the personalized model having the optimal sensor wear period as the personalized model for the particular user. Each sensor wear period can refer to a duration that a user is required to wear a sensor for in order to acquire a sufficient amount of data needed to calibrate a personalized model of that user such that it satisfies any required performance criteria. The personalized model that has the optimal sensor wear period is the personalized model that requires a minimum wear duration to acquire data needed to satisfy any required performance criteria.

Repeating can include selecting a different time window of data for the particular user during each iteration of the selecting step, and during each iteration of the applying step, applying a subset of data from the different time window of data that was selected during that iteration to the population model to generate a different personalized model that is personalized for the particular user.

The set of population data can include data for each user of a plurality of users. For each particular user, the data (from the set of population data) can include historical data that is collected over time, such as, one or more of: data from a glucose monitoring device associated with the particular user; data regarding consumption of macronutrients by the particular user; and contextual information comprising contextual activity data associated with the particular user.

The time window of data for the particular user can include a subset of data for the particular user to be used for training a population model. The time window of data is recorded over a period that corresponds to a sensor wear period. In one example, a time window that is selected during each iteration of the method is adjusted to encompass a different subset of data for the particular user to be used for training the population model.

In one example, applying the subset of data to a population model can include training the population model based on the subset of data to adapt the population model and derive the personalized model that is personalized for the particular user.

In one example, analyzing can include determining, for each of the performance criteria that are indicative of performance of that personalized model, whether the personalized model satisfies performance requirements specified by that performance criteria; and adding the personalized model to the set of the personalized models that have been determined to satisfy each of the performance criteria when the personalized model satisfies performance requirements specified by each of the performance criteria. The performance criteria used to evaluate each personalized model can include, for example, performance metrics that are required to be satisfied by the personalized model, performance thresholds that are required to be satisfied by the personalized model, or performance that are required to be satisfied by the personalized model.

In some examples, the method can include determining metrics that measure model longevity of each personalized model over time; comparing the model longevity of each personalized model to determine which personalized model has an optimal model longevity of maximum duration; selecting, from the set of the personalized models that have been determined to satisfy each of the performance criteria, one of the personalized models that is determined to have an optimized balance between the sensor wear period and model longevity as the personalized model to be deployed to the particular user. In one example, the personalized model having the optimized balance between the sensor wear period and model longevity can be selected as the personalized model for the particular user. The model longevity is a duration of time after calibration that a personalized model continues to perform within specified performance criteria when a glucose sensor is no longer available.

In some examples, the method can also include deriving the population model from the set of population data prior to selecting the time window of data. Prior to each iteration of repeating the steps of selecting, generating and analyzing, the population model can be updated to derive a new updated population model. In some examples, during each iteration of updating the population model, a new subset of the population data can be selected to derive the new updated population model, and/or one or more machine learning models can be selected that are used to derive the new updated population model by applying the new subset of the population data to the selected machine learning models.

In another example, the steps of selecting, applying, and analyzing may be repeated for each user of a plurality of users over a number of iterations to determine a set of personalized models, for each user of the plurality of users, that satisfy each of the performance criteria. A statistical analysis can be performed to determine which one of the set of personalized models that satisfy each of the performance criteria for each of the plurality of users has the optimal sensor wear period, and the one of the set of personalized models that satisfies each of the performance criteria for each of the plurality of users can be selected as the personalized model that has the optimal sensor wear period.

In accordance with some examples, the steps of selecting, applying a personalized learning process for each user, and analyzing performance to determine the optimized sensor wear period and model longevity can be repeated over a number of iterations to determine a set of personalized models, for each user of the plurality of users, that satisfy each of the performance criteria. In those implementations, an analysis can be performed across all of the personalized models (derived for each user of the plurality of users and that satisfy the performance criteria) to determine which one has the optimal sensor wear period and/or model longevity. In some implementations, the durations of the optimal sensor wear period and/or model longevity can be evaluated using any type of statistical measure, such as an average or median, to determine which one has the optimal sensor wear period and/or the optimal model longevity.

In another example, a system is provided that includes one or more hardware-based processors configured by machine-readable instructions to select a time window of data for a particular user from a set of population data; apply the subset of data to a population model to generate a personalized model that is personalized for the particular user; analyze performance of the personalized model to determine whether the personalized model satisfies performance criteria that are indicative of performance of that personalized model; repeat the steps of selecting, generating and analyzing over a number of iterations to determine a set of personalized models that satisfy each of the performance criteria; select, from the set of the personalized models that have been determined to satisfy each of the performance criteria, one of the personalized models that is determined to have an optimal sensor wear period as the personalized model to deployed for the particular user; and deploy the personalized model having the optimal sensor wear period as the personalized model for the particular user.

In another example, at least one non-transient computer-readable medium is provided. The non-transient computer-readable medium has instructions stored thereon that are configurable to cause at least one processor to perform a method that includes selecting a time window of data for a particular user from a set of population data; applying the subset of data to a population model to generate a personalized model that is personalized for the particular user; analyzing performance of the personalized model to determine whether the personalized model satisfies performance criteria that are indicative of performance of that personalized model; repeating the steps of selecting, generating and analyzing over a number of iterations to determine a set of personalized models that satisfy each of the performance criteria; selecting, from the set of the personalized models that have been determined to satisfy each of the performance criteria, one of the personalized models that is determined to have an optimal sensor wear period as the personalized model to deployed for the particular user; and deploying the personalized model having the optimal sensor wear period as the personalized model for the particular user.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter. The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a block diagram of a system for estimating glucose values for a particular user or patient in accordance with the disclosed embodiments;

FIG. 2 is a flowchart that illustrates a method for generating an estimation model for a particular patient in accordance with the disclosed embodiments;

FIG. 3 is a flowchart of a method in accordance with the disclosed embodiments;

FIG. 4 is a block diagram of a workflow process for generating a personalized model that is optimized for estimating glucose values for a particular user or patient in accordance with the disclosed embodiments;

FIG. 5 is a block diagram of a machine learning system for generating an optimized population model for estimating glucose values for a population of users or patients in accordance with the disclosed embodiments;

FIG. 6 is a block diagram of a windowed machine learning system for generating an optimized window population model for estimating glucose values for a population of users or patients in accordance with the disclosed embodiments;

FIG. 7 is a block diagram of another windowed machine learning system for generating an optimized window population model for estimating glucose values for a population of users or patients in accordance with the disclosed embodiments;

FIG. 8 is a block diagram of another windowed machine learning system for generating an optimized window population model for estimating glucose values for a population of users or patients in accordance with the disclosed embodiments;

FIG. 9 is a block diagram of another windowed machine learning system for generating an optimized window population model for estimating glucose values for a population of users or patients in accordance with the disclosed embodiments;

FIG. 10 is a block diagram of another windowed machine learning system for generating an optimized window population model for estimating glucose values for a population of users or patients in accordance with the disclosed embodiments;

FIG. 11 is a block diagram of a model explainability analysis processor for selecting an optimized population model for estimating glucose values for a population of users or patients in accordance with the disclosed embodiments;

FIG. 12 is a block diagram of a calibration optimization processor for selecting an optimized calibration model in accordance with the disclosed embodiments;

FIG. 13 is a block diagram of a calibration optimization processor for selecting optimal type(s) of inputs to be used for developing an optimized calibration model in accordance with the disclosed embodiments;

FIG. 14 is a block diagram of a machine learning system for generating an optimized personal model for estimating glucose values that are personalized for a particular user or patient in accordance with the disclosed embodiments;

FIG. 15 is a block diagram of a windowed machine learning system for generating an optimized personal model for estimating glucose values that are personalized for a particular user or patient in accordance with the disclosed embodiments;

FIG. 16 is a block diagram of another windowed machine learning system for generating an optimized personal model for estimating glucose values that are personalized for a particular user or patient in accordance with the disclosed embodiments;

FIG. 17 is a block diagram of another windowed machine learning system for generating an optimized personal model for estimating glucose values that are personalized for a particular user or patient in accordance with the disclosed embodiments;

FIG. 18 is a block diagram of another windowed machine learning system for generating an optimized personal model for estimating glucose values for a personalized a particular user or patient in accordance with the disclosed embodiments;

FIG. 19 is a block diagram that illustrates an intermittent CGM system in accordance with the disclosed embodiments;

FIG. 20 is a block diagram that illustrates another intermittent CGM system in accordance with the disclosed embodiments;

FIG. 21 is a flowchart of a method for updating an existing population model for estimating glucose values for a population of particular users to generate a new updated population model in accordance with the disclosed embodiments;

FIG. 22 is a flowchart of a method for updating an existing personalized model 2260 for estimating glucose values to generate a new updated personalized model that is personalized for a particular user in accordance with the disclosed embodiments;

FIG. 23 is a flowchart of another method for updating an existing personalized model 2260 for estimating glucose values to generate a new updated personalized model that is personalized for a particular user in accordance with the disclosed embodiments;

FIG. 24 is a flow chart of a method for optimizing a sensor wear period in accordance with the disclosed embodiments;

FIG. 25 is a flow chart of a method for optimizing sensor wear period and longevity of a personalized model used for estimating glucose values of a particular user in accordance with the disclosed embodiments; and

FIG. 26 is a flow chart of another method for optimizing sensor wear period and longevity of a personalized model used for estimating glucose values of a particular user in accordance with the disclosed embodiments.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be configurable to be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Exemplary embodiments of the subject matter described herein are implemented in conjunction with medical devices, such as portable electronic medical devices. Although many different applications are possible, the following description focuses on embodiments that incorporate an insulin infusion device (or insulin pump) as part of an infusion system deployment. For the sake of brevity, conventional techniques related to infusion system operation, insulin pump and/or infusion set operation, and other functional aspects of the systems (and the individual operating components of the systems) may not be described in detail here. Examples of infusion pumps may be of the type described in, but not limited to, U.S. Pat. Nos.: 4,562,751; 4,685,903; 5,080,653; 5,505,709; 5,097,122; 6,485,465; 6,554,798; 6,558,320; 6,558,351; 6,641,533; 6,659,980; 6,752,787; 6,817,990; 6,932,584; and 7,621,893; each of which are hereby incorporated by reference in their entirety, except for any disclaimers, disavowals, and inconsistencies, and to the extent that the incorporated material is inconsistent with the express disclosure herein, the language in this disclosure controls, and any inconsistent or conflicting information in the incorporated material is not incorporated by reference herein.

Generally, a fluid infusion device includes a motor or other actuation arrangement that is operable to linearly displace a plunger (or stopper) of a fluid reservoir provided within the fluid infusion device to deliver a dosage of fluid, such as insulin, to the body of a user. Dosage commands that govern operation of the motor may be generated in an automated manner in accordance with the delivery control scheme associated with a particular operating mode, and the dosage commands may be generated in a manner that is influenced by a current (or most recent) measurement of a physiological condition in the body of the user. For example, in a closed-loop or automatic operating mode, dosage commands may be generated based on a difference between a current (or most recent) measurement of the interstitial fluid glucose level in the body of the user and a target (or reference) glucose setpoint value. In this regard, the rate of infusion may vary as the difference between a current measurement value and the target measurement value fluctuates. For purposes of explanation, the subject matter is described herein in the context of the infused fluid being insulin for regulating a glucose level of a user (or patient); however, it should be appreciated that many other fluids may be administered through infusion, and the subject matter described herein is not necessarily limited to use with insulin.

In addition, exemplary embodiments of the subject matter described herein can be implemented in conjunction with infusion systems, infusion devices for use in such infusion systems, control electronics, control systems, patient monitoring systems, computer-based or processor-based components and devices, etc. For the sake of brevity, conventional techniques related to such infusion systems, infusion devices for use in such infusion systems, control electronics, control systems, patient monitoring systems, computer-based or processor-based components and devices, and other functional aspects of the systems (and the individual operating components of the systems) may not be described in detail here. Examples of infusion systems, infusion devices for use in such infusion systems, control electronics, control systems, patient monitoring systems, and computer-based or processor-based components and devices may be of the type described in, but not limited to, U.S. Pat. Application Serial Nos.: 16/533,470; 16/987,330; and 17/178,087; each of which are hereby incorporated by reference in their entirety, except for any disclaimers, disavowals, and inconsistencies, and to the extent that the incorporated material is inconsistent with the express disclosure herein, the language in this disclosure controls, and any inconsistent or conflicting information in the incorporated material is not incorporated by reference herein.

In one non-limiting embodiment, a method and system are provided for estimating glucose values based, for example, on data from a BG meter and an activity tracker. In other embodiments, other contextual information, such as data regarding consumption of macronutrients by the particular user (e.g., type and amount of food including consumed by the particular user) can also be used to estimate glucose values (e.g., to reconstruct continuous glucose data). Machine learning-based estimation/prediction models can be developed offline for each patient, or updated online for continuous learning, particularly during retraining periods of continuous glucose monitor (CGM) taking in current contextual data. Generic estimation/predication models can be built using patterns/instances from data collected from a similar cohort as the patient which provides a bigger data set and hence larger scope for patient characterization. Because every person is different the estimation/prediction models are uniquely trained for each patient.

For example, a customized machine learning-based estimation/prediction model can be built for each patient during a training process using CGM data. The customized machine learning-based estimation/prediction model can then be invoked at run time based on finger-stick measurements entered by the user via a blood glucose meter in addition to other contextual information such as steps, heart rate, exercise, metabolic equivalents and other parameters collected from an activity tracker, and insulin and meal information from insulin pumps (or insulin pens) or user entries, while also taking into account their medical history and demographic information so that the predictions are not only personalized based on the individual’s diabetes profile and behavior, but also equipped to account for deviations from a patient’s regular routine. The customized machine learning-based estimation/prediction model can thus provide continuous current and predictive estimates of glucose levels in real-time using finger-stick measurements entered by the user via a blood glucose meter and including any of the other contextual information mentioned above. Also, because the contextual data for a patient can change over time, the estimation/prediction models can be updated on a regular basis. In other words, because a patient’s condition can change over time, a patient can wear a continuous glucose sensor periodically (e.g., once or twice a month) to acquire data that can be used to retrain the estimation model so that it can generate more accurate estimates for that particular patient.

The functionality can reside, for example, both within a web-based application as well as a mobile application. The estimation models can be implemented/deployed at either a backend server that runs the estimation model as part of a web-based application and communicates information back to the user’s phone, insulin pump or other end device, and/or can also implemented as part of a mobile application that runs on the user’s phone or other end device and communicates information back to the insulin pump or other end device. The estimation models can also be implemented/deployed at an insulin pump. Regardless of the implementation, the applications can send push notifications to the patient to alert them in case of an estimated low or high excursion event, can receive requests from user and deliver precise, coherent answers, and can also provide a pathway for entering data which can be used to keep track of the user history as well as improve the prediction estimates.

The estimation model can be trained using a continuous glucose sensor device, and once it is trained and then personalized/tuned for a particular user/patient, the continuous glucose sensor does not need to be used on a regular basis. This can allow users/patients to rely less on a continuous glucose sensor. They only need to use a CGM periodically instead of on a regular basis.

Instead, a blood glucose meter can be used in conjunction with an activity tracker to generate inputs to the estimation model, and the estimation model can then generate estimated glucose values that approximate measured glucose values that would be measured by a continuous glucose sensor. In essence, the disclosed embodiments can allow a patient to use an activity tracker and intermittent measurements from a blood glucose meter without needing a continuous glucose sensor on a regular basis (e.g., the patient can use a continuous glucose sensor periodically for calibration, but then only use a blood glucose meter and an activity tracker in conjunction with an estimation model the remainder of the time).

In some embodiments, additional types of data can be processed by the estimation model, which can improve the accuracy of the estimates. For example, a patient can estimate their glucose values using meal data that describes what the patient has recently eaten, intermittent blood glucose measurements from a blood glucose meter (that are taken by a user, for example, via finger stick measurements), and other contextual activity data that is received on a continuous basis from an activity tracker or other source of activity information about the patient. This other contextual activity data can include data such as physical activity data (e.g., heart rate, number of steps, or metabolic equivalents, etc.), metabolic data (e.g., calories burned), etc. This information can be used via an estimation or predication module to estimate glucose values in a way that approximates, with high accuracy, blood glucose measurements that would be measured by a continuous glucose sensor without having to incur the cost of, or permanently attach, a continuous glucose sensor. When the estimated glucose values are accurate enough, they can be used, for example, to drive an insulin infusion device so that it administers insulin to a patient, or to provide notifications to a patient or health care partner to manually inject a bolus of insulin on an as needed basis depending on their estimated glucose values and other information such as activity data, meal information, etc., or alternatively to provide health care providers with information to adjust basal rate settings of an inulin infusion device so that they are appropriate for a particular patient to meet their needs. It may also be used as a medication titration advisor for individuals on other types of blood sugar lowering medications. When the estimated glucose values are not accurate enough, then this condition can be used to trigger a notification, warning, alarm or alert, for example, at another device such as at the insulin pump, the user’s smartphone or other smart device, etc. to inform the user that they should not trust the system and to recommend that the user to wear another sensor (e.g., CGM) to retrain the estimation model.

Thus, the proposed method and system can provide a new digital diabetes management solution that is cheaper and more affordable; more likely to be used by users who are unable to afford digital diabetes management systems; more likely to be approved by insurance, etc. The proposed method and system can also help users start using a digital diabetes management solution until they transition to a CGM-based system for diabetes management.

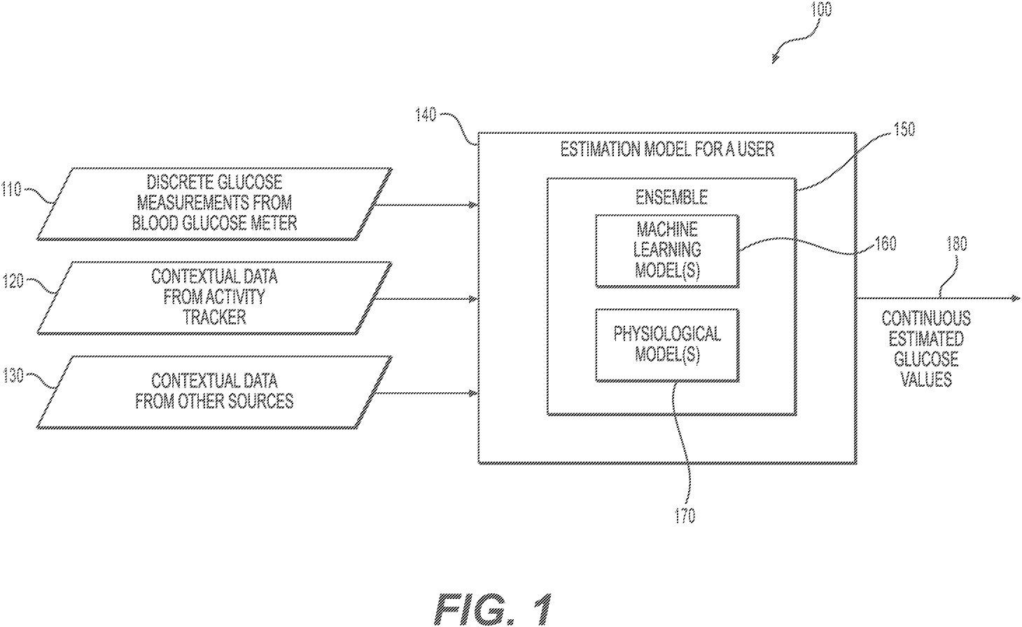

FIG. 1 is a block diagram of a system 100 for estimating glucose values for a particular user or patient in accordance with the disclosed embodiments. The system 100 includes an estimation model 140 that is configured to receive various inputs 110, 120, 130. The estimation model 140 can be executed at a processing system. The processing system can be implemented at any of the devices described herein including those illustrated and described in U.S. Pat. Application Serial Nos.: 16/533,470; 16/987,330; and 17/178,087, and more generically at a computer-based or processor-based device such as a backend server system, a client device, a medical device or other end device, such as the auxiliary sensing arrangement(s), an activity tracker device, ring, necklace, vest, etc. For instance, the processing system can be implemented at a backend server system, where the estimation model can be executed or run as part of a web application at the backend server system, and the backend server system can then communicate the estimated glucose values to a client device (e.g., patient’s smartphone), an insulin infusion device or other end device. In another embodiment, the processing system can be implemented at a client device and the estimation model can be executed as part of a mobile application at the client device. In another embodiment, the processing system can be implemented at an insulin infusion device and the estimation model can be executed as part of an application at the insulin infusion device.

In one nonlimiting embodiment, the inputs include discrete glucose measurements 110 from a blood glucose meter (BGM), contextual activity data 120 from an activity tracker or equivalent source of activity data such as a smartphone or other health related device, and optionally contextual data 130 from other sources. The contextual activity data 120 collected from the activity tracker or equivalent source of activity data (e.g., smartphone or other health related device) can include, for example, one or more of: metabolic data about the patient (e.g., calories burned); physical activity data about the user (e.g., heart rate data for the patient, exercise data for the patient, activity type, duration and intensity data, data about a number of steps taken by the patient during a time period, metabolic equivalents and other parameters); data from a fitness or exercise advisor device or application that provides fitness/exercise suggestions or recommendations to users; and other raw contextual data such as accelerometer (x,y,z) data, geolocation data, iBeacon location data, skin temperature data, ambient air temperature data, bioimpedance heart rate data, sweat data (e.g., GSR-conductance), blood pressure data, shake detection data, pedometer data, barometer data, gyroscope data, meal log data, orientation (azimuth, pitch, roll (degrees)) data; health kit data (such as data from Apple® Healthkit, Google® Fit, etc.), medication/prescription information data, user gestures data, uv light detector data, magnetometer data, respiration data, muscle activity data, pulse oximeter data, blood + interstitial fluid pH data, metabolic equivalents (METS) data, sleep quality/time data or data regarding sleep patterns of a particular user, EMR and lab data, etc. The contextual data 130 from other sources can include, for example, historical sensor glucose measurement data, historical delivery data, historical auxiliary measurement data such as historical acceleration measurement data, historical heart rate measurement data, skin conductance, and/or the like, historical event log data, historical geolocation data, and other historical or contextual data are correlated to or predictive of the occurrence of a particular event, activity, or metric for a particular patient. The estimation model 140 processes at least some of the various inputs to continuously generate estimated glucose values 180.

In one or more exemplary embodiments, the processing system utilizes machine learning to determine, based on discrete glucose measurements 110 from a blood glucose meter, contextual activity data 120 from one or more auxiliary sensing arrangement(s) (e.g., from “activity tracker” or equivalent source of activity data), and optionally contextual data 130 from other sources, to generate continuous estimated glucose values 180. Depending on the implementation, the machine learning model 160 can be a corresponding equation, function, or model for calculating the estimated glucose values 180 based on the set of input variables. Thus, the machine learning model 160 is capable of characterizing or mapping a particular combination of inputs to a value representative of the estimated glucose values 180.

Artificial intelligence is an area of computer science emphasizes the creation of intelligent machines that work and react like humans. Some of the activities computers with artificial intelligence are designed for include learning. Examples of artificial intelligence algorithms include, but are not limited to, key learning, actor critic methods, reinforce, deep deterministic policy gradient (DDPG), multi-agent deep deterministic policy gradient (MADDPG), etc. Machine learning refers to an artificial intelligence discipline geared toward the technological development of human knowledge.

Machine learning facilitates a continuous advancement of computing through exposure to new scenarios, testing and adaptation, while employing pattern and trend detection for improved decisions and subsequent, though not identical, situations. Machine learning (ML) algorithms and statistical models can be used by computer systems to effectively perform a specific task without using explicit instructions, relying on patterns and inference instead. Machine learning algorithms build a mathematical model based on sample data, known as “training data,” in order to make predictions or decisions without being explicitly programmed to perform the task. Machine learning algorithms can be used when it is infeasible to develop an algorithm of specific instructions for performing the task.

For example, supervised learning algorithms build a mathematical model of a set of data that contains both the inputs and the desired outputs. The data is known as training data and consists of a set of training examples. Each training example has one or more inputs and a desired output, also known as a supervisory signal. In the case of semi-supervised learning algorithms, some of the training examples are missing the desired output. In the mathematical model, each training example is represented by an array or vector, and the training data by a matrix. Through iterative optimization of an obj ective function, supervised learning algorithms learn a function that can be used to predict the output associated with new inputs. An optimal function will allow the algorithm to correctly determine the output for inputs that were not a part of the training data. An algorithm that improves the accuracy of its outputs or predictions over time is said to have learned to perform that task. Supervised learning algorithms include classification and regression. Classification algorithms are used when the outputs are restricted to a limited set of values, and regression algorithms are used when the outputs may have any numerical value within a range. Similarity learning is an area of supervised machine learning closely related to regression and classification, but the goal is to learn from examples using a similarity function that measures how similar or related two objects are.

Reinforcement learning is an area of machine learning concerned with how software agents ought to take actions in an environment so as to maximize some notion of cumulative reward. Due to its generality, the field is studied in many other disciplines, such as game theory, control theory, operations research, information theory, simulation-based optimization, multi-agent systems, swarm intelligence, statistics, and genetic algorithms. In machine learning, the environment is typically represented as a Markov Decision Process (MDP). Many reinforcement learning algorithms use dynamic programming techniques. Reinforcement learning algorithms do not assume knowledge of an exact mathematical model of the MDP and are used when exact models are infeasible.

In accordance with one non-limiting implementation, the estimation model 140 can be an ensemble model 150. An ensemble model 150 includes two or more related, but different analytical models that are run or executed at the same time, where the results generated by each can then be synthesized into a single score or spread in order to improve the accuracy of predictive analytics. To explain further, in predictive modeling and other types of data analytics, a single model based on one data sample can have biases, high variability or outright inaccuracies that can affect the reliability of its analytical findings. By combining different models or analyzing multiple samples, the effects of those limitations can be reduced to provide better information. As such, ensemble methods can use multiple machine learning algorithms to obtain better predictive performance than could be obtained from any of the constituent learning algorithms alone.

An ensemble is a supervised learning algorithm because it can be trained and then used to make predictions. The trained ensemble, therefore, represents a single hypothesis that is not necessarily contained within the hypothesis space of the models from which it is built. Thus, ensembles can be shown to have more flexibility in the functions they can represent. An ensemble model can include a set of individually trained classifiers (such as neural networks or decision trees) whose predictions are combined.

For instance, one common example of ensemble modeling is a random forest model which is a type of analytical model that leverages multiple decision trees and is designed to predict outcomes based on different variables and rules. A random forest model blends decision trees that may analyze different sample data, evaluate different factors or weight common variables differently. The results of the various decision trees are then either converted into a simple average or aggregated through further weighting. The emergence of Hadoop and other big data technologies has allowed greater volumes of data to be stored and analyzed, which can allow analytical models to be run on different data samples.

In some embodiments, the ensemble model 150 can include or be built using at least one machine learning model 160 and a physiological model 170. The physiological model 170 can be, for example, a population-based model of one or more equations (e.g., a set of differential equations) that are generally applicable to any patient, but has different variables or parameters that are weighted in accordance with a particular patient’s individual parameters to mimic the physiology of that patient. In other words, because each patient’s physiological response may vary from the rest of the population, the relative weightings applied to the respective variables of the physiological model 170 may also vary from other patients. The physiological model 170 can be used in conjunction with the machine learning model(s) 160 to help confine estimations or predictions within a realistic range.

Depending on the implementation, any number of machine learning models can be combined to optimize the ensemble model 150. Examples of machine learning algorithms or models that can be implemented at the machine learning model 160 can include, but are not limited to: regression models such as linear regression, logistic regression, and K-means clustering; one or more decision tree models (e.g., a random forest model); one or more support vector machines; one or more artificial neural networks; one or more deep learning networks (e.g., at least one recurrent neural network, sequence to sequence mapping using deep learning, sequence encoding using deep learning, etc.); fuzzy logic based models; genetic programming models; Bayesian networks or other Bayesian techniques, probabilistic machine learning models; Gaussian processing models; Hidden Markov models; time series methods such as Autoregressive Moving Average (ARMA) models, Autoregressive Integrated Moving Average (ARIMA) models, Autoregressive conditional heteroskedasticity (ARCH) models; generalized autoregressive conditional heteroskedasticity (GARCH) models; moving-average (MA) models or other models; and heuristically derived combinations of any of the above, etc. The types of machine learning algorithms differ in their approach, the type of data they input and output, and the type of task or problem that they are intended to solve.

A Hidden Markov model (HMM) is a statistical Markov model in which the system being modeled is assumed to be a Markov process with unobserved (hidden) states. An HMM can be considered as the simplest dynamic Bayesian network. A Bayesian network, belief network or directed acyclic graphical model is a probabilistic graphical model that represents a set of random variables and their conditional independence with a directed acyclic graph (DAG). Bayesian networks that model sequences of variables are called dynamic Bayesian networks. Generalizations of Bayesian networks that can represent and solve decision problems under uncertainty are called influence diagrams.

Support vector machines (SVMs), also known as support vector networks, are a set of related supervised learning methods used for classification and regression. Given a set of training examples, each marked as belonging to one of two categories, an SVM training algorithm builds a model that predicts whether a new example falls into one category or the other. An SVM training algorithm is a non-probabilistic, binary, linear classifier. In addition to performing linear classification, SVMs can efficiently perform a non-linear classification using what is called the kernel trick, implicitly mapping their inputs into high-dimensional feature spaces.

Decision tree learning uses a decision tree as a predictive model to go from observations about an item (represented in the branches) to conclusions about the item’s target value (represented in the leaves). Tree models where the target variable can take a discrete set of values are called classification trees; in these tree structures, leaves represent class labels and branches represent conjunctions of features that lead to those class labels. Decision trees where the target variable can take continuous values (typically real numbers) are called regression trees. In decision analysis, a decision tree can be used to visually and explicitly represent decisions and decision making.

Deep learning algorithms can refer to a collection of algorithms used in machine learning, that are used to model high-level abstractions and data through the use of model architectures, which are composed of multiple nonlinear transformations. Deep learning is a specific approach used for building and training neural networks. Deep learning consists of multiple hidden layers in an artificial neural network. Examples of deep learning algorithms can include, for example, Siamese networks, transfer learning, recurrent neural networks (RNNs), long short term memory (LSTM) networks, convolutional neural networks (CNNs), transformers, etc. For instance, deep learning approaches can make use of autoregressive Recurrent Neural Networks (RNN), such as the long short-term memory (LSTM) and the Gated Recurrent Unit (GRU). One neural network architecture for time series forecasting using RNNs (and variants) is an autoregressive seq2seq neural network architecture, which acts as an autoencoder.

In some embodiments, the ensemble model 150 can include one or more deep learning algorithms. It should be noted that any number of different machine learning techniques may also be utilized. Depending on the implementation, the ensemble model 150 can be implemented as a bootstrap aggregating ensemble algorithm (also referred to as a bagging classifier method), as a boosting ensemble algorithm or classifier algorithm, as a stacking ensemble algorithm or classifier algorithm, as bucket of models, ensemble algorithms, as Bayes optimal classifier algorithms, as Bayesian parameter averaging algorithms, as Bayesian model combination algorithms, etc.

Bootstrap aggregating, often abbreviated as bagging, involves having each model in the ensemble vote with equal weight. In order to promote model variance, bagging trains each model in the ensemble using a randomly drawn subset of the training set. As an example, the random forest algorithm combines random decision trees with bagging to achieve very high classification accuracy. A bagging classifier or ensemble method creates individuals for its ensemble by training each classifier on a random redistribution of the training set. Each classifier’s training set can be generated by randomly drawing, with replacement, N examples - where N is the size of the original training set; many of the original examples may be repeated in the resulting training set while others may be left out. Each individual classifier in the ensemble is generated with a different random sampling of the training set. Bagging is effective on “unstable” learning algorithms (e.g., neural networks and decision trees), where small changes in the training set result in large changes in predictions.

By contrast, boosting involves incrementally building an ensemble by training each new model instance to emphasize the training instances that previous models mis-classified. In some cases, boosting has been shown to yield better accuracy than bagging, but it also tends to be more likely to over-fit the training data. A boosting classifier can refer to a family of methods that can be used to produce a series of classifiers. The training set used for each member of the series is chosen based on the performance of the earlier classifier(s) in the series. In boosting, examples that are incorrectly predicted by previous classifiers in the series are chosen more often than examples that were correctly predicted. Thus, boosting attempts to produce new classifiers that are better able to predict examples for which the current ensemble’s performance is poor. A common implementation of boosting is Adaboost, although some newer algorithms are reported to achieve better results.

Stacking (sometimes called stacked generalization) involves training a learning algorithm to combine the predictions of several other learning algorithms. Stacking works in two phases: multiple base classifiers are used to predict the class, and then a new learner is used to combine their predictions with the aim of reducing the generalization error. First, all of the other algorithms are trained using the available data, then a combiner algorithm is trained to make a final prediction using all the predictions of the other algorithms as additional inputs. If an arbitrary combiner algorithm is used, then stacking can theoretically represent any of the ensemble techniques described in this article, although, in practice, a logistic regression model is often used as the combiner.

A “bucket of models” is an ensemble technique in which a model selection algorithm is used to choose the best model for each problem. When tested with only one problem, a bucket of models can produce no better results than the best model in the set, but when evaluated across many problems, it will typically produce much better results, on average, than any model in the set. One common approach used for model-selection is cross-validation selection (sometimes called a “bake-off contest”). Cross-validation selection can be summed up as try them all with the training set and pick the one that works best. Gating is a generalization of Cross-Validation Selection. It involves training another learning model to decide which of the models in the bucket is best-suited to solve the problem. Often, a perceptron is used for the gating model. It can be used to pick the “best” model, or it can be used to give a linear weight to the predictions from each model in the bucket. When a bucket of models is used with a large set of problems, it may be desirable to avoid training some of the models that take a long time to train. Landmark learning is a meta-learning approach that seeks to solve this problem. It involves training only the fast (but imprecise) algorithms in the bucket, and then using the performance of these algorithms to help determine which slow (but accurate) algorithm is most likely to do best.

The Bayes optimal classifier is a classification technique. It is an ensemble of all the hypotheses in the hypothesis space. On average, no other ensemble can outperform it. The naive Bayes optimal classifier is a version of this that assumes that the data is conditionally independent on the class and makes the computation more feasible. Each hypothesis is given a vote proportional to the likelihood that the training dataset would be sampled from a system if that hypothesis were true. To facilitate training data of finite size, the vote of each hypothesis is also multiplied by the prior probability of that hypothesis. The hypothesis represented by the Bayes optimal classifier, however, is the optimal hypothesis in ensemble space (the space of all possible ensembles.

Bayesian parameter averaging (BPA) is an ensemble technique that seeks to approximate the Bayes optimal classifier by sampling hypotheses from the hypothesis space and combining them using Bayes’ law. Unlike the Bayes optimal classifier, Bayesian model averaging (BMA) can be practically implemented. Hypotheses are typically sampled using a Monte Carlo sampling technique such as MCMC. For example, Gibbs sampling may be used to draw hypotheses that are representative of a distribution. It has been shown that under certain circumstances, when hypotheses are drawn in this manner and averaged according to Bayes’ law, this technique has an expected error that is bounded to be at most twice the expected error of the Bayes optimal classifier.

Bayesian model combination (BMC) is an algorithmic correction to Bayesian model averaging (BMA). Instead of sampling each model in the ensemble individually, it samples from the space of possible ensembles (with model weightings drawn randomly from a Dirichlet distribution having uniform parameters). This modification overcomes the tendency of BMA to converge toward giving all of the weight to a single model. Although BMC is somewhat more computationally expensive than BMA, it tends to yield dramatically better results. The results from BMC have been shown to be better on average (with statistical significance) than BMA, and bagging. The use of Bayes’ law to compute model weights necessitates computing the probability of the data given each model. Typically, none of the models in the ensemble are exactly the distribution from which the training data were generated, so all of them correctly receive a value close to zero for this term. This would work well if the ensemble were big enough to sample the entire model-space, but such is rarely possible. Consequently, each pattern in the training data will cause the ensemble weight to shift toward the model in the ensemble that is closest to the distribution of the training data. It essentially reduces to an unnecessarily complex method for doing model selection. The possible weightings for an ensemble can be visualized as lying on a simplex. At each vertex of the simplex, all of the weight is given to a single model in the ensemble. BMA converges toward the vertex that is closest to the distribution of the training data. By contrast, BMC converges toward the point where this distribution projects onto the simplex. In other words, instead of selecting the one model that is closest to the generating distribution, it seeks the combination of models that is closest to the generating distribution. The results from BMA can often be approximated by using cross-validation to select the best model from a bucket of models. Likewise, the results from BMC may be approximated by using cross-validation to select the best ensemble combination from a random sampling of possible weightings.

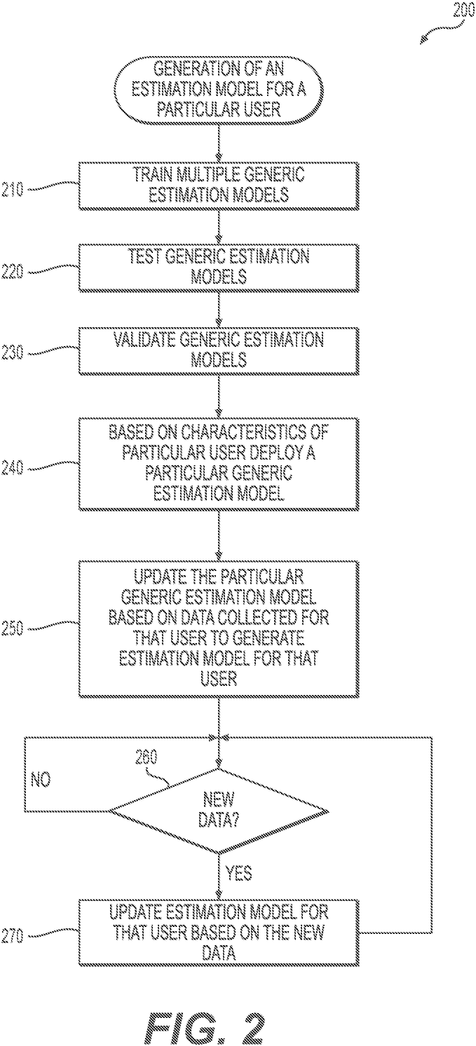

FIG. 2 is a flowchart that illustrates a method 200 for generating an estimation model for a particular patient in accordance with the disclosed embodiments. The method 200 begins at 210 where a computing system trains multiple generic estimation models. Each generic estimation model can be modeled for a particular group of users. At 220, the computing system tests the generic estimation models, and then at 230, the computing system validates the generic estimation models. For example, in some embodiments, the computing system can train multiple population-base models for type 1 and type 2 patients using different combinations of demographic parameters such as age, gender, location and diabetes history such as diabetes onset, years on insulin, therapy type using existing BG, insulin, meal information, activity data from an activity tracker, and CGM data to train, test and validate the models.

Once all the generic estimation models have been validated, the method 200 proceeds to 240, where the computing system determines, based on characteristics of a particular user or patient, an applicable generic estimation model that is the best fit for that particular patient based on characteristics of that particular patient. The particular generic estimation model that is applicable for a particular patient varies based on the characteristics of that particular patient.

At 250, the computing system can then update the particular generic estimation model that was deployed at 240 based on data that was collected for that particular patient to generate an estimation model for that particular patient. In other words, parameters of the generic estimation model that was selected and deployed 240 can be updated and fine-tuned at 250 based on data that was collected for the particular patient in order to customize that generic estimation model such that it becomes an estimation model for that particular patient.

After the estimation model for that particular patient has been generated at 250 it can be executed and updated any time new data is received for that patient. For example, in some embodiments, the new data can come from the blood glucose meter data (e.g., new blood glucose measurements), new contextual activity data that is received for that patient from the activity tracker, and/or new contextual data that is received for that patient from other sources. In one implementation, the estimation model for that particular patient can be updated each time there is a new data from the BGM. In another embodiment, the new data can be from a continuous glucose monitor. For instance, the CGM can be used periodically to collect CGM data for the particular patient for a certain period of time (e.g., for two weeks), and this newly collected CGM data can then be used to assess performance of the estimation model and/or update or re-train the estimation model for that particular patient. In addition, the CGM data can also be used to train and update the generic estimation models that were described above with reference to step 210. Furthermore, in some implementations, the CGM data can also be used at 240 to select and deploy a different one of the generic estimation models for the particular patient.

As such, at 260, the computing system regularly determines whether new data is received, and if so, the method proceeds to 270 where the estimation model for that particular patient is updated based on the new data to generate an updated estimation model that can be executed at a processing system (as will be described below).

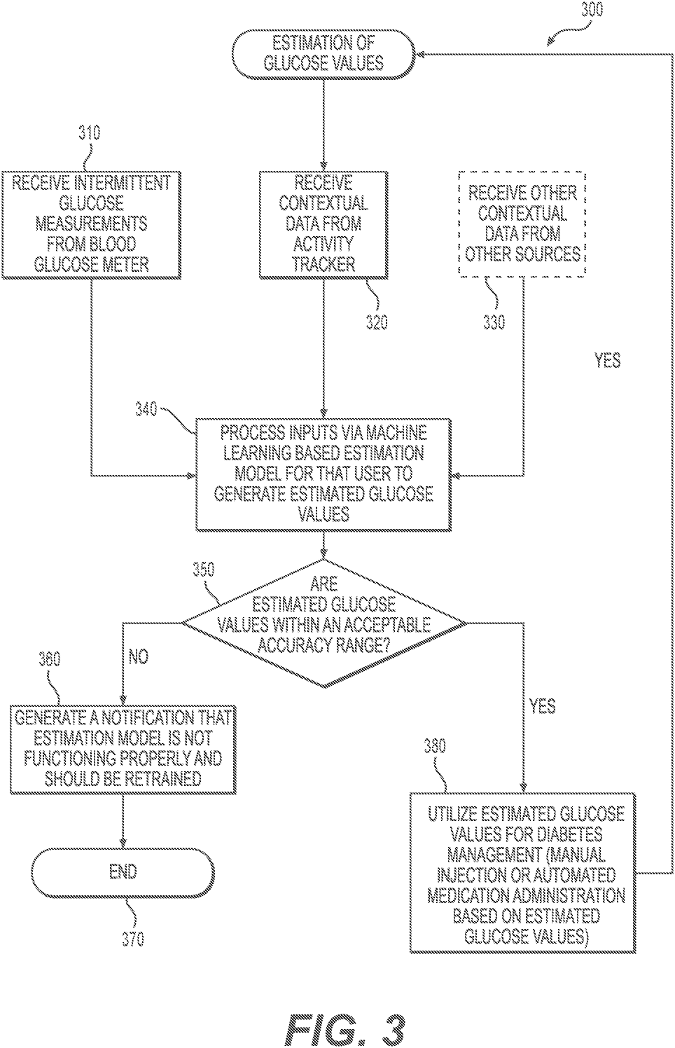

After the estimation model for that particular patient is updated based on the new data at 270, the method loops to 260. As the estimation model for the particular patient is updated it becomes more fine-tuned and can be used to generate better estimated glucose values for the patient. In any point the estimation model can be used to estimate glucose values for the patient. One non-limiting embodiment will now be described with respect to FIG. 3 where an estimation model is used to estimate glucose values for a patient.

FIG. 3 is a flowchart of a method 300 in accordance with the disclosed embodiments. At 310, a processing system can receive intermittent glucose measurements from a blood glucose meter. At 320 the processing system can also receive contextual activity data from an activity tracker. At 330, the processing system can also optionally receive other contextual data from other sources that are described above. In some embodiments, a first set of inputs comprising a sequence of intermittent blood glucose measurements are received from the calibrated blood glucose meter, and a second set of user inputs comprising contextual activity data collected from the user by an activity tracker are received from the activity tracker. Optionally, the other contextual data from other sources can include, for example, a third set of inputs from an insulin infusion device comprising insulin dispensed by the insulin infusion device and/or a fourth set of inputs comprising user entries that describe meal information.

At 340, a processing system can execute the machine learning-based estimation model for that patient to process the various inputs from steps 310, 320, 330 to generate a sequence or set of estimated glucose values for that particular patient. The estimated glucose values are analogous to glucose measurements that can be taken by a glucose sensor in that the estimated glucose values estimate continuous glucose sensor measurement values. However, the processing system can generate the set of estimated glucose values without using information from a continuous glucose sensor arrangement. This can eliminate the need for a continuous glucose sensor, which can be a relatively expensive component with respect to a blood glucose meter, and thus saves patients a significant cost. It may also support intermittent CGM users that take “sensor vacations” due to various reasons which may include access, discomfort, seasonal needs, etc. In some embodiments, only the first set of inputs and the second set of user inputs are processed via the estimation model to generate the set of estimated glucose values. As used herein, “estimated glucose value,” and “estimated glucose concentration,” and the like may refer to a glucose level that has been estimated via the machine learning-based estimation model. In some embodiments, the estimated glucose values estimate and track interstitial or sensor glucose values (e.g., that are generated by a sensing arrangement such as a CGM device that has been calibrated using measured blood glucose values measured by a blood glucose meter).

Steps 350-380 are optional and relate to when and how the estimated glucose values can be used within the context of an insulin management system for diabetes management. For example, in one implementation, at 350, the computing system can monitor the set or sequence of estimated glucose values (e.g., discrete estimates of blood glucose levels) to determine if the sequence of the estimated glucose values are within an acceptable accuracy range that allows them to be used by an insulin management system. How the computing system can determine if the sequence of the estimated glucose values are within an acceptable accuracy range can vary depending on the implementation. For example, in some embodiments, the computing system can determine if the sequence of the estimated glucose values are within an acceptable accuracy range by measuring a difference between estimated glucose value and intermittent glucose measurements from a blood glucose meter (or equivalent device) and then comparing that difference to an accuracy threshold. In another embodiment, the computing system can determine if the sequence of the estimated glucose values are within an acceptable accuracy range by measuring difference between estimated glucose value and periodic measurements that were taken by a CGM device and then comparing that difference to an accuracy threshold.

When the estimated glucose values are determined (at 350) to not be within an acceptable accuracy range that allows them to be used by an insulin management system, the method 300 proceeds to 360, where a notification or an alert can be generated that can be perceptible to the patient or another user who is monitoring the patient. For example, in some embodiments, when the estimated glucose values are not in acceptable accuracy ranges, a notification or alert can be generated to inform the user that they should not trust the estimated glucose values being generated by the estimation model and to recommend that the user to wear another sensor (e.g., CGM) to retrain the estimation model. Any number of alerting methodologies can be employed at step 360, including, but not limited to, presenting (via a display device) graphical elements and text associated with notifications and alerts for the glucose control system (see reference 100, FIG. 1). In some embodiments, notifications and alerts can be presented by an insulin pump device associated with a computing device and/or presented via a communicatively coupled personal computing device (e.g., a laptop computer, a tablet computer, a smartphone). Such notifications and alerts may include, without limitation: audio alerts (e.g., sound effects, articulated speech alerts, alarms), visual alerts (e.g., graphical elements and text presented via user interface or display of the insulin delivery pump, text effects, flashing or otherwise activated lights, color changes), text alerts (e.g., a text-based message transmitted via Short Message Service (SMS), an email message transmitted via the internet), and/or any other type of alert generated to attract the attention of the user. In addition, in some embodiments, when it is determined (at 350) that the estimated glucose values are not within an acceptable accuracy range (that allows them to be used by an insulin management system), one or more displays may be controlled so that the estimated glucose values are not presented to the user. Following 360, the method 300 can then end at 370.

When the estimated glucose values are determined (at 350) to be within an acceptable accuracy range that allows them to be used by an insulin management system, the method 300 proceeds to 380, where the estimated glucose values can be utilized the insulin delivery system, and estimates can continue to be generated in near real-time. The actions that can be taken at the insulin delivery system based on the estimated glucose values vary depending on the implementation. For example, actions that can be taken at the insulin delivery system based on the estimated glucose values can include controlling an insulin infusion device based on the estimated glucose values so that it administers insulin to a patient, or generating a notification or an alert, based on the estimated glucose values, that can be perceptible to the patient or another user who is monitoring the patient. As will be described below, the corrective actions and the notifications or alerts that can be generated vary depending on the implementation.

For example, in one implementation of step 380, the computing system operates cooperatively with an insulin delivery system and the machine learning-based estimation model to monitor estimated glucose levels of a patient, and to identify abnormalities in the estimated glucose levels. For example, the computing system can monitor the set or sequence of estimated glucose values (e.g., discrete estimates of blood glucose levels) to determine if the sequence of the estimated glucose values are within a low range, a high range, or an acceptable range. For instance, hypoglycemia is a deficiency of glucose in the bloodstream of the patient or user of the insulin delivery pump. Hypoglycemia may be generally associated with glucose values below 70 milligrams per deciliter (mg/dL), depending on certain patient-specific factors. Hyperglycemia is an excess of glucose in the bloodstream of the patient or user of the insulin delivery pump and may be generally associated with glucose values above 180 mg/dL, depending on certain patient-specific factors. Normal, healthy blood glucose levels, over every 24-hour cycle, generally range between 70 and 160 mg/dL. An abnormality in a sequence of blood glucose levels may include blood glucose levels below a predefined hypoglycemia threshold, blood glucose levels above a predefined hyperglycemia threshold, a decreasing blood glucose trend approaching or below a predefined hypoglycemia threshold, an increasing blood glucose trend approaching or above a predefined hyperglycemia threshold, or the like.

The abnormality indicates that a corrective action is required to maintain blood glucose stability at appropriate levels. Abnormalities in blood glucose levels require intervention to bring the patient’s blood glucose levels into a safe and healthy range. As such, when the estimated glucose values are not within an acceptable range, the method 300 can proceed to 380, where a corrective action can be taken at the insulin infusion device to deliver the patient an amount of insulin required to adequately treat the blood glucose abnormality to bring the blood glucose levels of the patient within normal limits and/or regulate their blood sugar levels. For example, in some embodiments, the estimated glucose values can be used to control and drive an insulin infusion device so that it administers insulin to a patient.

Exemplary embodiments of corrective actions are disclosed in, for example, in U.S. Pat. Application Serial No. 16/129,552, filed Sep. 12, 2018, entitled “SYSTEMS AND METHODS FOR ALERTING A USER TO DETECTED EFFECTS OF A RECENT THERAPY ADJUSTMENT,” and assigned to the assignee of the present invention, which is incorporated herein by reference in its entirety. Any number of corrective actions or intervention methodologies can be employed at step 380, such as, administration of a bolus of insulin (to decrease blood glucose levels) or administration of a quantity of carbohydrates, glucose, or other medication (to increase blood glucose levels). In other words, the corrective action may include the administration of a bolus of insulin (to decrease hyperglycemic blood glucose levels) or the administration of a dose of carbohydrates (to increase hypoglycemic blood glucose levels).

In addition, in some embodiments, at step 380, the computing system operates cooperatively with an insulin delivery system (e.g., insulin pump or manual insulin injection device such as an insulin pen) and the machine learning-based estimation model to monitor estimated glucose levels of a patient, and to generate a notification or an alert, based on the estimated glucose values, that can be perceptible to the patient or another user who is monitoring the patient (e.g., to inform the patient or the user of any abnormalities that are detected when the estimated glucose values are within the low range that is too low or within the high range that is too high). For instance, a notification or alert can be sent to the user to alert them that estimated glucose values are abnormal and help drive decision support for medication administration. Exemplary embodiments of alerts are disclosed, for example, in U.S. Pat. Application Serial No. 16/129,552, filed Sep. 12, 2018, entitled “SYSTEMS AND METHODS FOR ALERTING A USER TO DETECTED EFFECTS OF A RECENT THERAPY ADJUSTMENT,” and assigned to the assignee of the present invention, which is incorporated herein by reference in its entirety. Any number of alerting methodologies can be employed at step 380, including, but not limited to, presenting (via a display device) graphical elements and text associated with notifications and alerts for the glucose control system. In some embodiments, notifications and alerts can be presented by an insulin pump device associated with a computing device and/or presented via a communicatively coupled personal computing device (e.g., a laptop computer, a tablet computer, a smartphone). Such notifications and alerts may include, without limitation: audio alerts (e.g., sound effects, articulated speech alerts, alarms), visual alerts (e.g., graphical elements and text presented via user interface or display of the insulin delivery pump, text effects, flashing or otherwise activated lights, color changes), text alerts (e.g., a text-based message transmitted via Short Message Service (SMS), an email message transmitted via the internet), and/or any other type of alert generated to attract the attention of the user.

For example, a presentation module can identify appropriate graphical elements for presentation via a display device that identify a current estimated blood glucose level (e.g., various icons, text, and/or graphical elements associated with estimated blood glucose levels of a patient), a detected indication of a change of direction in the sequence of blood glucose levels, timing data associated with a detected indication of a change of direction in the sequence of blood glucose levels, an indication that a corrective action should be taken by a user, confirmation of a user-input indication that a corrective action has been completed by a user, and the like.

For example, a display device can display, render, or otherwise convey one or more graphical representations or images associated with blood glucose levels, an indication of a change of direction in a blood glucose trend, and/or notifications or alerts associated with blood glucose levels on the display device. In some embodiments, an alert can notify the patient of an over-corrected or under-corrected abnormality. For instance, in one implementation, an alert can be presented via the insulin delivery pump or a manual insulin injection device (e.g., insulin pen), such that the patient is informed that an administered corrective action has not affected the blood glucose levels of the patient in the desired manner (e.g., that the corrective action has over-corrected or under-corrected the abnormality).

In some embodiments, an alert can be presented to prompt a user to perform a corrective action, in response to a detected abnormality (e.g., blood glucose levels that are too high or too low). For example, when the abnormality includes blood glucose levels that are too high, the user (patient or health care partner) can be prompted to input a command for the insulin delivery pump to administer a bolus of insulin, via the insulin deliver pump, to treat the high blood glucose levels. Alternatively, a notification can provide health care providers with information to adjust basal rate settings of an inulin infusion device so that they are appropriate for a particular patient to meet their needs. In another embodiment, when the abnormality includes blood glucose levels that are too high, the user (patient or health care partner) can be prompted to input a command at a manual insulin injection device (e.g., insulin pen) to administer insulin. By contrast, when the abnormality includes blood glucose levels that are too low, the alert can prompt the user to administer a dose of carbohydrates to treat the low blood glucose levels.

Additionally, the estimated glucose values can be utilized by other downstream systems, such as a fitness or exercise advisor device or application that provides fitness/exercise suggestions or recommendations to users. For instance, the estimated glucose values can be utilized in conjunction with a predicted exercise level in the short future (using similar exercise pattern from the past few minutes) to tell patient if keeping current level of activity will reduce hyperglycemia or introduce hypoglycemia.

After 380, the method 380 can loop to the start, and then proceeds to steps 310, 320 and 330 to wait and receive new data regarding blood glucose measurements from a blood glucose meter, contextual activity data from an activity tracker and/or other contextual data from other sources and the model would be retrained.

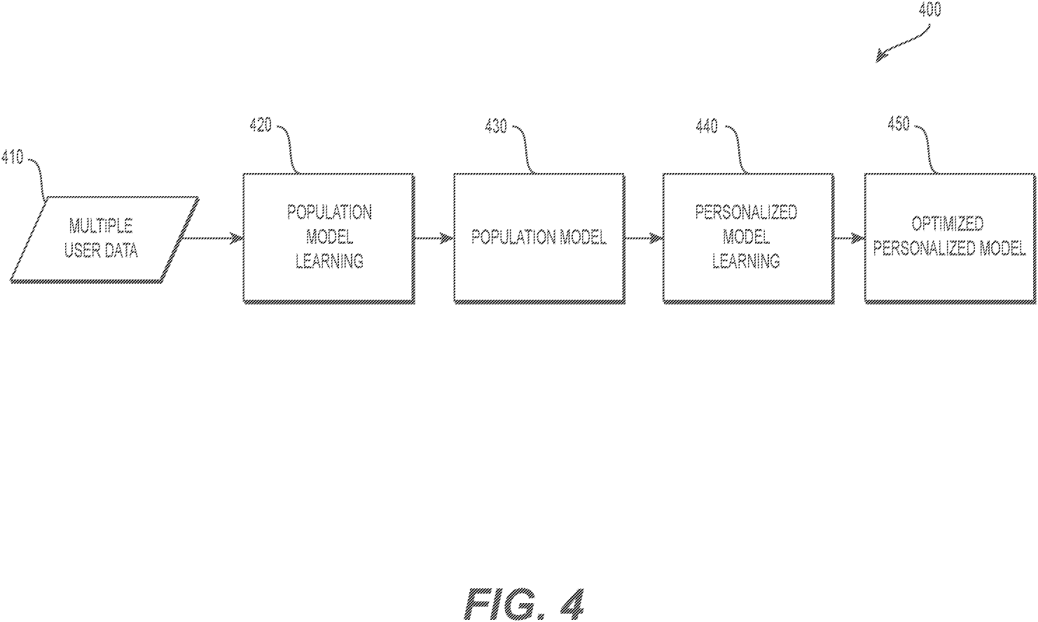

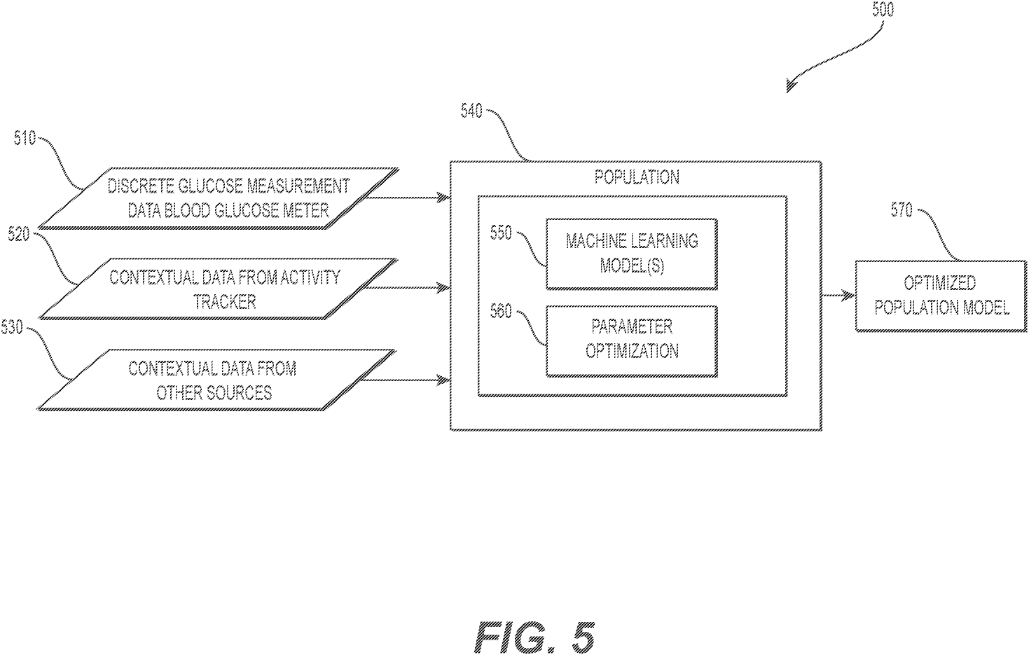

FIG. 4 is a block diagram of a workflow process 400 for generating a personalized model 450 that is optimized for estimating glucose values for a particular user or patient in accordance with the disclosed embodiments. The workflow process 400 starts at 410 where data for multiple users is collected. Examples of this data will be described in greater detail below. At 420, population model learning processes can be performed to generate a set of one or more population models. In one non-limiting embodiment, a deep learning network can be used to develop a generic estimation model that is based on learned characteristics of a population of users. Nonlimiting examples of the various processes that can be performed as part of 420 will be described below with reference to FIGS. 5 through 13.

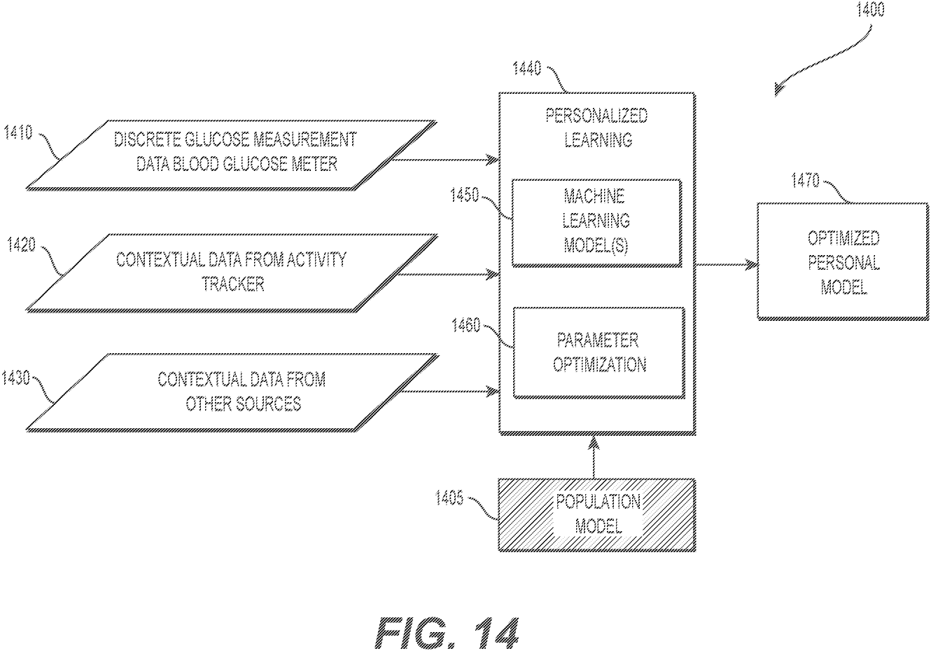

At 430, at least one of the population models can be selected, and at 440, personalized model learning processes can be performed to personalize a selected population model for each particular user. In one non-limiting embodiment, once the population model is developed, the generic, estimation model can be adapted or trained, based on learned characteristics of a particular user (e.g., patient), to generate a personalized estimation model that is optimized for estimating blood glucose of that particular user. The personalized model learning processes 440 can be performed for any number of users to personalize a population model 430 selected for each user to generate a personalized model 450 that is personalized for each user. Nonlimiting examples of the various processes that can be performed as part of 430 will be described below with reference to FIGS. 14 through 20.

Once the personalized model is generated it can be used for a variety of purposes including to provide insulin therapy to a particular user without the need for a glucose sensor such as a continuous glucose monitor.

Various embodiments of population model learning processes that can be performed will now be described below with reference to FIGS. 5 through 13. In those descriptions, like reference numbers refer to similar elements throughout the figures.

FIG. 5 is a block diagram of a machine learning system 500 for generating an optimized population model 570 for estimating glucose values for a population of users or patients in accordance with the disclosed embodiments.

During this part of the workflow process data for multiple users is collected for processing. In this example, the data that is collected can include, but is not limited to, discrete glucose measurement data 510 from the blood glucose meter or other sensor arrangement that provides discrete glucose measurements, contextual data 520 from the source of user activity data (e.g., from an activity tracker, electrodermal activity sensor, temperature sensor, oxygen monitor, etc.), and contextual data 530 from the other sources such as nutritional information about meals consumed by a user, insulin delivered to the user by an insulin infusion device of the user.

Data for each of the inputs can be collected for any number of users for a time period. Stated differently, for each user within a population, data can be collected for different input channels. Each of the different input channels can represent a different variable being measured for each user. In some embodiments, the different input channels can include (1) time of day, (2) a blood glucose level, (3) a blood glucose (BG) measurement from a sensor such as a blood glucose meter, activity data including (4) heart rate (beats per minute), (5) METs and (6) number of steps, (7) active insulin, (8) carbohydrates on board, etc. Active insulin can refer to bolus insulin that has already been delivered to a user’s body, but that has not yet been used. An active insulin setting can be considered in determining any active insulin still in a user’s body from their prior boluses and that could continue to lower their blood glucose.

In some embodiments, the discrete glucose measurement data 510, contextual data 520 from the source of user activity data and contextual data 530 from the other sources can then be processed at a population learning processor 540 to generate the optimized population model 570. In this example, as part of the learning process, the population learning processor 540 can perform various processing tasks with respect to the discrete glucose measurement data 510 from the blood glucose meter, contextual data 520 from the source of user activity data and contextual data 530 from the other sources to generate the optimized population model 570. For example, the population learning processor 540 can apply various machine learning models 550 including any described herein, such as deep learning models including but not limited to CNNs and RNNs, to the discrete glucose measurement data 510 from the blood glucose meter, contextual data 520 from the source of user activity data and contextual data 530 from the other sources. For instance, in some embodiments, a deep learning network can perform a deep learning process to process a feature matrix to generate a sequence of estimated blood glucose values.

The population learning processor 540 can also perform various parameter optimizations based on the discrete glucose measurement data 510 from the blood glucose meter, contextual data 520 from the source of user activity data and contextual data 530 from the other sources. For example, one or more machine learning or deep learning model(s) can learn the transfer function or mapping from a series of inputs (e.g., discrete glucose measurement data 510 from the blood glucose meter, contextual data 520 from the source of user activity data and contextual data 530 from the other sources) to a glucose measurement from a glucose sensor. This mapping can be on any time interval such that the model estimates anywhere from a single glucose value to a series of glucose values continuous in time. Each machine learning or deep learning model may have one or more parameters that need to be identified to specify the mathematical transfer function. Parameter optimization generally includes an objective function that is minimized. The objective function measures the mathematical agreement between the estimated output of the model with the actual measured data. Parameters are typically interactively adjusted until the objective function is optimized. The optimization process terminates once the level of agreement reaches a desired threshold or no longer improves.

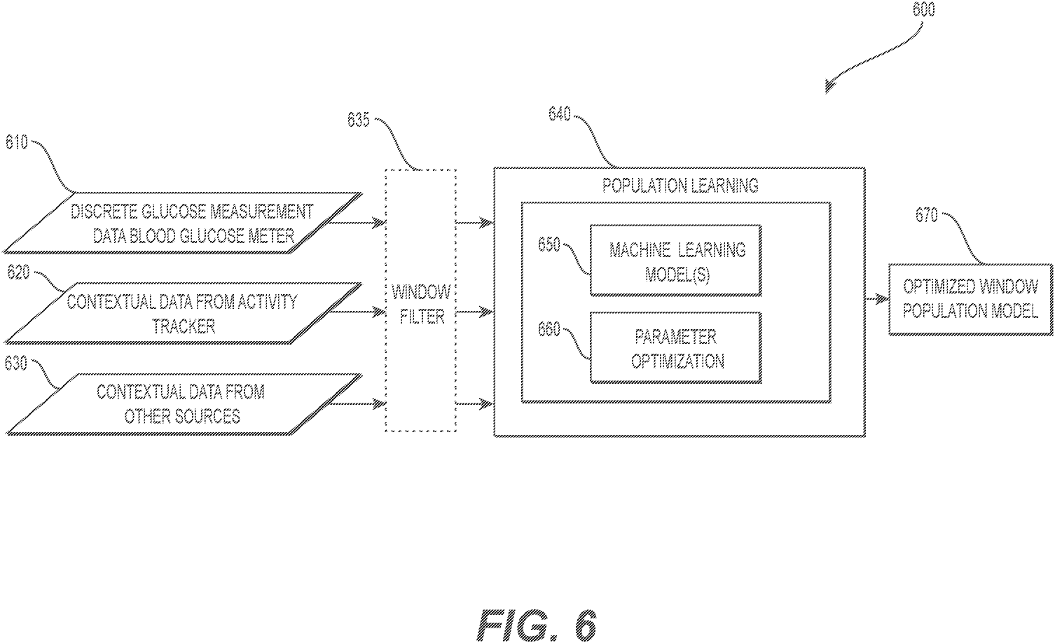

FIG. 6 is a block diagram of a windowed machine learning system 600 for generating an optimized window population model 670 for estimating glucose values for a population of users or patients in accordance with the disclosed embodiments. The structure of the windowed machine learning system 600 is similar to the structure of the machine learning system 500 described above with reference to FIG. 5. As such, components or blocks shown in FIG. 5 will not be described in detail again in conjunction with FIG. 6.

As in FIG. 5, data for multiple users is collected for processing that can include, but is not limited to, discrete glucose measurement data 610 from the blood glucose meter, contextual data 620 from the source of user activity data, and contextual data 630 from the other sources. However, in this embodiment, prior to providing the data to the population learning processor 640 for processing, the data is provided to a window filter 635. The window filter 635 can split the data into different time windows. In other words, the collected data can be sequentially split or divided into a series of time windows. Each time window can have a period that is less than the overall period that the data was collected over. Each time window includes data that was collected for the different input channels or variables (for each user of the population).

In this embodiment, each time window can include a discrete segment of the discrete glucose measurement data 610, contextual data 620 from the source of user activity data and contextual data 630. Each time window can then be processed at the population learning processor 640 to generate the optimized window population model 670 corresponding to a particular time window. As part of the learning process, the population learning processor 640 can perform various processing tasks described above with reference to FIG. 5 except that the tasks are performed on data taken over a particular time window to generate the optimized window population model 670. Each instance of the optimized window population model 670 performs inference on a discrete specified time segment of the input data. For example, in some embodiments, if a window period of two hours is considered, the optimized window population model would be the model that performs best (e.g., lowest error) for input data segmented to two hour periods.

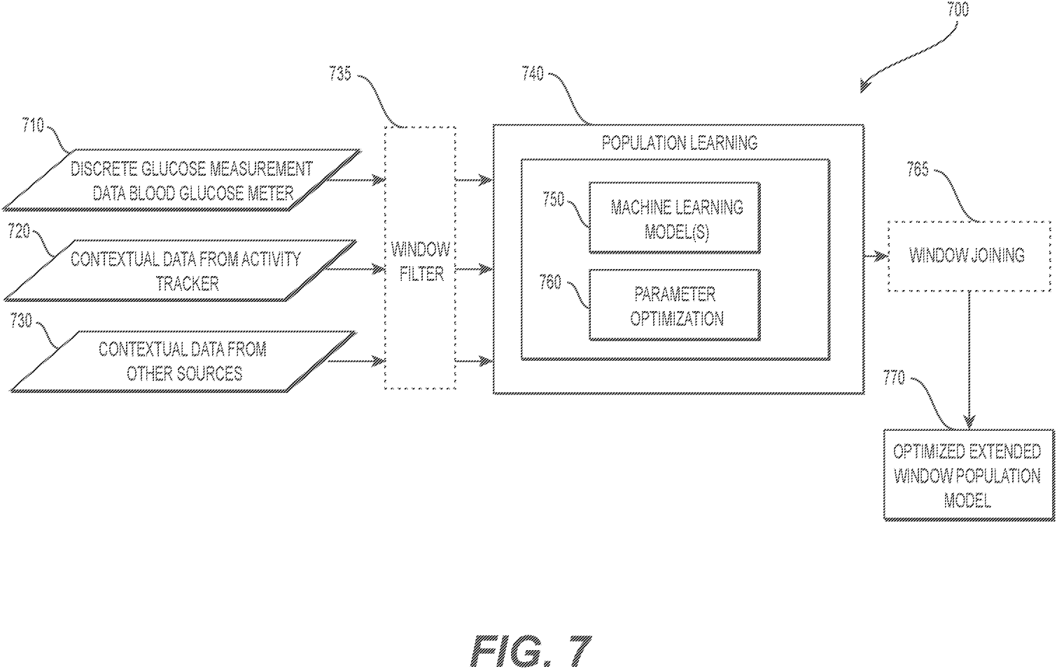

FIG. 7 is a block diagram of another windowed machine learning system 700 for generating an optimized window population model 770 for estimating glucose values for a population of users or patients in accordance with the disclosed embodiments. The structure of the windowed machine learning system 700 is similar to the structure of the windowed machine learning systems 500, 600 described above with reference to FIGS. 5 and 6, respectively. As such, components or blocks shown in FIGS. 5 and 6 will not be described in detail again in conjunction with FIG. 7.

As in FIGS. 5 and 6, data for multiple users is collected that can include, but is not limited to, discrete glucose measurement data 710 from the blood glucose meter, contextual data 720 from the source of user activity data, and contextual data 730 from the other sources. Prior to providing the data to the population learning processor 740 for processing, the data is provided to a window filter 735 that can split the data into different time windows. Each time window can include a discrete segment of the discrete glucose measurement data 710, contextual data 720 from the source of user activity data and contextual data 730. Data from each time window can then be processed at the population learning processor 740 to generate an output (e.g., set of estimated glucose values) corresponding to a particular time window, as described above with reference to FIG. 6. As part of the learning process, the population learning processor 740 can perform various processing tasks described above with reference to FIG. 5 except that the tasks are performed on data taken over a particular time window to generate the optimized window population model.

In this embodiment, a window joining processor 765 is provided that can join sets of the estimated glucose values corresponding to any number of time windows to generate a joined set of estimated glucose values over any number of time windows. For example, in some embodiments, estimated blood glucose sequences over each time window can be stored, and then be sequentially linked together (e.g., assembled/concatenated) at a later time. For example, in some embodiments, estimated blood glucose sequences over each time window can be stored, and when building the model, the stored outputs corresponding to each of the time windows can then be sequentially linked together (e.g., assembled/concatenated) to create the estimation model for the population of users. For example, in some embodiments, if a two hour window filter is considered, the output of the population model (estimated glucose values) would be limited to two hours, as such to generate a longer period output, multiple outputs from multiple windows may be joined. For instance, three two-hour outputs may be joined to generate a six hour model output. In some embodiments, the window joining step takes output of the previous window’s estimated glucose response to initialize the subsequent window. This may be done by replacing or modifying one of the inputs to contain a segment of, or the entire, estimated glucose response output personalized learning step applied to the previous window.

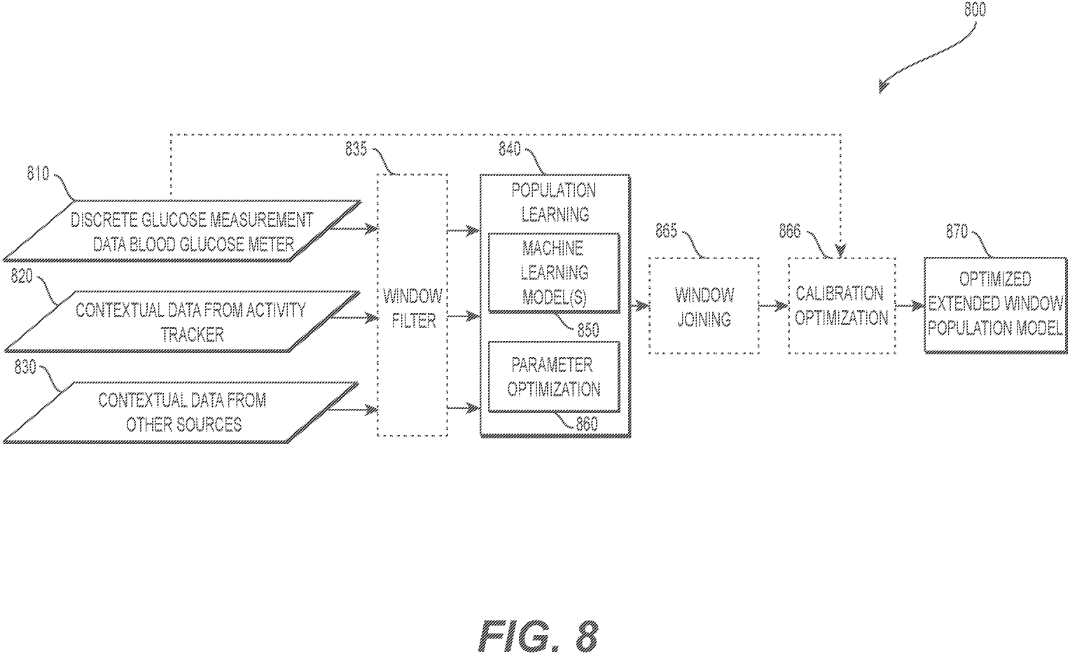

FIG. 8 is a block diagram of another windowed machine learning system 800 for generating an optimized window population model 870 for estimating glucose values for a population of users or patients in accordance with the disclosed embodiments. The structure of the windowed machine learning system 800 is similar to the structure of the windowed machine learning system 500, 600, 700 described above with reference to FIGS. 5-7. As such, components or blocks shown in FIGS. 5-7 will not be described in detail again in conjunction with FIG. 8.

As in FIGS. 5-7, data for multiple users is collected that can include, but is not limited to, discrete glucose measurement data 810 from the blood glucose meter, contextual data 820 from the source of user activity data, and contextual data 830 from the other sources. Prior to providing the data to the population learning processor 840 for processing, the data is provided to a window filter 835 that can split the data into different time windows. Each time window can include a discrete segment of the discrete glucose measurement data 810, contextual data 820 from the source of user activity data and contextual data 830. Data from each time window can then be processed at the population learning processor 840 to generate an optimized window population model corresponding to a particular time window, as described above with reference to FIG. 6. As part of the learning process, the population learning processor 840 can perform various processing tasks described above with reference to FIG. 5 except that the tasks are performed on data taken over a particular time window to generate an output (e.g., set of estimated glucose values) as described with reference to FIG. 6. As described with reference to FIG. 7, a window joining processor 865 can join sets of the estimated glucose values corresponding to any number of time windows to generate a joined set of estimated glucose values over any number of time windows. For example, in some embodiments, estimated blood glucose sequences over each time window can be stored, and then be sequentially linked together (e.g., assembled/concatenated) at a later time outputs. In some embodiments, the window joining step takes output of the previous window’s estimated glucose response to initialize the subsequent window. This may be done by replacing or modifying one of the inputs to contain a segment of, or the entire, estimated glucose response output personalized learning step applied to the previous window.

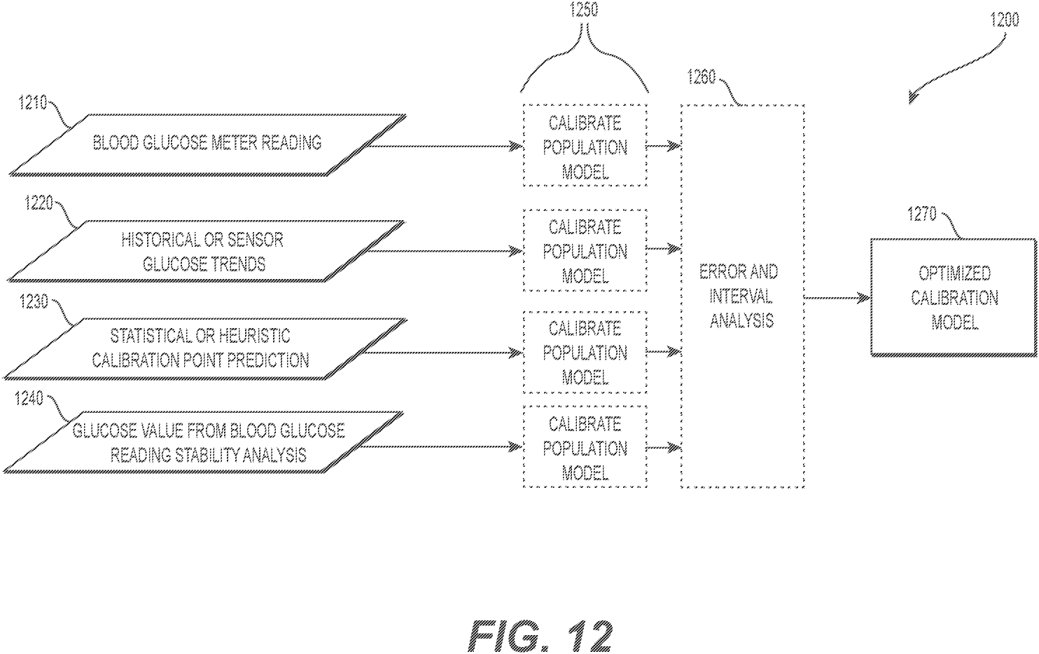

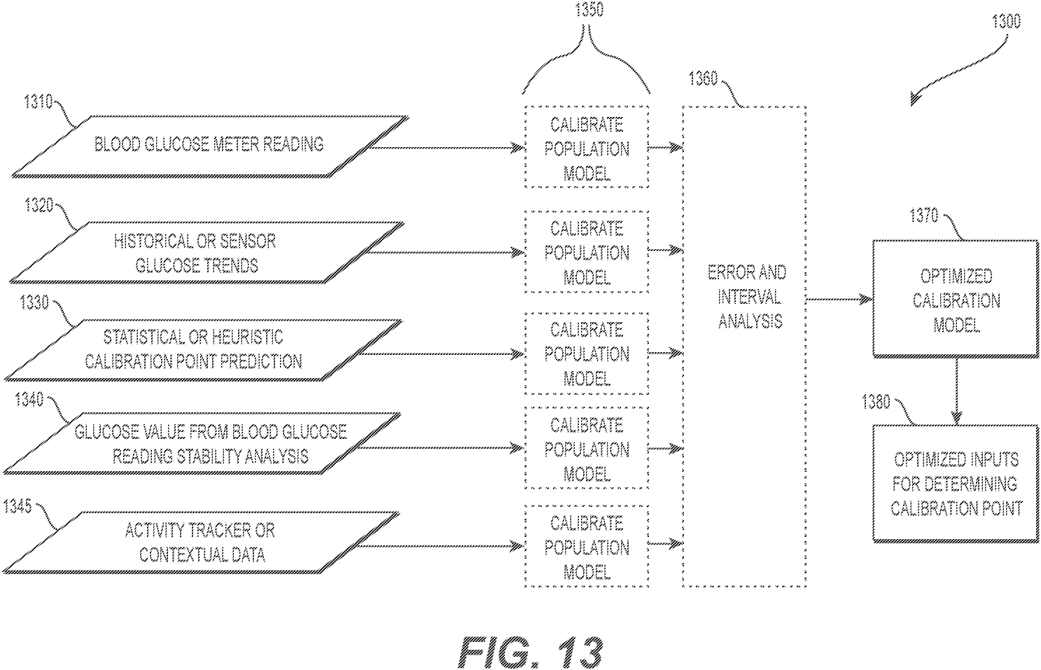

In this non-limiting embodiment, the optimized window population model (described above with reference to FIG. 7) can be processed further by a calibration optimization processor 866 using the discrete glucose measurement data 810 to generate an optimized window population model 870 that has optimized calibration intervals and/or calibration error (e.g., longer intervals between calibration and/or reduced calibration error). The calibration optimization processor 866 can optimize calibration characteristics such as calibration interval and calibration error by determining which calibration population model will give the provide the best compromise between low calibration error and long calibration interval. In general, the longer the interval the better because it is undesirable to calibrate frequently. The calibration intervals of the optimized window population model 870 can be optimized by using the discrete glucose measurement data 810 to adjust the population model output to yield a more accurate estimated glucose. For instance, the adjustment may be made on an interval of minutes, hours, weeks or months. The specific adjustment interval needs may be determined by testing each calibration interval and selecting the population and calibration interval that provides the most accurate glucose estimate. In some embodiments, the calibration interval processor need not rely exclusively on blood glucose meter data, but may in substitute, or in addition, include data from other sources. Processing performed by the calibration optimization processor 866 will be described in greater detail below with reference to FIGS. 12 and 13.

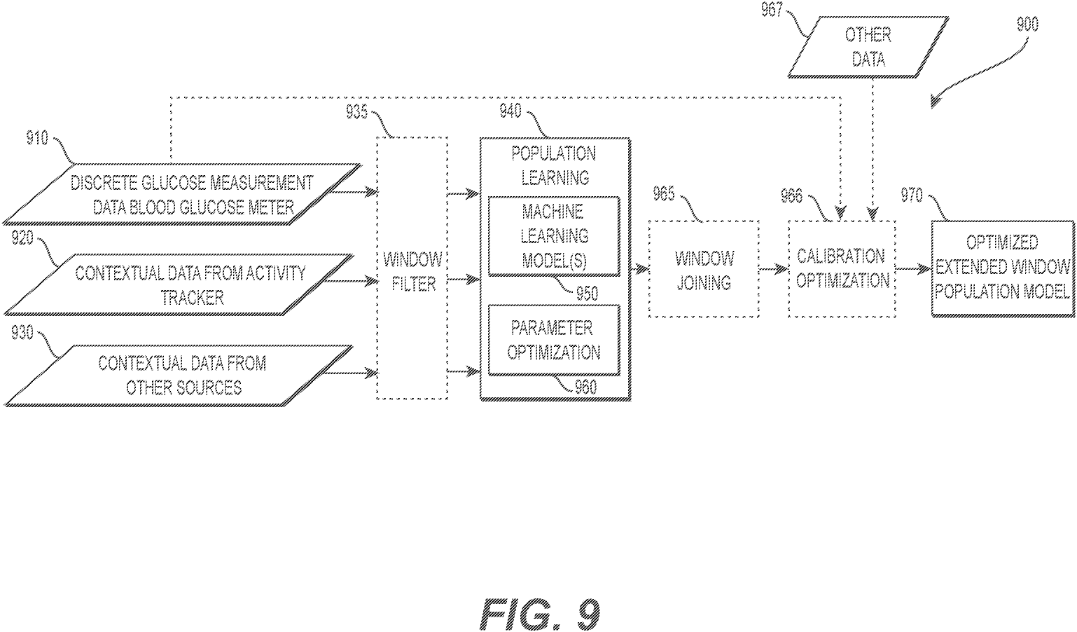

FIG. 9 is a block diagram of another windowed machine learning system 900 for generating an optimized window population model 970 for estimating glucose values for a population of users or patients in accordance with the disclosed embodiments. The structure of the windowed machine learning system 900 is similar to the structure of the windowed machine learning systems 500, 600, 700, 800 described above with reference to FIGS. 5-8. As such, components or blocks shown in FIGS. 5-8 will not be described in detail again in conjunction with FIG. 9.

As in FIGS. 5-8, data for multiple users is collected that can include, but is not limited to, discrete glucose measurement data 910 from the blood glucose meter, contextual data 920 from the source of user activity data, and other contextual data 930 from the other sources. Prior to providing the data to the population learning processor 940 for processing, the data can be provided to a window filter 935 that can split the data into different time windows. Each time window can include a discrete segment of the discrete glucose measurement data 910, contextual data 920 from the source of user activity data and other contextual data 930. Data from each time window can then be processed at the population learning processor 940 to generate an output (e.g., set of estimated glucose values) corresponding to a particular time window, as described above with reference to FIG. 6. As part of the learning process, the population learning processor 940 can perform various processing tasks described above with reference to FIG. 5 except that the tasks are performed on data taken over a particular time window to generate an output (e.g., set of estimated glucose values). As described with reference to FIG. 7, a window joining processor 965 can join sets of the estimated glucose values corresponding to any number of time windows to generate a joined set of estimated glucose values over any number of time windows. For example, in some embodiments, estimated blood glucose sequences over each time window can be stored, and then be sequentially linked together (e.g., assembled/concatenated) at a later time. In some embodiments, the window joining step takes output of the previous window’s estimated glucose response to initialize the subsequent window. This may be done by replacing or modifying one of the inputs to contain a segment of, or the entire, estimated glucose response output personalized learning step applied to the previous window.

In this embodiment, the optimized window population model (as described above with reference to FIG. 7) can be processed further by a calibration optimization processor 966 using the discrete glucose measurement data 910 and/or other data 967 to generate an optimized window population model (as described above with reference to FIG. 8) that has optimized calibration intervals and/or calibration error (e.g., longer intervals between calibration and/or reduced calibration error).

In this embodiment, the other data 967 can include, for example, one or more past blood glucose meter readings, past glucose sensor data, time and blood glucose value that has the least daily, weekly or monthly variance, past data from any one of the model inputs, etc. Utilizing the other data 967 in alone or in conjunction with the discrete glucose measurement data 910 can improve the accuracy and/or calibration interval of the accuracy of the calibration interval of the optimized window population model that is output by the calibration optimization processor 966. As described above with respect to the calibration optimization processor 866 of FIG. 8, other data 967 from other sources may be used in conjunction with or in place of discrete glucose measurement data 910. For instance, other data 967 may provide enough information to provide an accurate calibration method, without the need for invasive blood glucose meter readings. In another instance, to reduce the frequency (lengthen the interval) of blood glucose meter readings, other data 967 may be used to assist the calibration model to maintain satisfactory performance of the population model accuracy. Processing performed by the calibration optimization processor 966, and the variety of inputs or data it can use in place of historical and blood glucose meter data, will be described in greater detail below with reference to FIGS. 12 and 13.

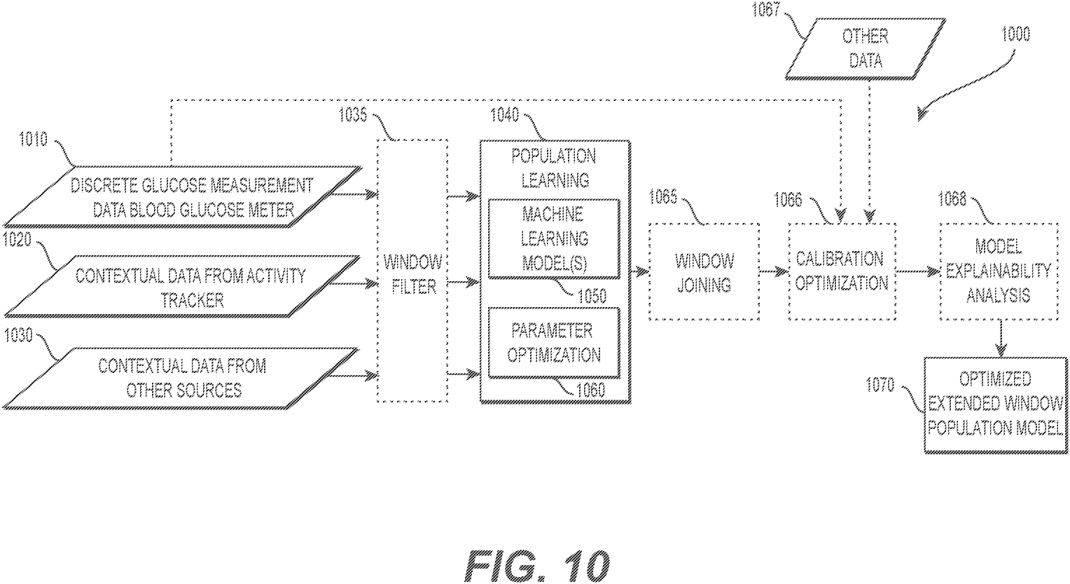

FIG. 10 is a block diagram of another windowed machine learning system 1000 for generating an optimized extended window population model 1070 for estimating glucose values for a population of users or patients in accordance with the disclosed embodiments. The structure of the windowed machine learning system 1000 is similar to the structure of the windowed machine learning systems 500, 600, 700, 800, 900 described above with reference to FIGS. 5-9. As such, components or blocks shown in FIGS. 5-9 will not be described in detail again in conjunction with FIG. 10.

As in FIGS. 5-9, data for multiple users is collected that can include, but is not limited to, discrete glucose measurement data 1010 from the blood glucose meter, contextual data 1020 from the source of user activity data, and contextual data 1030 from the other sources. Prior to providing the data to the population learning processor 1040 for processing, the data can be provided to a window filter 1035 that can split the data into different time windows. Each time window can include a discrete segment of the discrete glucose measurement data 1010, contextual data 1020 from the source of user activity data and contextual data 1030. Data from each time window can then be processed at the population learning processor 1040 to generate an optimized window population model corresponding to a particular time window, as described above with reference to FIG. 6. As part of the learning process, the population learning processor 1040 can perform various processing tasks described above with reference to FIG. 5 except that the tasks are performed on data taken over a particular time window to generate an output (e.g., set of estimated glucose values) as described with reference to FIG. 6. As described with reference to FIG. 7, a window joining processor 1065 can join sets of the estimated glucose values corresponding to any number of time windows to generate a joined set of estimated glucose values over any number of time windows. For example, in some embodiments, estimated blood glucose sequences over each time window can be stored, and then be sequentially linked together (e.g., assembled/concatenated) at a later time. In some embodiments, the window joining step takes output of the previous window’s estimated glucose response to initialize the subsequent window. This may be done by replacing or modifying one of the inputs to contain a segment of, or the entire, estimated glucose response output personalized learning step applied to the previous window. As explained with reference to FIGS. 8 and 9, the optimized window population model (generated in FIG. 7) can be processed further by a calibration optimization processor 1066 using discrete glucose measurement data 1010 and/or other data 1067 to generate an optimized window population model (described above with reference to FIG. 8) that has optimized calibration intervals and/or calibration error (e.g., longer intervals between calibration and/or reduced calibration error).

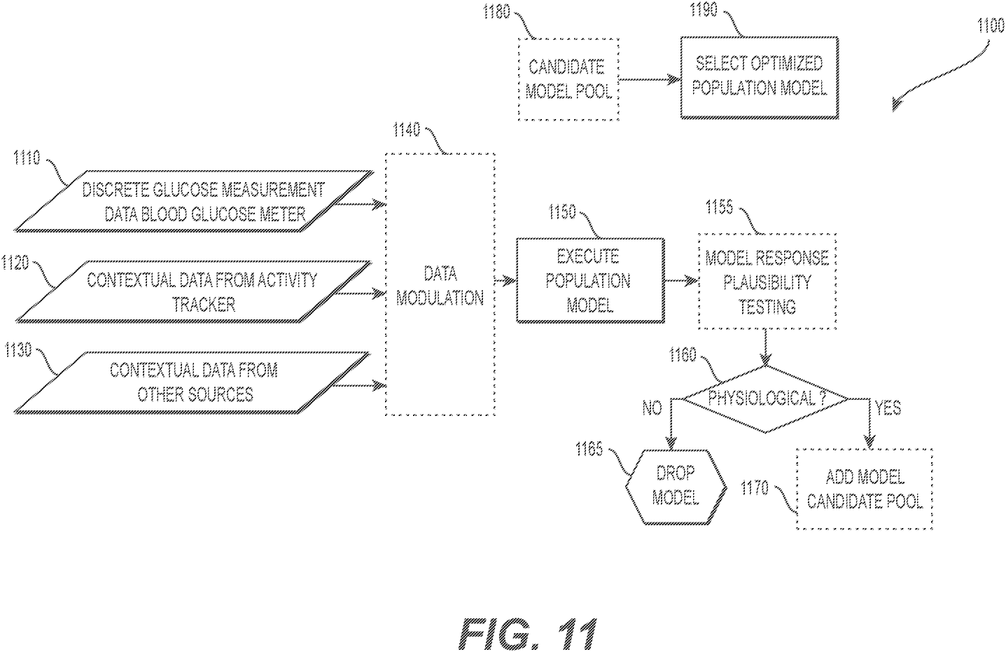

In this embodiment, the optimized window population model (described above with reference to FIG. 9) that is output by the calibration optimization processor 1066 can be processed further by a model explainability analysis processor 1068 to generate an optimized extended window population model 1070 that provides a physiologically consistent response to one or more of the model inputs. For example, the typical human physiological response to food intake is a rise in blood glucose levels. Therefore, each candidate population model may be tested to ensure the response is at least directionally similar, or similar in magnitude, to what is observed clinically, medically, or physiologically. As will be described in greater detail below, the model explainability analysis processor 1068 can modulate or simulate the inputs and compare the observed population model output (or response) to a reference or standard. This comparison may indicate the plausibility and explainability of the model response. This process may remove from consideration all models that fail to respond in an understandable manner. The model explainability analysis processor 1068 can select and output or generate an optimized extended window population model 1070. The processing performed by the model explainability analysis processor 1068 will now be described in greater detail below with reference to FIG. 11.

FIG. 11 is a block diagram of a model explainability analysis processor 1100 for selecting an optimized population model for estimating glucose values for a population of users or patients in accordance with the disclosed embodiments. The model explainability analysis processor 1100 can select an optimized population model from a pool 1180 of candidate population models. The selected optimized population model can then be used as the basis for further training and personalized to generate a personalized model for estimating glucose values for a particular user or patient.

During this part of the workflow process data for multiple users can be collected for processing. In this example, the data that is collected can include, but is not limited to, discrete glucose measurement data 1110 from the blood glucose meter, contextual data 1120 from the source of user activity data, and contextual data 1130 from the other sources.

The discrete glucose measurement data 1110 from the blood glucose meter, contextual data 1120 from the source of user activity data and contextual data 1130 from the other sources can then be processed at a data modulation processor 1140 to generate a modulated form of the data. For example, in some embodiments, the input data can be modulated (modified) or simulated to generate a change in the content of one or more inputs. The modulated data is executed by a population model to generate the estimated glucose output. In the model response testing step, the output estimated glucose output is compared to a reference or standard that represents the typical directionality or magnitude of a human physiological response to a similar input data modulation. The reference or standard can be, not limited to, documented or general know how of what is typically observed clinically, medically, or physiologically. For example, the typical human physiological response to insulin is a drop in blood glucose levels. Modulation of an insulin input by reduction of the magnitude of the insulin level, in a physiologically accurate model, should result in a lower estimated glucose output.

At 1150 through 1160, the model explainability analysis processor 1100 can then perform processing to evaluate each population model of a number of population models to determine which of those population models should be added to a pool 1180 of candidate population models, and then select (at 1190) an optimized population model from the pool 1180 of candidate population models as an optimized population model (e.g., an optimized window population model, an optimized window population model, etc.). At 1150, the model explainability analysis processor 1100 selects a population model to execute and applies the modulated form of the data to the selected population model to generate a response to the modulated form of the data. At 1155, the model explainability analysis processor 1100 can perform various tests that help assess or evaluate whether the response (that was generated at 1150) is physiologically plausible. At 1160, the model explainability analysis processor 1100 can then analyze or evaluate the test results generated at 1155 to determine whether the selected population model’s response is a plausible physiological response. When the model explainability analysis processor 1100 determines (at 1160) that the test results generated at 1155 indicate that the selected population model’s response is not a plausible physiological response, the model explainability analysis processor 1100 drops the selected population model from further consideration.

By contrast, when the model explainability analysis processor 1100 determines (at 1160) that the test results generated at 1155 indicate that the selected population model’s response is a plausible physiological response, the model explainability analysis processor 1100 adds the selected population model to a pool 1180 of candidate population models at 1170. At 1190, the model explainability analysis processor 1100 can select an optimized population model from the pool 1180 of candidate population models. The selected optimized population model can then be used as the basis for further training and personalized to generate a personalized model for estimating glucose values for a particular user or patient.

Referring again to FIGS. 8 through 10, the calibration optimization processor 866, 966, 1066 can identify a calibration model that provides the best performance, and also can identify the best type of input to use for developing a calibration model (e.g., selecting the best calibration approach). The processing performed by the calibration optimization processor 866, 966, 1066 will now be described in greater detail below with reference to FIGS. 12 and 13.

FIG. 12 is a block diagram of a calibration optimization processor 1200 for selecting an optimized calibration model 1270 in accordance with the disclosed embodiments. The calibration optimization processor 1200 can select an optimized calibration model 1270 to be used as the basis for calibrating a population model used for estimating glucose values for a population of users.

During this part of the workflow process data can be collected from different potential calibration sources 1210, 1220, 1230, 1240 for processing and evaluation. In this example, the data that is collected can include, but is not limited to, discrete glucose measurement data 2010 collected from a blood glucose meter, historical or sensor glucose trend data 1220, statistical or heuristic calibration point prediction data 1230, and glucose value data 1240 from blood glucose reading stability analysis. Historical or sensor glucose trends can include, but not limited to, glucose values from the past, several days of glucose sensor data, time of day point(s) where glucose is less or least variables. Statistical or heuristic calibration point prediction can include, but not limited to, a statistical, empirical, or rule-based method that estimates or predicts the glucose value at a given point in time, which may be used to calibrate the population model. Glucose value from blood glucose reading stability analysis, can include, but not limited to, an analysis of variability of glucose on an hourly, daily, weekly, or monthly basis. The glucose value that is most stable, less or least variable compared to other periods of time, may be selected to calibrate the population model. Each source of data 1210, 1220, 1230, 1240, or combination thereof, can be applied to a calibrate population model processor 1250 to generate a calibration model that is applied to the population model generating a corresponding calibrated population model response. In other words, while FIG. 12 illustrates a single arrow between each source of data 1210, 1220, 1230, 1240 and each calibrate population model processor 1250, it should be appreciated that in other implementations, each source of data 1210, 1220, 1230, 1240 could be linked to each calibrate population model processor 1250.

An error and interval analysis processor 1260 can receive each calibrated population model response. For each calibrated population model response, the error and interval analysis processor 1260 can (1) perform an error analysis and generate an error result that indicates the performance of the calibrated population model response compared to the measured glucose values, and (2) perform an interval analysis to generate an interval result that indicates how often the population model would need to be calibrated with the given input. For each response, the error and interval analysis processor 1260 can then evaluate, the corresponding error result and the corresponding interval result.

At 1270, based on the evaluation of all of the error result/interval result combinations for each type of calibration model, the calibration optimization processor 1200 can compare performance of any given population model for each type of calibration model to determine an optimized calibration model for that given population model. The optimized calibration model can be selected, for example, to minimize error, maximize time between calibrations, and/or reduce user or patient burden. For example, a blood glucose meter reading every few minutes may minimize error but would be burdensome on the user, and as such would have a calibration interval that is too frequent. A blood glucose meter is considered an invasive measurement and thus burdensome to the patient, thus one of the other calibration models based on the alternate input sources that are less invasive or non-invasive may be preferred. The tradeoff between error, calibration interval, and patient burden, may be based on an objective function, clinical criteria, or product, regulatory or business requirements.

FIG. 13 is a block diagram of a calibration optimization processor 1300 for selecting optimal type(s) of inputs to be used for developing an optimized calibration model in accordance with the disclosed embodiments. The calibration optimization processor 1300 can select one or more inputs to use with an optimized calibration model when calibrating any given population model used for estimating glucose values for a population of users.

During this part of the workflow process data can be collected from different potential calibration sources 1310, 1320, 1330, 1340, 1345 for processing and evaluation. In this example, the data that is collected can include, but is not limited to, discrete glucose measurement data 2010 collected from a blood glucose meter, historical or sensor glucose trend data 1320, statistical or heuristic calibration point prediction data 1330, glucose value data 1340 from blood glucose reading stability analysis, and contextual data 1345 from the source of user activity data (e.g., an activity tracker), and/or contextual data 1345 from the other sources such as nutritional information about meals consumed by a user, insulin delivered to the user by an insulin infusion device of the user, etc. Historical or sensor glucose trends can include, but not limited to, glucose values from the past, several days of glucose sensor data, time of day point(s) where glucose is less or least variables. Statistical or heuristic calibration point prediction can include, but not limited to, a statistical, empirical, or rule-based method that estimates or predicts the glucose value at a given point in time, which may be used to calibrate the population model. Glucose value from blood glucose reading stability analysis, can include, but not limited to, an analysis of variability of glucose on an hourly, daily, weekly, or monthly basis. The glucose value that is most stable, less or least variable compared to other periods of time, may be selected to calibrate the population model. Each source of data 1310, 1320, 1330, 1340, 1345, or combination thereof, can be applied to a calibrate population model processor 1350 to generate a calibration model that is applied to the population model generating a corresponding calibrated population model response. In other words, while FIG. 13 illustrates a single arrow between each source of data 1310, 1320, 1330, 1340 and each calibrate population model processor 1350, it should be appreciated that in other implementations, each source of data 1310, 1320, 1330, 1340 could be linked to each calibrate population model processor 1350.

An error and interval analysis processor 1360 can receive each calibrated population model response. For each calibrated population model response, the error and interval analysis processor 1360 can (1) perform an error analysis and generate an error result that indicates the performance of the calibrated population model response compared to the measured glucose values, and (2) perform an interval analysis to generate an interval result that indicates how often the population model would need to be calibrated with the given input. For each response, the error and interval analysis processor 1360 can then evaluate, the corresponding error result and the corresponding interval result.

At 1370, based on the evaluation of all of the error result/interval result combinations for each type of calibration model, the calibration optimization processor 1300 can compare performance of any given population model for each type of calibration model to determine an optimized calibration model for that given population model. The optimized calibration model can be selected, for example, to minimize error, maximize time between calibrations, and/or reduce user or patient burden. For example, a blood glucose meter reading every few minutes may minimize error but would be burdensome on the user, and as such would have a calibration interval that is too frequent. A blood glucose meter is considered an invasive measurement and thus burdensome to the patient, thus one of the other calibration models based on the alternate input sources that are less invasive or non-invasive may be preferred. The tradeoff between error, calibration interval, and patient burden, may be based on an objective function, clinical criteria, or product, regulatory or business requirements.

In this embodiment, once the optimized calibration model is determined (at 1370), the calibration optimization processor 1300 can determine or select at 1380, based on the evaluation of all of the error result/interval result combinations for each type of input, optimal type(s) of inputs to be used with that optimized calibration model (from 1370) when it is used for calibrating any given population model. As such, once an optimized calibration model has been determined (at 1370), the calibration optimization processor 1300 can select (at 1380) one or more types of the inputs 1310, 1320, 1330, 1340, 1345 to use with that optimized calibration model.

As described above, at least one of the population models can be selected, and personalized model learning processes can be performed to personalize a selected population model for each particular user. Various embodiments of personalized model learning processes that can be performed will now be described below with reference to FIGS. 14 through 21. In those descriptions, like reference numbers refer to similar elements throughout the figures.

FIG. 14 is a block diagram of a machine learning system 1400 for generating an optimized personal model 1470 for estimating glucose values that are personalized for a particular user or patient in accordance with the disclosed embodiments. During this part of the workflow process data for a particular user is collected for processing. In this example, the data that is collected can include, but is not limited to, discrete glucose measurement data 1410 from the blood glucose meter or other sensor arrangement that provides discrete glucose measurements from the user, contextual data 1420 about the user from the source of user activity data (e.g., an activity tracker), and contextual data 1430 about the user from the other sources such as nutritional information about meals consumed by a user, insulin delivered to the user by an insulin infusion device of the user.

Data for each of the inputs can be collected for the particular user over a time period. Stated differently, data for the particular user can be collected for different input channels. Each of the different input channels can represent a different variable being measured for that user. In some embodiments, the different input channels can include (1) time of day, (2) a blood glucose level, (3) a blood glucose (BG) measurement from a sensor such as a CGM device, activity data including (4) heart rate (beats per minute), (5) METs and (6) number of steps, (7) active insulin, (8) carbohydrates on board, etc.

In some embodiments, the discrete glucose measurement data 1410, contextual data 1420 from the source of user activity data and contextual data 1430 from the other sources can then be processed at a personalized learning processor 1440 along with a particular population model 1405 to train or adapt the particular population model 1405 and thus generate the optimized personal model 1470 that is tailored to the particular user or patient. In some embodiments, the particular population model 1405 serves as the machine learning model 1450 and learns based on the inputs 1410, 1420, 1430 such that the particular population model 1405 is transformed (e.g., re-trained and optimized/personalized to the user) to become the optimized personal model 1470 for estimating glucose values that are personalized for a particular user or patient. In other embodiments, the particular population model 1405 is one model of an ensemble of the machine learning models 1450, and the ensemble of machine learning models 1450 learns based on the inputs 1410, 1420, 1430 such that the ensemble is transformed to become the optimized personal model 1470 for estimating glucose values that are personalized for a particular user or patient. In this example, as part of the personalized learning process, the personalized learning processor 1440 can perform various processing tasks with respect to the discrete glucose measurement data 1410 from the blood glucose meter, contextual data 1420 from the source of user activity data and contextual data 1430 from the other sources to train or adapt the particular population model 1405 to generate the optimized personal model 1470. For example, the personalized learning processor 1440 can apply various machine learning models 1450 including the particular population model 1405, to the discrete glucose measurement data 1410, contextual data 1420 from the source of user activity data and the other contextual data 1430 from the other sources to train or adapt the particular population model 1405. In another embodiment, the particular population model 1405 is combined with a separate personalized model to create an ensemble of population and personalized models that constitute a single optimized personal model. In another embodiment, the particular population model 1405 maybe disregarded and skipped over (e.g., given a zero or near zero weight), in which case the personalized learning processor 1440 can apply other various machine learning models 1450 to generate the optimized personal model 1470 that is tailored to the particular user or patient.

The personalized learning processor 1440 can also perform various parameter optimizations based on the discrete glucose measurement data 1410 from the blood glucose meter, contextual data 1420 from the source of user activity data and contextual data 1430 from the other sources to further train the particular population model 1405. For example, one or more machine learning or deep learning model(s) can learn the transfer function or mapping from a series of inputs to a glucose measurement from a glucose sensor. This mapping can be on any time interval such that the model estimates anywhere from a single glucose value to a series of glucose values continuous in time. Each machine learning or deep learning model may have one or more parameters that need to be identified to specify the mathematical transfer function. Parameter optimization generally includes an objective function that must be minimized. The objective function measures the mathematical agreement between the estimated output of the model with the actual measured glucose values. Parameters are typically interactively adjusted until the objective function is optimized. The optimization process terminates once the level of agreement reaches a desired threshold or no longer improves.

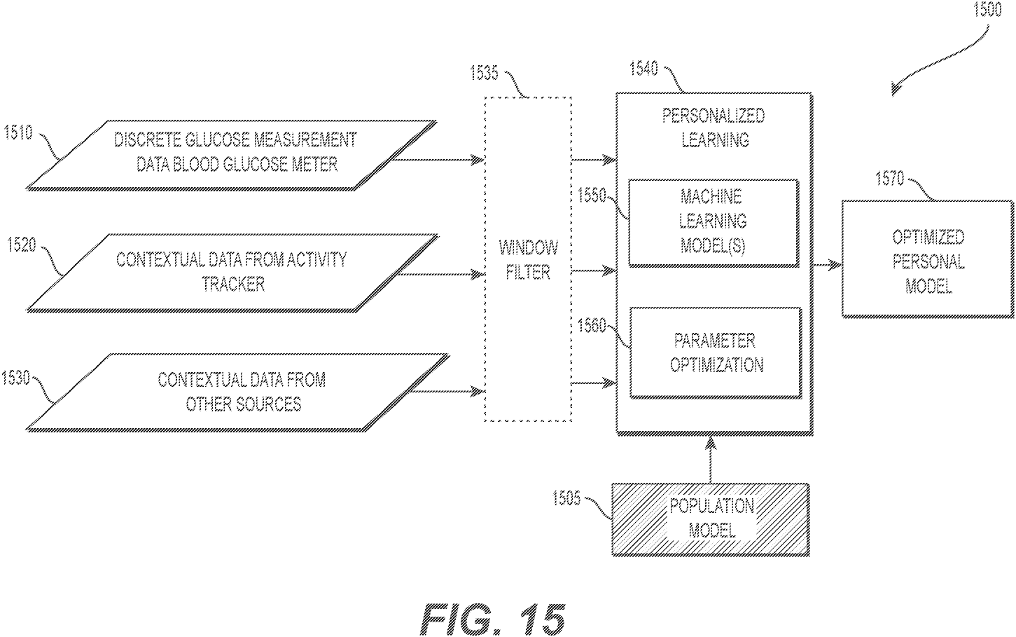

FIG. 15 is a block diagram of a windowed machine learning system 1500 for generating an optimized personal model 1570 for estimating glucose values that are personalized for a particular user or patient in accordance with the disclosed embodiments. The structure of the windowed machine learning system 1500 is similar to the structure of the machine learning system 1400 described above with reference to FIG. 14. As such, components or blocks shown in FIG. 14 will not be described in detail again in conjunction with FIG. 15.

As described above with reference to FIG. 14, data for the particular user is collected for processing that can include, but is not limited to, discrete glucose measurement data 1510 from the blood glucose meter, contextual data 1520 from the source of user activity data, and contextual data 1530 from the other sources. However, in this embodiment, prior to providing the data to the personalized learning processor 1540 for processing, the data is provided to a window filter 1535. The window filter 1535 can split the data into different time windows. In other words, the collected data can be sequentially split or divided into a series of time windows. Each time window can have a period that is less than the overall period that the data was collected over. Each time window includes data that was collected for the different input channels or variables (for each user of the personalized).

In this embodiment, each time window can include a discrete segment of the discrete glucose measurement data 1510, contextual data 1520 from the source of user activity data and contextual data 1530 for the particular user. Each time window can then be processed at the personalized learning processor 1540 to generate the optimized personal model 1570 corresponding to a particular time window. As part of the learning process, the personalized learning processor 1540 can perform various processing tasks described above with reference to FIG. 14 except that the tasks are performed on data taken over a particular time window to generate the optimized personal model 1570. Each instance of the optimized personal model 1570 performs inference on a discrete specified time segment of the input data. For example, in some embodiments, if a window period of two hours is considered, the optimal personalized model can be the model that performs best (e.g., lowest error) for input data segmented to two hour periods.

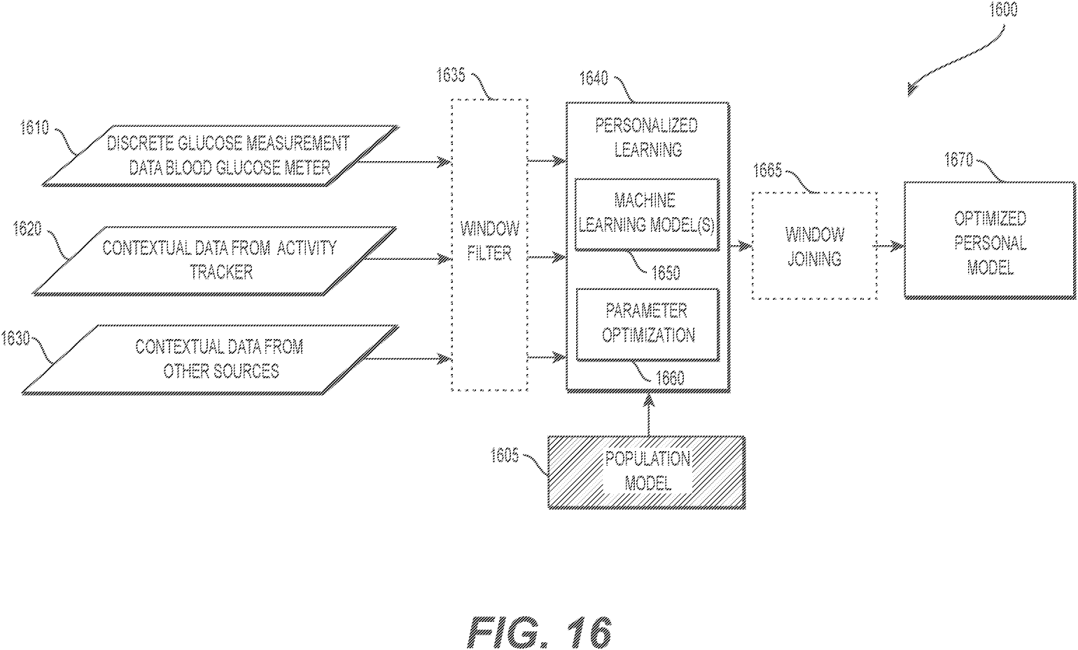

FIG. 16 is a block diagram of another windowed machine learning system 1600 for generating an optimized personal model 1670 for estimating glucose values that are personalized for a particular user or patient in accordance with the disclosed embodiments. The structure of the windowed machine learning system 1600 is similar to the structure of the windowed machine learning system 1500 described above with reference to FIGS. 14 and 15. As such, components or blocks shown in FIGS. 14 and 15 will not be described in detail again in conjunction with FIG. 16.

As in FIGS. 14 and 15, data for the particular user is collected that can include, but is not limited to, discrete glucose measurement data 1610 from the blood glucose meter, contextual data 1620 from the source of user activity data, and contextual data 1630 from the other sources. Prior to providing the data to the personalized learning processor 1640 for processing, the data is provided to a window filter 1635 that can split the data into different time windows. Each time window can include a discrete segment of the discrete glucose measurement data 1610, contextual data 1620 from the source of user activity data and contextual data 1630. Data from each time window can then be processed at the personalized learning processor 1640 to generate an output (e.g., set of estimated glucose values) corresponding to a particular time window, as described above with reference to FIG. 15. As part of the learning process, the personalized learning processor 1640 can perform various processing tasks described above with reference to FIGS. 14 and 15 except that the tasks are performed on data taken over a particular time window to generate the optimized personal model 1670.

In this embodiment, a window joining processor 1665 is provided that can join sets of the estimated glucose values corresponding to any number of time windows to generate a joined set of estimated glucose values over any number of time windows. For example, in some embodiments, estimated blood glucose sequences over each time window can be stored, and then be sequentially linked together (e.g., assembled/concatenated) at a later time. For example, in some embodiments, estimated blood glucose sequences over each time window can be stored, and the stored outputs corresponding to each of the time windows can then be sequentially linked together (e.g., assembled/concatenated). In some embodiments, the window joining step takes output of the previous window’s estimated glucose response to initialize the subsequent window. This may be done by replacing or modifying one or more of the inputs to contain a segment of, or the entire, estimated glucose response output personalized learning step applied to the previous window.

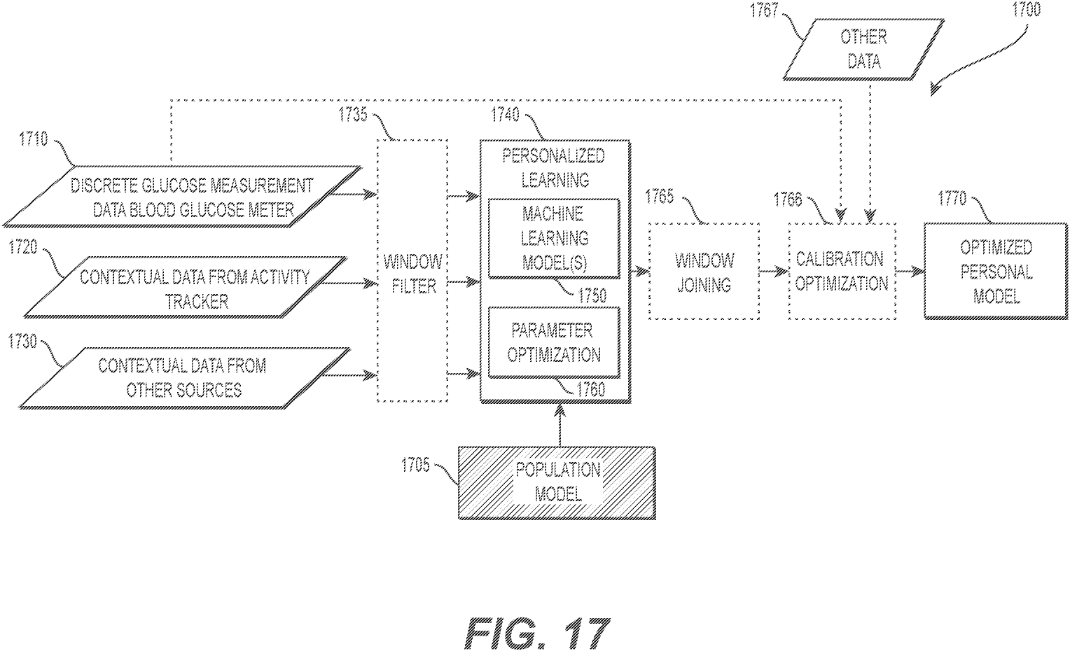

FIG. 17 is a block diagram of another windowed machine learning system 1700 for generating an optimized personal model 1770 for estimating glucose values that are personalized for a particular user or patient in accordance with the disclosed embodiments. The structure of the windowed machine learning system 1700 is similar to the structure of the windowed machine learning system 1600 described above with reference to FIGS. 14-16. As such, components or blocks shown in FIGS. 14-16 will not be described in detail again in conjunction with FIG. 17 unless they perform differently in the embodiment of FIG. 17.

As in FIGS. 14-16, data for the particular user is collected that can include, but is not limited to, discrete glucose measurement data 1710 from the blood glucose meter, contextual data 1720 from the source of user activity data, and contextual data 1730 from the other sources. Prior to providing the data to the personalized learning processor 1740 for processing, the data is provided to a window filter 1735 that can split the data into different time windows. Each time window can include a discrete segment of the discrete glucose measurement data 1710, contextual data 1720 from the source of user activity data and contextual data 1730. Data from each time window can then be processed at the personalized learning processor 1740 to generate an optimized personal model 1570 corresponding to a particular time window, as described above with reference to FIG. 15. As part of the learning process, the personalized learning processor 1740 can perform various processing tasks described above with reference to FIG. 14 except that the tasks are performed on data taken over a particular time window to generate an output (e.g., set of estimated glucose values) as described with reference to FIG. 15. As described with reference to FIG. 16, a window joining processor 1765 can join sets of the estimated glucose values corresponding to any number of time windows to generate a joined set of estimated glucose values over any number of time windows. For example, in some embodiments, estimated blood glucose sequences over each time window can be stored, and then be sequentially linked together (e.g., assembled/concatenated) at a later time. In some embodiments, the window joining step takes output of the previous window’s estimated glucose response to initialize the subsequent window. This may be done by replacing or modifying one of the inputs to contain a segment of, or the entire, estimated glucose response output personalized learning step applied to the previous window.

In this embodiment, the optimized personal model 1670 (generated in FIG. 16) can be processed further by a calibration optimization processor 1766 using the discrete glucose measurement data 1710 and/or other data 1767 to generate an optimized personal model 1770 that has calibration intervals optimized. The calibration intervals of the optimized personal model 1770 can be optimized by using the discrete glucose measurement data 1710. In some embodiments, the optimized personal model 1670 (generated as described above with reference to FIG. 16) can be processed further by the calibration optimization processor 1766 using discrete glucose measurement data 1710 to generate the optimized personal model 1770. In other embodiments, calibration optimization processor 1766 can also process other data, such as other data 1767 with or without the discrete glucose measurement data 1710 to generate an optimized personal model 1770. In some embodiments, the other data 1867 can include at least one or more of, for example, one or more past blood glucose meter readings, past glucose sensor data, time and blood glucose value that has the least daily, weekly or monthly variance, past data from any one of the model inputs, etc. Utilizing the other data 1767 in conjunction with the discrete glucose measurement data 1710 can improve the accuracy of the calibration interval of the optimized personal model that is output by the calibration optimization processor 1766. Processing performed by the calibration optimization processor 1766 can be performed as described above with reference to FIGS. 12 and 13.

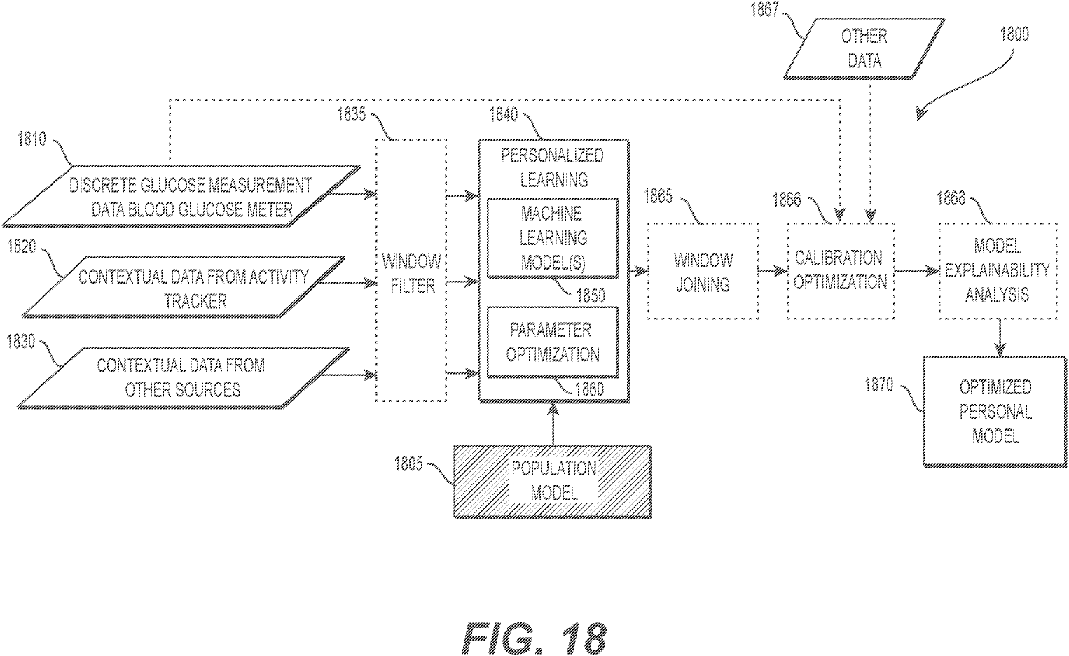

FIG. 18 is a block diagram of another windowed machine learning system 1800 for generating an optimized personal model 1870 for estimating glucose values for a personalized a particular user or patient in accordance with the disclosed embodiments. The structure of the windowed machine learning system 1800 is similar to the structure of the windowed machine learning system 1700 described above with reference to FIGS. 14-17. As such, components or blocks shown in FIGS. 14-17 will not be described in detail again in conjunction with FIG. 18 unless they perform differently in the embodiment of FIG. 18.

As in FIGS. 14-17, data for the particular user is collected that can include, but is not limited to, discrete glucose measurement data 1810 from the blood glucose meter, contextual data 1820 from the source of user activity data, and contextual data 1830 from the other sources. Prior to providing the data to the personalized learning processor 1840 for processing, the data can be provided to a window filter 1835 that can split the data into different time windows. Each time window can include a discrete segment of the discrete glucose measurement data 1810, contextual data 1820 from the source of user activity data and contextual data 1830. Data from each time window can then be processed at the personalized learning processor 1840 to generate an optimized personal model corresponding to a particular time window, as described above with reference to FIG. 15. As part of the learning process, the personalized learning processor 1840 can perform various processing tasks described above with reference to FIG. 14 except that the tasks are performed on data taken over a particular time window to generate the output (e.g., set of estimated glucose values) as described with reference to FIG. 15. As described with reference to FIG. 16, a window joining processor 1865 can join sets of the estimated glucose values corresponding to any number of time windows to generate a joined set of estimated glucose values over any number of time windows. For example, in some embodiments, estimated blood glucose sequences over each time window can be stored, and then be sequentially linked together (e.g., assembled/concatenated) at a later time. In some embodiments, the window joining step takes output of the previous window’s estimated glucose response to initialize the subsequent window. This may be done by replacing or modifying one of the inputs to contain a segment of, or the entire, estimated glucose response output personalized learning step applied to the previous window.

As explained with reference to FIG. 17, in some embodiments, the optimized personal model (generated as described above with reference to FIG. 16) can be processed further by a calibration optimization processor 1866 to generate an optimized personal model (as described above with reference to FIG. 17).

In this embodiment, the optimized personal model that is output by the calibration optimization processor 1866 can then be processed further by the model explainability analysis processor 1868 to generate an optimized personal model 1870 that provides a physiologically consistent response to one or more of the model inputs. For example, the typical human physiological response to food intake is a rise in blood glucose levels. Therefore, each candidate population model may be tested to ensure the response is at least directionally similar, or similar in magnitude, to what is observed clinically, medically, or physiologically. As described with reference to FIGS. 10 and 11, the model explainability analysis processor 1868 can modulate or simulate the inputs and compare the observed personal model output (or response) to a reference or standard. This comparison may indicate the plausibility and explainability of the model response. This process may remove from consideration all models that fail to respond in an understandable manner. The model explainability analysis processor 1868 can select and output or generate an optimized personalized model 1870.

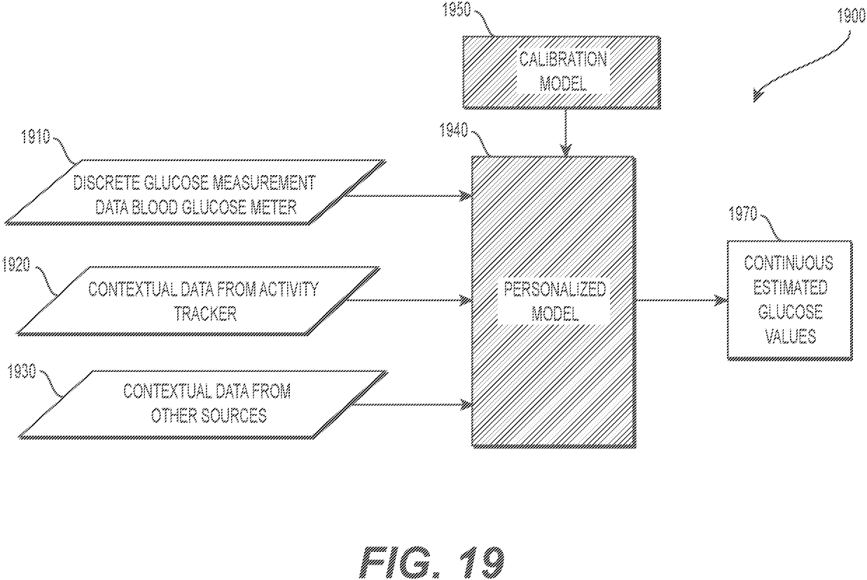

FIG. 19 is a block diagram that illustrates an intermittent CGM system 1900 in accordance with the disclosed embodiments. The system 1900 includes a personalized model 1940 and a calibration model 1950. The personalized model 1940 can refer to any type of personalized model including the optimized and windowed personal models described above. Once the personalized model 1940 and the calibration model 1950 have been properly trained they can be deployed and utilized by a particular user or patient. Once deployed, input data for the particular user can be received continuously. In this embodiment, this input data can include, but is not limited to, discrete glucose measurement data 1910 from the blood glucose meter for that particular user or patient, contextual data 1920 from the source of user activity data for that particular user or patient, and contextual data 1930 from the other sources for that particular user or patient. The personalized model 1940 can receive and process the input data to generate a continuous time-series of estimated glucose values 1970. The estimated glucose values 1970 can then be used for a variety of purposes. For instance, as one non-limiting example, the estimated glucose values 1970 can be used in conjunction with an insulin infusion system to provide CGM-like therapy to the particular user or patient without the need for glucose sensor. The calibration model 1950 can be used to calibrate personalized model 1940 to help improve the performance of the model and ensure that it is performing accurately.

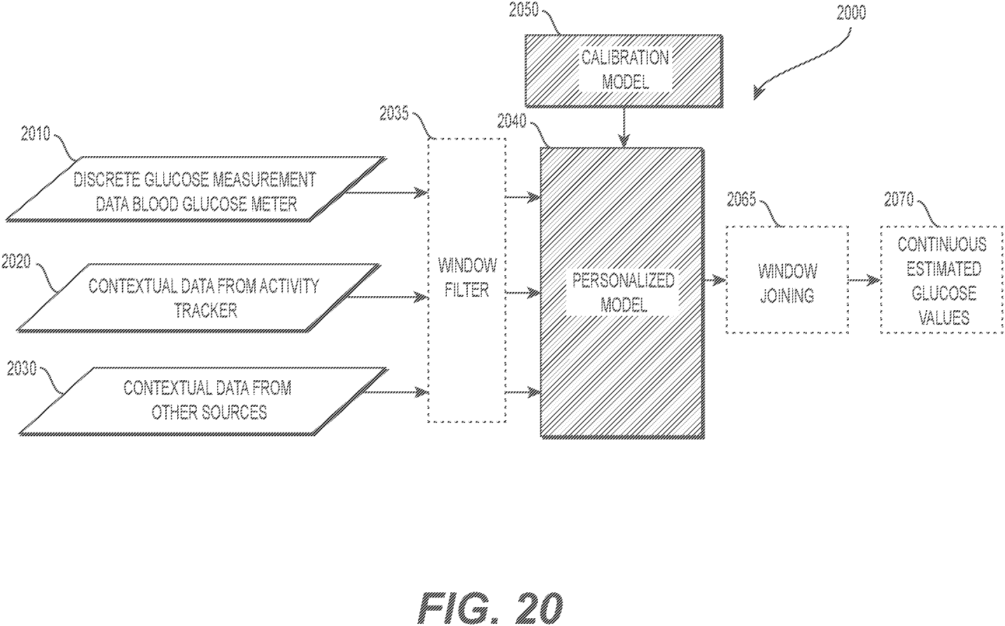

FIG. 20 is a block diagram that illustrates another intermittent CGM system 2000 in accordance with the disclosed embodiments. The structure of the intermittent CGM system 2000 is similar to the structure of the intermittent CGM system 1900 described above with reference to FIG. 19. As such, components or blocks shown in FIG. 19 will not be described in detail again in conjunction with FIG. 20 unless they perform differently in the embodiment of FIG. 19. In this embodiment, prior to providing the input data to the personalized model 2040 for processing, the data is provided to a window filter 2035 that can split the input data into different time windows. Each time window can include a discrete segment of the discrete glucose measurement data 2010 for that particular user or patient, contextual data 2020 from the source of user activity data for that particular user or patient, and contextual data 2030 for that particular user or patient. Data from each time window can then be processed at the personalized model 2040 to generate a time-series of estimated glucose values 2070 corresponding to a particular time window. In some embodiments, a window joining processor 2065 is provided that can join sets of the estimated glucose values 2070 corresponding to any number of time windows to generate a joined set of estimated glucose values 2070 over any number of time windows. For example, in some embodiments, estimated blood glucose sequences over each time window can be stored, and then be sequentially linked together (e.g., assembled/concatenated) at a later time. In some embodiments, the window joining step takes output of the previous window’s estimated glucose response to initialize the subsequent window. This may be done by replacing or modifying one of the inputs to contain a segment of, or the entire, estimated glucose response output personalized learning step applied to the previous window.

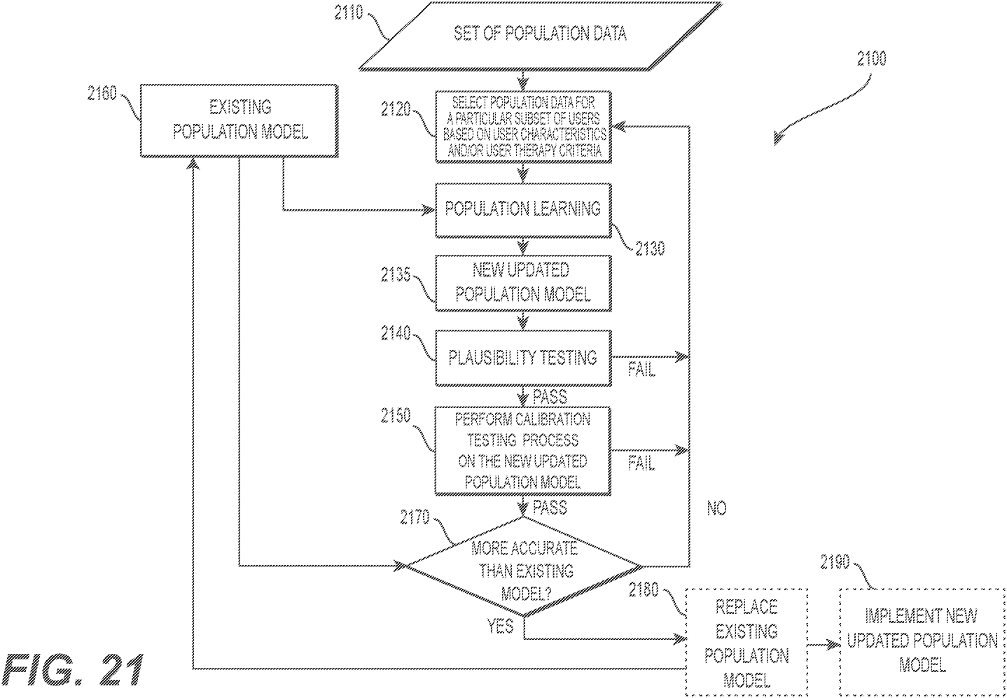

FIGS. 21-23 are flow charts that illustrate examples of model updating methods in accordance with the disclosed embodiments that can be used to iteratively update any of the models described herein. With respect to FIGS. 21-23, the steps of each method shown are not necessarily limiting. Steps can be added, omitted, and/or performed simultaneously without departing from the scope of the appended claims. Each method may include any number of additional or alternative tasks, and the tasks shown need not be performed in the illustrated order. Each method may be incorporated into a more comprehensive procedure or process having additional functionality not described in detail herein. Moreover, one or more of the tasks shown could potentially be omitted from an embodiment of each method as long as the intended overall functionality remains intact. Further, each method is computer-implemented in that various tasks or steps that are performed in connection with each method may be performed by software, hardware, firmware, or any combination thereof. For illustrative purposes, the following description of each method may refer to elements described herein. In certain embodiments, some or all steps of this process, and/or substantially equivalent steps, are performed by execution of processor-readable instructions stored or included on a processor-readable medium. For instance, in the description of FIGS. 21-23 that follows, it should be appreciated that steps of the methods refer to processor(s) or processing system(s) executing instructions to perform those various acts, tasks or steps. Depending on the implementation, some of the processor(s) or processing system(s) can be centrally located, or distributed among a number of systems that work together.

As described above, a population model can be used to directly predict or estimate blood glucose values, or to initialize the training of a personalized model for each particular user. Once a population model is deployed, there may be a need to update it if new, more appropriate, or better data becomes available for a larger or different population. To address these issues, an assessment may be performed to evaluate if an updated population model is similar or better than the existing population model that has been deployed.

FIG. 21 is a flowchart that illustrates a method 2100 for updating an existing population model 2160 for estimating glucose values for a population of particular users to generate a new updated population model 2135 for a subset of users of the population of users in accordance with the disclosed embodiments. As will be described in greater detail below, the existing population model 2160 can be regularly updated using the updated set of population data 2110 to create one or more new updated population model(s) 2135 for particular population subset(s). Each particular population subset can be a particular group or subset of users selected from the population of users (e.g., a particular group or subset of the population of users that share common user characteristics and/or therapy criteria). The number of users in the particular group or subset can vary, for example, depending on the common user characteristics and/or therapy criteria that are specified or selected, and in one implementation, the particular group or subset could include all of the users of the population of users (e.g., the common user characteristics and/or therapy criteria are specified or selected such that the subset is equal to the entire population). Ideally, the new updated population model 2135 that is generated or derived during each iteration of the method 2100 will be better tuned to improve performance in comparison to the existing population model 2160 for particular population subset(s). For example, the new updated population model 2135 that is generated or derived during each iteration of the method 2100 will ideally improve performance of the existing population model 2160 to provide improved estimates of glucose values for a particular subset of the population of particular users.

Inputs used by the method 2100 can include a set of population data 2110 for a population of users, and an existing population model 2160. A few non-limiting examples of sources of the population data 2110 can include, for example, one or more of: data from a glucose monitoring device associated with each particular user (e.g., therapy-related data and settings); data from a meal tracking system regarding consumption of macronutrients by each particular user from any type of device described herein (e.g., meal ingestion data regarding type of food and amount of food consumed by a particular user); contextual activity data from an activity tracker or gesture detection system associated with each particular user (e.g., activity-related data such as data regarding exercise routine of each particular user; data regarding sleep patterns of each particular user, etc.). Examples of sources of population data may be of the type described in, but not limited to, U.S. Pat. Application Serial Nos.: 17/120,052; 17/118,519; 17/120,054; 17/120,057; 17/120,001; and 17/120,055, each of which are hereby incorporated by reference in their entirety, except for any disclaimers, disavowals, and inconsistencies. To the extent that the incorporated material is inconsistent with the express disclosure herein, the language in this disclosure controls, and any inconsistent or conflicting information in the incorporated material is not incorporated by reference herein.

In some embodiments, the set of population data 2110 can be obtained, for example, from research studies, clinical studies, or real-world data collection, including a wide range of heterogenous subjects such as, diagnoses (e.g., Type 1 diabetes, Type 2 diabetes, pre-diabetes, etc.), different ages, insulin and carbohydrate sensitivities (e.g., insulin sensitivity factor (ISF), insulin carbohydrate ratio (ICR), etc.), body size and proportion, ethnicity, sex, etc.

The existing population model 2160 can be one that is initially provided at the start of the method 2100 (e.g., during a first iteration of method 2100) or one that has been updated during a previous iteration of the method 2100. In some embodiments, the existing population model 2160 that is initially provided at the start of the method 2100 can be generated, for example, using data for an initial population of users as described above with reference to FIGS. 4-13. The set of population data 2110 can be updated over time as new data is obtained (e.g., additional data for the initial population of users, or data for new users who are added to the set). As the set of population data 2110 is updated, this presents an opportunity to further refine or fine-tune the existing population model 2160 by either updating it based on the updated set of population data 2110 and the newly acquired data in that updated set of population data 2110, or alternatively creating an entirely new population model based on the updated set of population data 2110 and then using that as the existing population model 2160.

Model performance in real world evaluation depends on the patients included in the population model training step. Model parameters learned with patients substantially different from the test population can lead to model bias, and subsequently poor performance. It is critical to select an appropriate set a subset of patients from the population with characteristics similar to the type of patients the model will be applied to. For example, a model or algorithm applied to type 1 diabetes may benefit from a limited the training subset (e.g., only type 1 diabetes). In some instances, a more refined subset of patients considering multiple characteristics is needed to achieve satisfactory model performance. For example, training a model using data from patients with type 1 diabetes, who are also 18-30 years old, who also weigh 80-150 lbs., may improve performance for patients with similar characteristics, compared to a model trained with type 1 diabetes patients without any other restrictions. In other instances, multiple population models may be employed, instead of a single population model, in order to improve overall system performance.

Method 2100 starts at 2120, where population data for a particular population subset (e.g., group) of users is selected from the set of population data 2110 for a population of users. As will be explained below, depending on the implementation of method 2100, step 2120 can either select a new subset of users during each iteration of the method 2100, or alternatively, keep the subset of users that is selected constant during each iteration of the method 2100 (e.g., until a “yes” decision is reached at 2170).

In some embodiments, the subset of users that is selected (at 2120) can be those sharing, for example, common user characteristics and/or common therapy criteria. For instance, in some scenarios, the “selected population data,” that is selected from the set of population data, can be specified by defining a subset of users that share common user characteristics. In other scenarios, the “selected population data,” that is selected from the set of population data, can be specified by defining a subset of users that share common therapy criteria. In other scenarios, the “selected population data,” that is selected from the set of population data, can be specified by defining a subset of users that share common user characteristics and common therapy criteria. In still other embodiments, the subset of users can be randomly selected/specified. Any of these approaches can be applied to select the population data for the particular population subset (e.g., group) from the set of population data 2110. In some cases, when the goal is to create a new population model for a specific population of users, the subset may include only a portion of the set of population data 2110. This allows population models to be created for certain distinct populations. In other cases, when the goal is to refine or fine-tune the existing population model 2160 to improve the existing population model 2160 based on new data that has been acquired since the existing population model 2160 was created, the subset may include newly acquired data from the set of population data 2110. This allows the existing population model 2160 to be fine-tuned based on the newly acquired data from the set of population data 2110.

The set of population data 2110 can be, for example, a dataset sourced from a large population of multiple users that is available for training the existing population model 2160. The particular subset can be a group of users in that population that are selected based on any number of common user characteristics and/or any number of common therapy criteria, as will be describe below in greater detail. As will be explained below, in some implementations, each time the method 2100 is executed or iterated, different values (or ranges of values) for the user characteristics and/or therapy criteria can be defined or selected (at 2120) so that population learning 2130 (or training of the existing population model 2160) can be customized for a different population subset (e.g., group of users) that is selected from set of population data 2110 with the end goal being to create improved population models for use with different population subsets.

Additionally, in other implementations, the population subset can remain the same as was selected during an initial iteration of method 2100, and each time the method 2100 is executed or iterated, the model (or combination of models) used at 2130 can be changed or varied so that population learning 2130 (or training of the existing population model 2160) can be customized for the same population subset (e.g., group of users) that was selected (at 2120) from set of population data 2110 with the end goal also being to create improved population models for use with that same population subset. As such, in these implementations, a new or different population subset is not selected (at 2120) during each iteration of the method 2100. Rather, a single population subset can be selected at 2120 during the first iteration of method 2100 and kept constant during subsequent iterations of the method 2100 (e.g., until a “yes” determination is reached at 2170). In this case, the population model can be updated at 2130, for example, by utilizing different modeling techniques during each iteration to generate different updated population models, which can then be evaluated at 2140, 2150 and 2170. Thus, each time population learning is executed at 2130, a new model (or multiple models) can be generated that has different architecture, different parameters, or that utilizes different learning techniques. In the end, the model with the highest accuracy, which is the one that meets all criteria 2140 through 2170, and is thus more accurate than the existing population model, can be selected.

Depending on the implementation, any number of different user characteristics can be selected to define a group (e.g., specific subset) of users that are to be included as part of the particular population subset. Depending on the implementation, the user characteristics can be selected by a manual process or automated process (e.g., randomly selected or selected via an algorithmic process). A few non-limiting examples of the different user characteristics that can be used to select the subset of the population of particular users can include, for example: an age value (or age range) for the subset of the population of particular users, sex of the subset of the population of particular users, height (or range of heights) for the subset of the population of particular users, weight (or weight range) of the subset of the population of particular users, an activity level of the subset of the population of particular users, a type of diabetes of the subset of the population of particular users (e.g., type 1 population vs. type 2 population), an amount and/or timing of macronutrients typically consumed by the subset of the population of particular users, etc.

Depending on the implementation, values or ranges for any number of different therapy criteria can also be selected by a manual or automated process (e.g., randomly selected or selected via an algorithmic process) to further define the population data that will be include as part of the particular population subset. A few non-limiting examples of the different therapy criteria that can be selected for the subset of the population of particular users can include, for example, therapy parameters for the subset of the population of particular users, such as: a basal profile of the subset of the population of particular users, an active insulin time (or range) of the subset of the population of particular users, an insulin sensitivity factor (or range) for the subset of the population of particular users, an insulin-to-carbohydrate ratio (or range) for the subset of the population of particular users, a total daily dose of insulin delivered (or range) of the subset of the population of particular users, a bolus pattern or schedule of the subset of the population of particular users, a type of insulin used by the subset of the population of particular users, etc.

Additional examples of different user characteristics and different therapy criteria that can be used to define a subset of the population data for a group of particular users may be described, for example, in U.S. Pat. Application Serial Nos.: 17/120,052; 17/118,519; 17/120,054; 17/120,057; 17/120,001; and 17/120,055.

Referring again to FIG. 21, at 2130, the existing population model 2160 can be trained based on the selected subset of the population data to perform population learning and generate the new updated population model 2135. The new updated population model 2135 simulates a physiological blood glucose response (i.e., estimated glucose response output) in response to various inputs. In some embodiments, the population learning techniques that are described above with respect to FIGS. 5-13 can be implemented at 2130 to perform population learning to generate the new updated population model 2135. Population learning (at 2130) may vary depending on the implementation. For instance, in some implementations, population learning (at 2130) may involve transfer learning (e.g., starting from existing population model and refining it by updating its parameters). In some implementations, population learning (at 2130) may involve combining the existing population model output with a new model. In some implementations, population learning (at 2130) may involve a variety of machine learning techniques to develop a brand new model that is independent of the existing model.

The accuracy of the estimated glucose response output of the new updated population model 2135 can vary depending on the model. In some cases, the accuracy of the estimated glucose response output of the new updated population model 2135 will be physiologically appropriate given in the inputs, whereas in other cases, the accuracy of the estimated glucose response output of the new updated population model 2135 will not be physiologically appropriate given in the inputs. For example, a typical human physiological response to food intake is a rise or increase in blood glucose levels (e.g., glucose is expected to increase with increased carbohydrate consumption). By contrast, a typical human physiological response to an increased insulin intake is a drop or decrease in blood glucose levels. Ideally, the estimated glucose response from the new updated population model 2135 should react in a physiologically accurate manner under changes to inputs (e.g., carbohydrate, insulin, exercise, sleep, etc.).

At 2140, a plausibility testing process can be performed to determine whether an estimated glucose response output of the new updated population model 2135 changes in a physiologically appropriate manner in response to predetermined inputs when processed by the new updated population model 2135. The estimated glucose response can include estimates of glucose values for the subset of users. For example, in some embodiments of 2140, the new updated population model 2135 may be tested to ensure that the estimated glucose response it produces is at least directionally similar and/or similar in magnitude to what is expected based on clinical, medical, or physiological observations in response to the other inputs. In some embodiments, the model explainability analysis techniques that are described above with respect to FIGS. 10 and 11 can be implemented to perform the plausibility testing process at 2140.

For example, in some embodiments, each of the inputs (e.g., insulin and/or carbohydrates) to the new updated population model 2135 may be systematically modified (e.g., increased or decreased), and passed through the new updated population model 2135. This systematic modification can involve modifying (or modulating) the inputs into the new updated population model 2135 to generate the estimated glucose response to that set of inputs. For instance, the magnitude of each of the inputs into the model can be scaled, and/or timing of each of the inputs into the model can be time-shifted.

For each modification of the inputs, the effect on the estimated glucose response of the new updated population model 2135 can then be observed (over any number of different time scales) and evaluated by comparing the estimated glucose response of the new updated population model 2135 to an expected glucose response that has been established for those inputs. For example, the estimated glucose response of the new updated population model 2135 can be evaluated by comparing the direction (e.g., increase or decrease) of the estimated glucose response to the expected glucose response that has been established for those inputs. Additionally, or alternatively, the estimated glucose response of the new updated population model 2135 can be evaluated by comparing the magnitude of the estimated glucose response to the magnitude of expected glucose response that has been established for those inputs (e.g., compare the estimated glucose response against expected level of increase or decrease).

To determine whether the estimated glucose response output of the new updated population model 2135 changes in a physiologically appropriate manner, the plausibility testing process 2140 can determine whether the estimated glucose response output of the new updated population model 2135 to a set of predetermined inputs is within an appropriate error threshold (or degree of error) with respect to an expected glucose response in response to that same set of predetermined inputs (e.g., a reference or standard that has been clinically, medically, or physiologically determined to be “physiologically appropriate” in response to a given set of inputs).

When the estimated glucose response output of the new updated population model 2135 to the predetermined inputs is determined not to be within an appropriate error threshold (e.g., degree of error) with respect to the expected glucose response to those same set of inputs (at 2140), the new updated population model is not sufficiently plausible (e.g., the new updated population model 2135 fails the plausibility testing process), and the new updated population model can be removed from further consideration (e.g., disregarded or discarded). The method 2100 loops to 2120, and steps 2120-2170 can be repeated.

By contrast, when the estimated glucose response output of the new updated population model to the predetermined inputs is determined to be within an appropriate error threshold (e.g., degree of error) with respect to the expected glucose response to the same set of predetermined inputs (at 2140), the new updated population model 2135 is sufficiently plausible (e.g., the new updated population model 2135 passes the plausibility testing process). In some implementations, the new updated population model 2135 can then be calibrated via a calibration point testing process (at 2150). To explain further, in some examples, prior to evaluating performance of the new updated population model 2135 at 2170, it may be desirable to calibrate the new updated population model 2135, for example, using a blood glucose value from a measurement device, or alternatively, by using an estimated blood glucose value(s) derived from statistical analysis of historical patient data. As such, in some embodiments, the new updated population model 2135 can be calibrated via the calibration point testing process (at 2150) prior to proceeding to 2170. By contrast, in other examples, the method 2100 may proceed directly to 2170 without performing the calibration point testing process (at 2150).

At 2150, a calibration point testing process can be performed on the new updated population model 2135 to evaluate performance of the new updated population model 2135.

In some examples, the calibration point testing process that is performed at 2150 can include evaluating the performance of the new updated population model 2135 at different calibration intervals to determine which calibration interval is optimal for the new updated population model 2135.

In other examples, the calibration point testing process that is performed at 2150 can also include developing new calibration models, then evaluating the new calibration models to determine which one is to be used to evaluate the performance of the new updated population model 2135, and then using that particular one of the newly-developed calibration models to evaluate the performance of the new updated population model 2135 (e.g., at different calibration intervals to determine which calibration interval is optimal for the new updated population model 2135). In still other examples, the calibration point testing process that is performed at 2150 can also include developing new calibration models, then evaluating the new calibration models to determine which ones are to be used to evaluate the performance of the new updated population model 2135, and then using each of those particular ones of the newly-developed calibration models to evaluate the performance of the new updated population model 2135 (e.g., at different calibration intervals for each of the new updated population model 2135 to determine which calibration interval is optimal for the new updated population model 2135), and then determining which optimal calibration interval for one of the one of newly-developed calibration models is to be used for calibrating the new updated population model 2135. In some embodiments, the calibration point testing process that is performed at 2150 can include the processing that is described above with respect to FIGS. 8-10 at 866, 966, 1066, respectively, and FIGS. 12-13. Thus, depending on the implementation, the calibration point testing process may, for example, evaluate pre-specified calibration intervals, or test various calibrations models and strategies to reduce the calibration interval on the new updated population model 2135.

In some embodiments, the calibration point testing process that is performed at 2150 can test performance of the new updated population model 2135 by changing the calibration interval (e.g., calibrate one time per day, one time per week, one time per month, or any other pre-determined period of time) and evaluating whether the new updated population model 2135 satisfies performance criteria at each calibration interval (e.g., determine whether the new updated population model 2135 performs satisfactorily at the desired calibration interval or calibration rate). The calibration interval defines the frequency or how often the new updated population model 2135 need to be calibrated using one or more blood glucose value(s) (e.g., obtained from a blood glucose measurement device such as a blood glucose meter) as an input to the new updated population model 2135. The calibration interval can be specified or defined as a number of time units (e.g., a number of minutes, hours, weeks, months, etc.) that define how often the new updated population model 2135 needs to be calibrated using one or more blood glucose values as an input to the new updated population model 2135. As such, the calibration interval determines the number of times the new updated population model 2135 needs to be calibrated in a given period of time (e.g., the number of calibrations per day, week, month, etc.).

For example, at each calibration interval that is tested, a calibration optimization processor (not shown in FIG. 21) can determine whether the new updated population model 2135 satisfies performance criteria when it is calibrated at that calibration interval. For instance, at each calibration interval that is tested, the calibration optimization processor can determine a performance score for the new updated population model 2135 when it is calibrated at that calibration interval, and determine whether that performance score is greater than or equal an error threshold. Each performance score can reflect or is indicative of the accuracy of glucose estimates produced by the new updated population model 2135 when it is calibrated at that particular calibration interval using a particular calibration model. The performance score can be determined by evaluating various performance metrics or criteria that can vary depending on the implementation (e.g., root mean squared error, mean relative difference, detectability of glucose excursions, accuracy based on time of day, etc.).

Calibration intervals that are determined to have performance scores that are greater than or equal the error threshold can be further evaluated. In some embodiments, the one of those calibration intervals that has the longest duration (i.e., the longest calibration interval) can be selected as the “optimized” calibration interval to be used in conjunction with the new updated population model 2135. The optimized calibration interval will have a performance score that indicates that glucose estimates produced by the new updated population model 2135 have a sufficient level accuracy when it is calibrated at that particular calibration interval, while also having the longest duration. Stated differently, the optimized calibration interval can indicate how often blood glucose value(s) need to be provided as input to that new updated population model 2135 to achieve an acceptable level of accuracy in estimating glucose values for the population of users. In general, the longer the interval the better because it is undesirable to calibrate frequently because fewer calibrations over a given time period are needed, which is less of a burden to the user (e.g., lower number of finger-sticks per unit time are required to calibrate the population model).

As such, the calibration testing process can be used to evaluate different calibration intervals and/or different calibration models (e.g., processes or approaches) to determine which calibration interval provides the optimal calibration interval for that new updated population model 2135. This way, the calibration testing process can be used to optimally determine how often measured blood glucose value(s) need to be provided as input to that new updated population model 2135 to achieve a certain acceptable level of performance. In some embodiments, the calibration optimization processor (not shown in FIG. 21) can optimize calibration intervals and/or calibration error (e.g., longer intervals between calibration and/or reduced calibration error). For example, the calibration optimization processor can optimize calibration characteristics by determining which calibration interval will provide the best compromise between low calibration error and long calibration interval. For instance, in some implementations, a required minimum calibration interval may be specified that is less than or equal to a specified duration. The calibration interval (and accompanying calibration approach) that is less than or equal to the required minimum calibration interval while yielding the highest performance can be selected for the new updated population model.

If the performance of the new updated population model 2135 is better than the existing population model 2160, using the same calibration interval, then the new updated population model 2135 is considered better. If the new updated population model 2135 has a longer calibration interval and has better performance than the existing population model 2160, then both the new updated population model 2135 and the calibration interval may be adopted. In the event none of the calibration intervals result in a performance score that is above error threshold, the new updated population model 2135 will be deemed to have failed the calibration testing process, and the new updated population model 2135 will be disregarded from further consideration. For instance, if the new updated population model 2135 has to be calibrated too frequently (e.g., 5x per day) to perform well, this may be too frequent (e.g., too much burden to the user), and that new updated population model 2135 will not be considered further. Thus, if none of the calibration intervals can achieve a sufficient level of accuracy, the new updated population model 2135 can be removed from consideration or rejected.

At 2170, performance of the existing population model 2160 can be compared to performance of the new updated population model 2135 to determine which model provides better estimates of glucose values for the population of particular users. In one implementation, a predetermined testing dataset can be applied to both the new updated population model 2135 and the existing population model 2160, and an estimated glucose response of the existing population model 2150 to the predetermined testing dataset can then be compared to the estimated glucose response of the new updated population model 2135 to the predetermined testing dataset to determine which model provides more accurate estimates of glucose values for the subset of users. In another implementation, a different subset or portion of the set of population data 2110 (e.g., data that was not used to develop the new updated population model 2135) can be applied to both the new updated population model 2135 and the existing population model 2160 as a predetermined testing dataset, and an estimated glucose response of the existing population model 2150 to the predetermined testing dataset can then be compared to the estimated glucose response of the new updated population model 2135 to the predetermined testing dataset to determine which model provides more accurate estimates of glucose values for the subset of users.

When it is determined (at 2170) that the performance of the new updated population model 2135 does not provide more accurate estimates of glucose values for the subset of users (that was selected at 2120), the new updated population model 2135 is removed from consideration (e.g., rejected, ignored, discarded, disregarded), and the method 2100 can loop back to 2120, where steps 2120-2170 can be repeated for a different subset of the population 2110 of particular users. For example, in some embodiments, if performance scores of the new updated population model 2135 are lower than performance scores of the existing population model 2150, it can be removed from further consideration.

By contrast, when it is determined (at 2170) that the performance of the new updated population model 2135 provides more accurate estimates of glucose values for the subset of users (that was selected at 2120), then the existing population model 2160 can be updated (at 2180) with the new updated population model 2135, and at 2190, the new updated population model 2135 can be implemented or deployed for usage with the subset of users. As such, if performance of the existing population model is improved (e.g., when the estimated glucose response of the new updated population model 2135 provides more accurate estimates of glucose values for the subset of users than the existing population model 160), the existing population model 2160 can be replaced with the new updated population model 2135. The method 2100 can be regularly repeated to iteratively update a most recently updated version of population model.

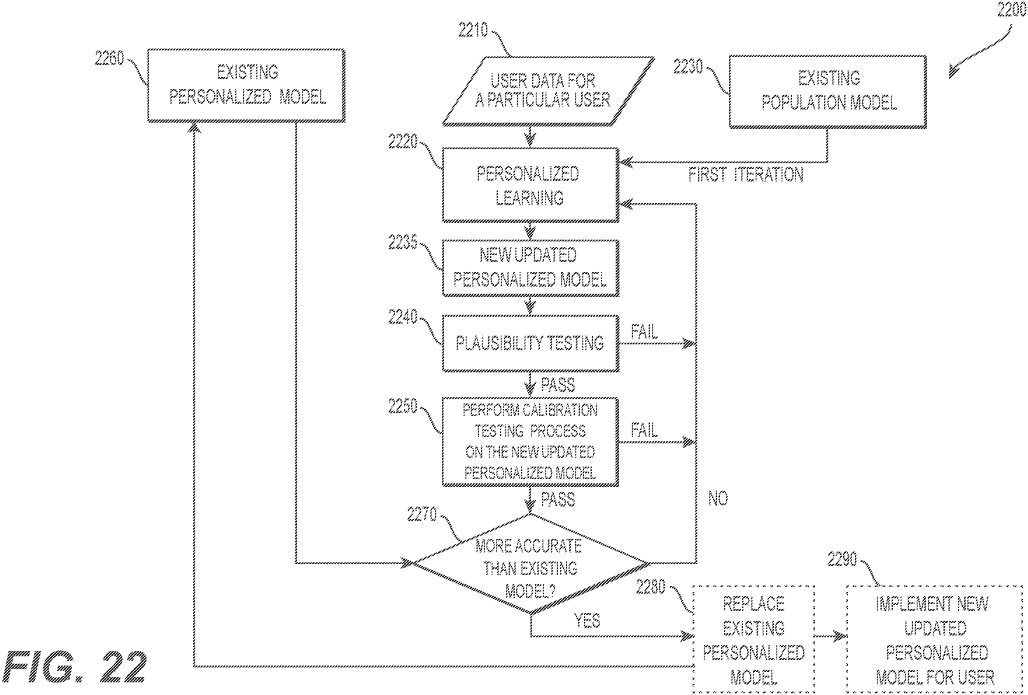

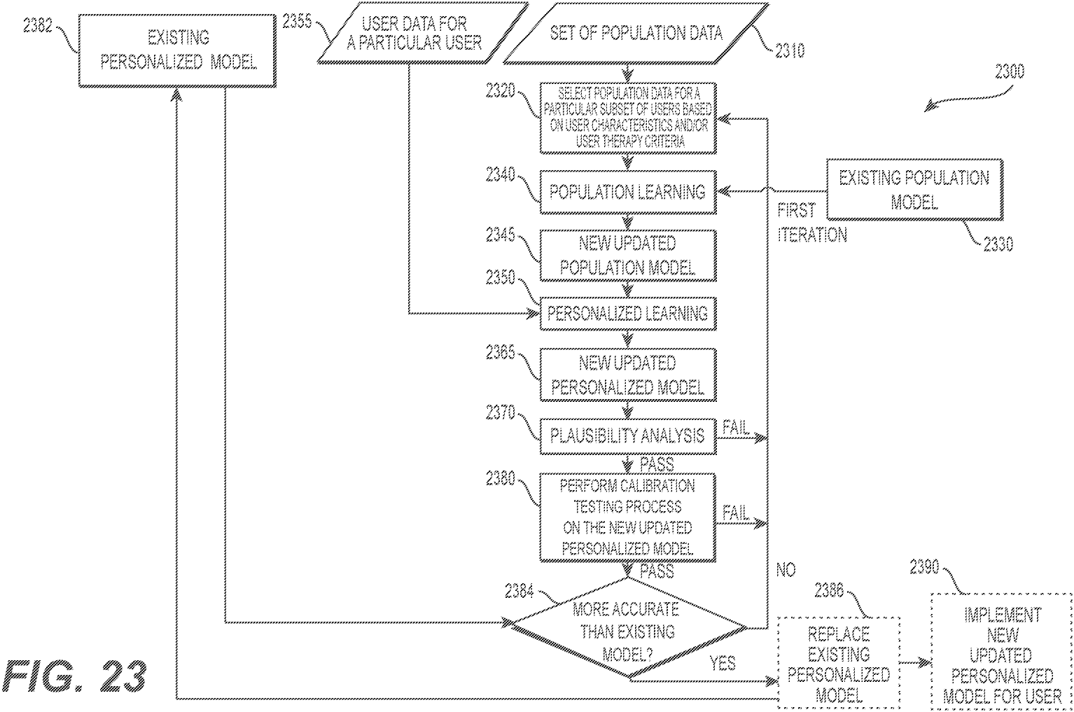

As described above, any population model (including those described above) may be selected based on an individual user’s characteristics, and instantiated to initialize personalized training of a personalized model for a particular individual, patient or user. The personalized model can provide a more precise representation of an individual user’s physiology. Once an existing personalized model is deployed to a user device or service, there may be a need to re-train it as new user specific data becomes available. Re-training and updating the personalized model can help mitigate performance drift and to adapt to changes in a patient’s behavior (e.g., exercise routine, sleep patterns, food consumption including amounts of carbohydrates, insulin dosing information, etc.). To address these issues, methodologies are provided for updating (e.g., retraining) an existing personalized model as when new, user-specific data, is acquired and becomes available. This way, the personalized model can better learn the factors impacting glucose as they change over time (e.g., physiological changes in insulin sensitivity, varying food consumption behaviors of a user, etc.) This can allow for the personalized model to be better personalized to account for and to adapt to changes in the physiology and/or behavior of the particular user as they change over time. Examples of these methodologies will now be described with respect to method 2200 of FIG. 22 and method 2300 of FIG. 23.

FIG. 22 is a flowchart that illustrates a method 2200 for updating an existing personalized model 2260 for estimating glucose values of a particular user in accordance with the disclosed embodiments. Method 2200 can be applied to the existing personalized model 2260 used to generate a new updated personalized model 2235 that is personalized for a particular user. The new updated population model 2235 that results can improve performance of the existing personalized model 2260 by providing improved or more accurate estimates of glucose values for that particular user.

The method 2200 starts at 2220, where a personalized learning process is performed. On a first iteration of method 2200, an existing population model 2230 can be used to initialize parameters of an existing personalized model 2260 that is to be adapted based on user data 2210 for a particular user. For example, after the existing personalized model 2260 is initialized, the existing personalized model 2260 is trained based on a subset of user data 2210 for the particular user (e.g., data that reflects physiology of the particular user) to generate or derive the new updated personalized model 2235.

At least some of the user data 2210 for the particular user can be used as training data for updating the existing personalized model 2260. The user data 2210 can be updated on a regular basis and thus changes over time as new user data is acquired. For example, new user data can be added to the user data 2210 as it is acquired, and/or removed from the user data 2210 over time (e.g., as the data ages). While all of the user data 2210 could be used as training data for updating the existing personalized model 2260, in many cases, it is desirable to select a subset of user data 2210 as the training data for updating the existing personalized model 2260. In some cases, a new subset of user data can be selected during each iteration of the method 2200. Each new subset of user data may include at least some new data that is acquired since the previous iteration of method 2200. As such, during each iteration of the method 2200, the new subset of user data may include new user data that was acquired after a previous iteration of the method 2200, which means that each new subset of user data 2210 that is selected as training data for updating the most recent version of the existing personalized model 2260 can include at least some new data that is acquired after that most recent version of the existing personalized model 2260 was generated.

In some embodiments, the user data 2210 for the particular user may be split into one or more datasets that can be used for different purposes, such as training, validation (e.g., model tuning) and performance testing datasets. For instance, as one non-limiting example, user data for the particular user from days 1-7 may be selected for a training dataset, while user data for the particular user from day 8 may be selected for a validation dataset, while user data for the particular user from days 9-14 may be selected for a performance testing dataset. Similarly, as another non-limiting example, user data for the particular user from day 1 may be selected for a validation dataset, user data for the particular user from days 2-8 may be selected for a training dataset, and user data for the particular user from days 9-14 may be selected for a performance testing dataset. The training and validation data may be interlaced, interleaved or striated, such that a portion of day 1-8 is used for a training dataset and another portion is used for a validation dataset. The number of weeks/days/hours/minutes used for the training dataset, the validation dataset and the testing dataset is flexible and dependent on the use case and business needs.

In some embodiments, the user data can include historical data, such as, data from a glucose monitoring device associated with the particular user (e.g., discrete glucose measurement data from the blood glucose meter or other sensor arrangement that provides discrete glucose measurements, etc.); data regarding consumption of macronutrients by the particular user (e.g., data from the other sources such as nutritional information about meals consumed by a user, insulin delivered to the user by an insulin infusion device of the user, etc.); activity data associated with the particular user including data regarding exercise routine of the particular user; data regarding sleep patterns of the particular user; and any other contextual data collected from a device associated with the particular user (e.g., activity data from an activity tracker, electrodermal activity sensor, temperature sensor, oxygen monitor, etc.).

At 2240, a plausibility testing process is performed to determine whether an estimated glucose response that is output by the new updated personalized model 2235 changes in a physiologically appropriate manner in response to modified user data for the particular user, that is different than the new user data 2210, when it is processed by the new updated personalized model 2235. The estimated glucose response can include estimates of glucose values for the particular user. Similar to the new user data 2210, the modified user data can be, for example, data from a glucose monitoring device associated with the particular user, data regarding consumption of macronutrients by the particular user, and/or contextual activity data associated with the particular user. The plausibility testing process at 2240 can vary depending on the implementation, and in some examples, can be implemented in a manner similar to that as described above with reference to step 2140 of FIG. 21 except that it is applied to the new updated personalized model 2235 at 2240 (as opposed to the new updated population model 2135 in FIG. 21). At step 2240, the new user data 2210 is modified to assess the impact of various inputs (e.g., carbohydrates, insulin, exercise, etc.) on the estimated glucose response that is output by the new updated personalized model 2235. For example, a carbohydrate input may be modified to generate a modified set of inputs, which are run through the new updated personalized model 2235 to generate an estimated glucose response. Similarly, an insulin bolus input may be modified to generate a modified set of inputs. Each modified set of inputs can be run through the new updated personalized model 2235, and the estimated glucose response that is output by the new updated personalized model 2235 can be evaluated for plausibility.

For example, in some embodiments of 2240, the new updated personalized model 2235 may be tested to ensure that the estimated glucose response it produces is at least directionally similar and/or similar in magnitude to what is expected based on clinical, medical, or physiological observations in response to the other inputs. In some examples, the model explainability analysis techniques described herein can be implemented to perform the plausibility testing process at 2240.

In some examples, each of the inputs (e.g., insulin and/or carbohydrates) to the new updated personalized model 2235 may be systematically modified (e.g., increased or decreased), passed through the new updated personalized model 2235, and the estimated glucose response output of the new updated personalized model 2235 can be verified for an expected glucose response. This systematic modification can involve modifying (or modulating) the inputs into the new updated personalized model 2235 to generate the estimated glucose response to that set of inputs. For instance, the magnitude of each of the inputs into the model can be scaled, and/or timing of each of the inputs into the model can be time-shifted.

For each modification of the inputs, the effect on the estimated glucose response of the new updated personalized model 2235 can then be observed (over any number of different time scales) and evaluated by comparing the estimated glucose response of the new updated personalized model 2235 to an expected glucose response that has been established for those inputs. Criteria (or conditions) for passing or failing this plausibility testing can be established based on the degree of error in reactivity to the different inputs. For example, in some examples, the plausibility testing criteria may be set, for example, on the direction (e.g., increase, decrease) and/or magnitude of the estimated glucose response output to different types of inputs to the new updated personalized model 2235. For instance, the estimated glucose response of the new updated personalized model 2235 can be evaluated by comparing the direction (e.g., increase or decrease) of the estimated glucose response to the expected glucose response that has been established for those inputs. Additionally, or alternatively, the estimated glucose response of the new updated personalized model 2235 can be evaluated by comparing the magnitude of the estimated glucose response to the magnitude of expected glucose response that has been established for those inputs (e.g., compare the estimated glucose response against expected level of increase or decrease).

To determine whether the estimated glucose response output of the new updated personalized model 2235 changes in a physiologically appropriate manner, the plausibility testing process 2240 can determine whether the estimated glucose response output of the new updated personalized model 2235 (to a set of modified user data) is within an appropriate error threshold (or degree of error) with respect to an expected glucose response in response to that same set of modified user data (e.g., a reference or standard that has been clinically, medically, or physiologically determined to be “physiologically appropriate” in response to a given set of inputs).

When the plausibility testing process (at 2240) indicates that the new updated personalized model 2235 is not sufficiently plausible, the new updated personalized model can be discarded and the method 2200 loops to 2220, and steps 2220-2270 can be repeated. For example, in some examples, when the estimated glucose response output of the new updated personalized model 2235 to the modified user data is determined not to be within an appropriate error threshold (e.g., degree of error) with respect to the expected glucose response to those same set of inputs (at 2240), the new updated personalized model is not sufficiently plausible (e.g., the new updated personalized model 2235 fails the plausibility testing process), and the new updated personalized model can be removed from further consideration (e.g., ignored, disregarded or discarded). The method 2200 loops to 2220, and steps 2220-2270 can be repeated.

By contrast, when plausibility testing process of the new updated personalized model 2235 (at 2240) indicates that it is sufficiently plausible it can be evaluated further and the method 2200 proceeds to 2250 (or 2270 if 2250 is not performed). For example, in some examples, when the estimated glucose response output of the new updated personalized model to the modified user data is determined to be within an appropriate error threshold (e.g., degree of error) with respect to the expected glucose response to the same set of modified user data (at 2240), the new updated personalized model 2235 can be determined sufficiently plausible (e.g., the new updated personalized model 2235 passes the plausibility testing process).

In some examples, the new updated personalized model 2235 may or may not need to be calibrated using blood glucose value(s). As such, in some implementations, the new updated personalized model 2235 can then be calibrated via a calibration point testing process (at 2250). To explain further, in some examples, prior to evaluating performance of the new updated personalized model 2235 at 2270, it may be desirable to calibrate the new updated personalized model 2235, for example, using a blood glucose value from a measurement device, or alternatively, by using an estimated blood glucose value(s) derived from statistical analysis of historical patient data. As such, in some examples, the new updated personalized model 2235 can be calibrated via the calibration point testing process (at 2250) prior to proceeding to 2270. By contrast, in other examples, the method 2200 may proceed directly to 2270 without performing the calibration point testing process (at 2250).

As such, in some examples, a calibration point testing process can be performed on the new updated personalized model 2235 (at 2250) to evaluate performance of the new updated personalized model 2235 at different calibration intervals, and determine which calibration interval is optimal for the new updated personalized model 2235. In some examples, the calibration point testing process that is performed at 2250 can include the processing that is similar to that described above with respect to FIG. 21.

In some examples, the calibration point testing process that is performed at 2250 can test performance of the new updated personalized model 2235 by changing the calibration interval (e.g., calibrate one time per day, one time per week, one time per month, etc.) and evaluating whether the new updated personalized model 2235 satisfies performance criteria at each calibration interval (e.g., determine whether the new updated personalized model 2235 performs satisfactorily at the desired calibration interval or calibration rate). The calibration interval defines the frequency or how often the new updated personalized model 2235 need to be calibrated using one or more blood glucose value(s) (e.g., obtained from a blood glucose measurement device such as a blood glucose meter) as an input to the new updated personalized model 2235. The calibration interval can be defined as a number of time units (e.g., a number of minutes, hours, weeks, months, etc.). As such, the calibration interval determines the number of times the new updated personalized model 2235 needs to be calibrated in a given period of time (e.g., the number of calibrations per day, week, month, etc.).

For example, at each calibration interval that is tested, a calibration optimization processor (not shown in FIG. 22) can determine whether the new updated personalized model 2235 satisfies performance criteria when it is calibrated at that calibration interval. For instance, at each calibration interval that is tested, the calibration optimization processor can determine a performance score for the new updated personalized model 2235 at each calibration interval when it is calibrated at that calibration interval, and determine whether that performance score is greater than or equal an error threshold. Each performance score can reflect or is indicative of the accuracy of glucose estimates produced by the new updated personalized model 2235 when the new updated personalized model 2235 is calibrated at a particular calibration interval. Calibration intervals that are determined to have performance scores that are greater than or equal the error threshold can be further evaluated. In some examples, the one of those calibration intervals that has the longest duration (i.e., the longest calibration interval) can be selected as the “optimized” calibration interval to be used in conjunction with the new updated personalized model 2235. The optimized calibration interval will have a performance score that indicates that glucose estimates produced by the new updated personalized model 2235 have a sufficient level accuracy when it is calibrated at that particular calibration interval, while also having the longest duration. Stated differently, the optimized calibration interval can indicate how often blood glucose value(s) need to be provided as input to that new updated personalized model 2235 to achieve an acceptable level of accuracy in estimating glucose values for the particular user. In general, the longer the interval the better because it is undesirable to calibrate frequently because fewer calibrations over a given time period are needed, which is less of a burden to the user (e.g., lower number of finger-sticks per unit time are required to calibrate the population model).

As such, the calibration testing process can be used to evaluate different calibration intervals and determine which calibration interval provides the optimal calibration interval for that new updated personalized model 2235. This way, the calibration testing process can be used to optimally determine how often measured blood glucose value(s) need to be provided as input to that new updated personalized model 2235 to achieve a certain acceptable level of performance. In some examples, the calibration optimization processor (not shown in FIG. 21) can optimize calibration intervals and/or calibration error (e.g., longer intervals between calibration and/or reduced calibration error). For example, the calibration optimization processor can optimize calibration characteristics by determining which calibration interval will provide the best compromise between low calibration error and long calibration interval.

If the performance of the new updated personalized model 2235 is better than the existing personalized model 2260, using the same calibration interval, then the new updated personalized model 2235 is considered better. If the new updated personalized model 2235 has a longer calibration interval and has better performance than the existing personalized model 2260, then both the new updated personalized model 2235 having the calibration interval may be adopted. In the event none of the calibration intervals result in a performance score that is above error threshold, the new updated personalized model 2235 will be deemed to have failed the calibration testing process, and the new updated personalized model 2235 will be disregarded from further consideration. For instance, if the new updated personalized model 2235 has to be calibrated too frequently (e.g., 5x per day) to perform well, this may be too frequent (e.g., too much burden to the user), and that new updated personalized model 2235 will not be considered further. Thus, if none of the calibration intervals can achieve a sufficient level of accuracy, the new updated personalized model 2235 can be removed from consideration or rejected.

At 2270, the performance of the existing personalized model 2260 (e.g., that has already been deployed) can be compared to the performance of the new updated personalized model 2235 to determine which model provides more accurate (e.g., better) estimates of glucose values for the particular user. In some examples, to compare performance of the models 2235, 2260, a predetermined testing dataset (described above) can be applied to the new updated personalized model 2235 and to the existing personalized model 2260, and an estimated glucose response of the existing personalized model 2260 to the predetermined testing dataset can then be compared to the estimated glucose response of the new updated personalized model 2235 to the predetermined testing dataset to determine which model provides more accurate estimates of glucose values for the particular user.. In some examples, performance scores of the new updated personalized model 2235 can be compared to those for the existing personalized model 2260 to compare performance of the models 2235, 2260 and determine which one provides more accurate estimates of glucose values for the particular user.

In some implementations, the existing personalized model 2260 can have a specific calibration interval associated with it, and the new updated personalized model 2235 can include a set of calibration interval (and accompanying calibration approaches) along with the performance metrics for each. In such implementations, the existing personalized model 2260 having specified calibration interval (e.g., 7 days) can be compared with the new updated personalized model with same or longer calibration interval (e.g., 7-days or longer), and if the performance of the new updated personalized model 2235 (at any of the considered calibration intervals) is better than the existing personalized model 2260, the new updated personalized model 2235 may be selected .

When it is determined (at 2270) that the performance of the new updated personalized model 2235 does not provide more accurate estimates of glucose values for the particular user, the new updated personalized model 2235 is removed from consideration (e.g., rejected, ignored, discarded, disregarded), and the method 2200 can loop back to 2220, where steps 2220-2270 can be repeated. For example, if performance scores of the new updated personalized model 2235 are lower than performance scores of the existing personalized model 2250, it can be removed from further consideration.

By contrast, when it is determined (at 2270) that the performance of the new updated personalized model 2235 provides more accurate estimates of glucose values for the particular user, then the existing personalized model 2260 can be updated or replaced (at 2280) with the new updated personalized model 2235. At 2290, the new updated personalized model 2235 can be implemented or deployed for usage with the particular user. As such, if performance of the existing personalized model 2260 is improved (when the estimated glucose response of the new updated personalized model 2235 provides more accurate estimates of glucose values for the particular user than the existing personalized model 160), the existing personalized model 2260 can be replaced with the new updated personalized model 2235 and it can be deployed, for example, to a system or device associated with the particular user. As indicated by the feedback loops in FIG. 22, the method 2200 can be regularly repeated to iteratively update the most recently version of personalized model for that particular user (e.g., as new user data is added at 2210).

As described above, a population model can be used to initialize training of a personalized model. The population model that is utilized to initialize training can have significant influence on personalized model learning. Improving the quality of the population model may lead to improvements in performance of the personalized model. While the methodologies described with reference to FIGS. 21 and 22 can be implemented separately, they may also be implemented together as will now be described with reference to FIG. 23, where a methodology is provided that incorporates both population model learning in combination with personalized model learning, such that a series of population models can be evaluated for their impact on personalized model, and thus help to identify an optimal population model to be used to generate a personalized model.

FIG. 23 is a flowchart that illustrates a method 2300 for updating both an existing population model and an existing personalized model 2382 to generate a new updated personalized model that is personalized for a particular user (e.g., to adapt to changes in behavior of the particular user) in accordance with the disclosed examples. Method 2300 incorporates both population model learning (at 2340) in combination with personalized model learning (at 2350), such that a series of population models can be iteratively evaluated for their impact on personalized model, and thus help to identify an optimal population model to be used to generate or derive a personalized model. The new updated population model 2345 can improve performance of the existing personalized model 2382 by providing improved estimates of glucose values for that particular user.

The method 2300 of FIG. 23 differs from the method of FIG. 22 in that the method 2300 includes additional steps 2320 and 2340 that may be performed to train and update one or more population models that are then used when training a personalized model at 2350, instead of using the existing population model 2230 as described above with reference to FIG. 22. Additional steps 2320 and 2340 can be used to iteratively update a population model (at 2340). Depending on the implementation steps 2320 and 2340 can be used to either generate/derive a new populational model, or alternatively, to generate an updated population model that improves performance of the existing population model 2330 so that it provides improved estimates of glucose values for a subset of the population of particular users. Steps 2320 and 2340 of FIG. 23 are similar to steps as steps 2120 and 2130 of FIG. 21, and in some examples, steps 2320 and 2340 of FIG. 23 may be performed using a methodology such as that described above in accordance with the method 2100 of FIG. 21.

In accordance with the method 2300, at 2320, a subset of population data that is to be evaluated can be selected from a set of population data 2310. The subset of population data can be population data for a subset of the population of particular users. For instance, in one implementation, the population data for the subset of users can be selected as described above with reference to step 2120 of FIG. 21 (e.g., selected population data for a subset of users that share one or more common user characteristics and/or common therapy criteria).

At 2340, population learning can then be performed, where an existing population model 2330 can be trained or retrained, based on the subset of the population data that was selected at 2320, to generate a new updated population model 2345.

Steps 2350, 2370, 2380, 2384, 2386, 2390 of FIG. 23 are the same or similar to corresponding steps 2200, 2240, 2250, 2270, 2280, 2290 of FIG. 22, and for sake of brevity the description of steps 2200, 2240, 2250, 2270, 2280, 2290 that have been described with reference to FIG. 22 will not be repeated in the description of FIG. 23. Although not illustrated, after step 2340, additional processing can be performed as described with respect to FIG. 21, such as, performing a plausibility testing process (at 2140 of FIG. 21) and/or performing a calibration point testing process (at 2150 of FIG. 21) to determine whether the new updated population model should be calibrated, and then repeating the processing described in FIG. 21 to iteratively update and refine a most recently updated version of the population model 2345.

In accordance with the disclosed examples, technologies are provided for confirming that one or more personalized models satisfy any number of performance criteria used to analyze performance of that personalized model. Each personalized model that satisfies the performance criteria can then be added to a group or pool for further evaluation. One of the personalized models from the pool may then be selected based on selection criteria.

The selection criteria may vary depending on the implementation. In some examples, the personalized model having optimal/minimal sensor wear duration can be selected from the pool. In another embodiment, the personalized model having an optimal/longest model longevity can be selected from the pool. In still another embodiment, a combination of these selection criteria can be employed such that the personalized model having an optimized balance between a relatively short sensor wear period and a relatively long model longevity can be selected from the pool.

In some examples, the personalized model that is selected from the pool can be a different personalized model for each particular user (e.g., the personalized model having an optimal sensor wear duration or period that has been optimized for a particular user or patient). In other examples, the model that is selected from the pool can be a population model that provides the optimal sensor wear duration or period that is optimized for multiple users on average, and/or optimal model longevity that is optimized for multiple users on average. In these examples, the same time window and personalized learning process can be tested over multiple iterations for multiple users, and performance of the personalized model can be assessed across the multiple users such that a population model is selected that provides the optimal sensor wear duration or period that is optimized for multiple users on average, and/or optimal model longevity that is optimized for multiple users on average.

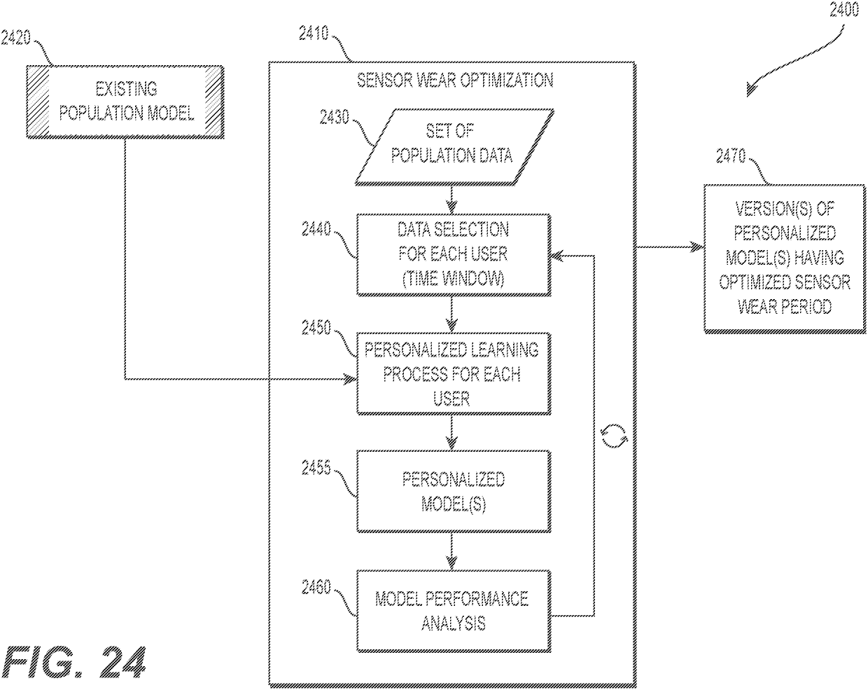

FIGS. 24-26 are flow charts that illustrate examples of methods in accordance with the disclosed examples that can be used to optimize sensor wear period and/or longevity of a personalized model used for estimating glucose values of a particular user as described herein. With respect to FIGS. 24-26, the steps of each method shown are not necessarily limiting. Steps can be added, omitted, and/or performed simultaneously without departing from the scope of the appended claims. Each method may include any number of additional or alternative tasks, and the tasks shown need not be performed in the illustrated order. Each method may be incorporated into a more comprehensive procedure or process having additional functionality not described in detail herein. Moreover, one or more of the tasks shown could potentially be omitted from an embodiment of each method as long as the intended overall functionality remains intact. Further, each method is computer-implemented in that various tasks or steps that are performed in connection with each method may be performed by software, hardware, firmware, or any combination thereof. For illustrative purposes, the following description of each method may refer to elements described herein. In certain examples, some or all steps of this process, and/or substantially equivalent steps, are performed by execution of processor-readable instructions stored or included on a processor-readable medium. For instance, in the description of FIGS. 24-26 that follows, it should be appreciated that steps of the methods refer to processor(s) or processing system(s) executing instructions to perform those various acts, tasks or steps. Depending on the implementation, some of the processor(s) or processing system(s) can be centrally located, or distributed among a number of systems that work together.

As described above, an existing population model may be used to initialize the creation of a personalized model that is tailored to each user, for example, through a variety of machine learning or statistical techniques (e.g., transfer learning, ensemble learning, etc.). It would be desirable to determine a minimum time window of sensor data that can be used to personalize a population model for each user, while still achieving satisfactory performance. This can reduce the amount of sensor glucose data need for consideration and evaluation during a personalized learning process, and thus reduce the duration of a sensor wear period that a particular user needs to wear a glucose sensor to acquire sensor data during personalized learning.

In accordance with the some of the disclosed examples, technologies are provided for confirming that one or more personalized models satisfy any number of performance criteria used to analyze performance of that personalized model. Each personalized model that satisfies the performance criteria can then be added to a group or pool for further evaluation. One of the personalized models from the pool may then be selected based on selection criteria. The selection criteria may vary depending on the implementation, and in some examples, the personalized model having optimal/minimal sensor wear duration can be selected from the pool. In some examples, the personalized model that is selected from the pool can be a different personalized model for each particular user (e.g., the personalized model having an optimal sensor wear duration or period that has been optimized for a particular user or patient). In other examples, the model that is selected from the pool can be a population model that provides the optimal sensor wear duration or period that is optimized for multiple users on average.

FIG. 24 is a flow chart of a method 2400 for optimizing a sensor wear period in accordance with the disclosed examples. As will be explained below, in accordance with method 2400, data for one or more users can be processed to determine or identify a personalized model having an optimal sensor wear period for each user.

A set of population data 2430 is provided that can include data for multiple different users. In some examples, the data that is included in the set of population data 2430 can include any of the data that is described above with reference to FIG. 21, and for sake of brevity the description of the set of population data 2110 from FIG. 21 will not be repeated here. As one non-limiting example, data that is included in the set of population data 2430 can include historical data that is collected over time such as: data from a glucose monitoring device associated with the particular user (e.g., sensor glucose data), data regarding consumption of macronutrients by the particular user, contextual information for each user such as contextual activity data associated with the particular user, etc.

At 2440, a data selection process can select a time window of data for each user from the set of population data 2430. Each time window of data can be, for example, a subset of data for that user that spans or is time-bound by a certain duration of time corresponding to a sensor wear period (e.g., 5 days of data for the particular user, 7 days of data for the particular user, 14 days of data for the particular user, etc.). In other words, the duration of the time window corresponds to a sensor wear period that a user wears a sensor to acquire data that is used to calibrate the personalized model of that user. Data of each user may be filtered such that only data that is within a specified time window for each user is selected for training the existing population model 2420 (at 2450), and any remaining data that is not selected can be reserved for other purposes, such as model validation.

Depending on the implementation, the duration of the time window can vary. For instance, in some examples, the time window of data for each user (e.g., that is needed to personalize a population model) can be determined based on factors such as accuracy requirements, business requirements, etc.

At 2450, the time window of data that was selected for each user can be applied to the existing population model 2420 to generate a personalized model that is personalized for the particular user. To explain further, the time window of data that was selected for each user can be used as training data during a personalized learning process at 2450 to adapt the population model 2420 resulting in a personalized model 2455 for each user. In other words, the personalized learning process adapts (e.g., calibrates) the population model 2420 using the time window of data that was selected for that user (at 2440) to generate or derive a personalized model 2455 that is personalized for each user.

The personalized learning process at 2450 can vary depending on the implementation. For example, in some examples, any of the personalized learning techniques described herein can be implemented at 2450, such as those described with reference to FIGS. 14-20. For instance, as an example of one non-limiting implementation, the personalized learning process at 2450 can employ transfer learning, whereas in another non-limiting implementation, the personalized learning process at 2450 can employ ensemble learning as described above. In some examples, on any given iteration of the method 2400, the personalized learning process that is applied at 2450 can be the same for all users (e.g., the same modeling approach is used for all users).

At 2460, performance of each of the personalized models (that were derived at 2450) can be analyzed to determine whether the personalized model satisfies performance criteria that are indicative of performance of that personalized model. This can be done, for example, by evaluating each personalized model using one or more performance criteria that are indicative of performance of that personalized model (e.g., that reflect relative performance of that personalized model), and determining whether that personalized model satisfies those performance criteria. In some examples, validation data for each individual user can be applied to each personalized model to conduct a performance analysis of that personalized model.

The performance criteria that are used to evaluate each personalized model at 2460 can vary depending on the implementation and the specific end application goals. The performance criteria that can be used to evaluate each personalized model at 2460 can be performance metrics, thresholds, and/or constraints that are required to be satisfied by the personalized model to satisfy specified performance requirements. Some non-limiting examples of the performance criteria that can be used to evaluate each personalized model at 2460 can include, but are not limited to: root mean squared error, mean relative difference, detectability of glucose excursions, accuracy based on time of day, etc.

If a personalized model does not satisfy one or more of the performance criteria (e.g., does not meet the specified performance requirements), that instance of the personalized model can be disregarded. In some examples, if a personalized model satisfies performance requirements specified by each of the performance criteria (e.g., meets the specified performance requirements), that instance of the personalized model can be added to a set or pool of the personalized models for that user that have been determined to satisfy each of the performance criteria (that have met the specified performance requirements). The personalized models in the set are candidates for selection as the personalized model that has the optimal sensor wear period. Ultimately one of the personalized models in that pool that has the optimal sensor wear period can be selected.

In some examples, the personalized model that is selected from the pool can be a different personalized model for each particular user (e.g., the personalized model having an optimal sensor wear duration or period that has been optimized for a particular user or patient)-. In some examples, the personalized model that has the optimal sensor wear period is the personalized model that requires a minimum wear duration to acquire data needed to satisfy any required performance criteria.

In other examples, the model that is selected from the pool can be a population model that provides the optimal sensor wear duration or period that is optimized for multiple users on average. In these examples, a common personalized learning process can be utilized that provides the best overall performance across a set of population data. The same personalized learning process can be evaluated for each user. Even though each user would have a unique personalized model, the process of generating it would be identical.

Following performance analysis at 2460, the method 2400 loops back to 2440 where another iteration of steps 2440, 2450, 2460 can be performed. During each iteration, a different time window of data can be selected (at 2440) such that a different personalized learning process can be performed (at 2450). Steps 2440, 2450, 2460 can repeat/loop to iteratively re-evaluate the personalized models that are derived for each user during each iteration of steps 2440, 2450, 2460. The steps of selecting at time window for each user (at 2440), generating a personalized model for each user (at 2450) and analyzing performance of each personalized model (at 260) can be repeated over any number of iterations to determine a set of personalized models that satisfy the one or more performance criteria. Steps 2440, 2450, 2460 can repeat/loop until a version 2470 of the personalized model for each user is identified/determined that satisfies/achieves one or more performance criteria that are used to evaluate each personalized model at 2460, while also exhibiting an optimal sensor wear period. In some examples, for each user, one of the personalized models that is determined to have an optimal sensor wear period can be selected from the set of the personalized models (that have been determined to satisfy each of the performance criteria) as the personalized model to be deployed as the personalized model for the particular user (e.g., deployed to a computer associated with the particular user, such as a mobile device, an insulin therapy device, a cloud-based server system, etc.).

In some examples, the sensor wear period indicates a minimum duration (e.g., number of days/hours/minutes) of sensor glucose data that is required to satisfy the performance analysis (at 2460). For example, in some examples, steps 2440, 2450, 2460 can repeat/loop until a version 2470 of the personalized model (for each user) is identified/determined that satisfies performance criteria (at 2460) and has a sensor wear period that has the minimum duration or “optimal sensor wear period.” The personalized model having the optimal sensor wear period can then be deployed to a device, system or other computer that is associated with the user, where it can be used as described herein to estimate glucose values of the user without the need for a glucose monitoring device (e.g., sensor).

Although not illustrated in FIG. 24, in accordance with some implementations, the step of repeating (as indicated, for example, by the feedback loop between 2460 and 2440) may include repetition of all steps for a subset of users (e.g., each user of a plurality of users). In those embodiments, the steps of selecting (at 2440), applying a personalized learning process for each user (at 2450), and analyzing performance to determine the optimized sensor wear period (at 2460) can be repeated over a number of iterations to determine a set of personalized models, for each user of the plurality of users, that satisfy each of the performance criteria. In those implementations, an analysis can be performed across all of the personalized models (derived for each user of the plurality of users and that satisfy the performance criteria) to determine which one has the optimal sensor wear period. In some implementations, the durations of the optimal sensor wear period can be evaluated using any type of statistical measure, such as an average or median, to determine which one has the optimal sensor wear period.

In some examples, the personalized model that is selected from the pool can be a different personalized model for each particular user. In other examples, the model that is selected from the pool can be a population model that provides the optimal sensor wear duration or period that is optimized for multiple users on average (e.g., as described above). In these examples, the same time window and personalized learning process can be tested over multiple iterations for multiple users, and performance of the personalized model can be assessed across the multiple users such that a population model is selected that provides the optimal sensor wear duration or period that is optimized for multiple users on average.

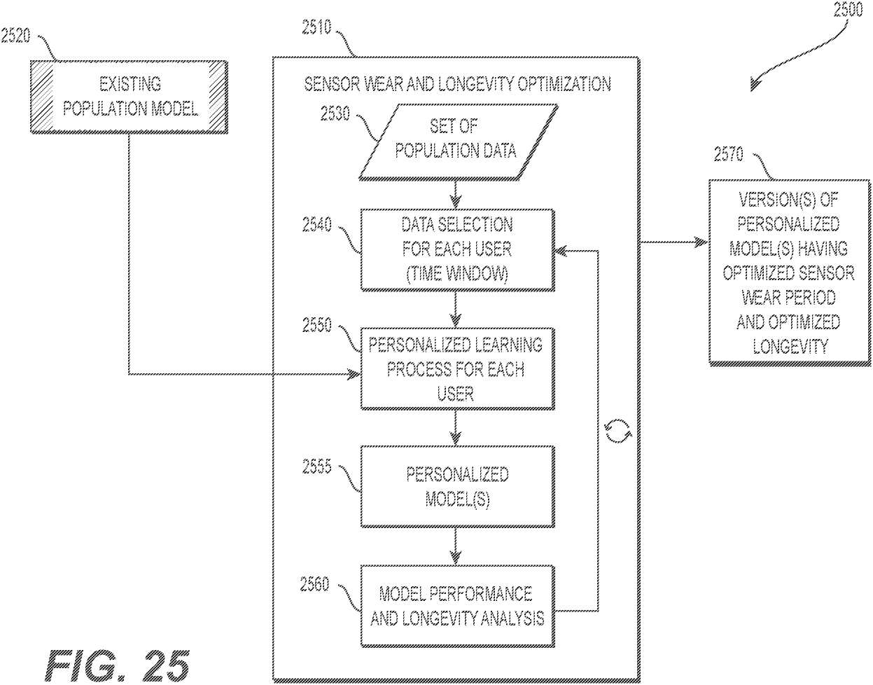

In some examples, selection criteria used to select one of the personalized models from the set of the personalized models (that have been determined to satisfy each of the performance criteria) can also, or alternatively, include model longevity. An example will now be described with reference to FIG. 25, where the selection criteria that are evaluated to select one of the personalized models as the model to be deployed can also include model longevity in addition to sensor wear period. In this embodiment, the personalized model that is selected for deployment can be optimized to have a relatively short sensor wear period and a relatively long model longevity. For example, in some cases, a personalized model that performs well after calibrating with three days of glucose sensor data may be considered to have an acceptable sensor wear period (e.g., an acceptable number of wear days), but may or may not perform well beyond a certain time (e.g., two weeks) after the user stops wearing the sensor. An optimal personalization approach can result in the selection of a personalized model having an optimized balance between a relatively short sensor wear period and a relatively long model longevity.

In accordance with the some of the disclosed examples, technologies are provided for confirming that one or more personalized models satisfy any number of performance criteria used to analyze performance of that personalized model. -Each personalized model that satisfies the performance criteria can then be added to a group or pool for further evaluation. One of the personalized models from the pool may then be selected based on selection criteria. The selection criteria may vary depending on the implementation. In some examples, the personalized model having an optimal/longest model longevity can be selected from the pool. In another embodiment, the personalized model having an optimized balance between a relatively short sensor wear period and a relatively long model longevity can be selected from the pool.

FIG. 25 is a flow chart of a method 2500 for optimizing sensor wear period and model longevity of a personalized model used for estimating glucose values of a particular user in accordance with the disclosed examples. The method 2500 includes many of the same steps 2540, 2550 as method 2400 of FIG. 24, and for sake of brevity, the description of those steps 2440, 2450 from FIG. 24 will not be repeated. Likewise, the set of population data 2530 utilized in the method 2500 is described above with reference to set of population data 2430 of FIG. 24, and for sake of brevity the description of set of population data 2430 will not be repeated here.

The method 2500 differs from method 2400 of FIG. 24 in that the performance analysis performed at 2560 also evaluates or assesses model longevity in addition to sensor wear period. In other words, in this embodiment, the selection criteria at 2560 can also include model longevity in addition to sensor wear period. In this regard, the model longevity can refer to a duration of time, post calibration (e.g., using a glucose sensor and contextual data), that a personalized model continues to perform within specified performance criteria when a glucose sensor is no longer available. As a non-limiting example, in some applications, a desired model longevity can be seven days, meaning that the personalized model needs to be calibrated with a glucose sensor every seven days. As another non-limiting example, in other applications, the desired model longevity can be 30 days, meaning that the personalized model needs to be calibrated with a glucose sensor every 30 days.

In some examples, model longevity of each personalized model can be evaluated or assessed at 2560 by determining metrics (or a series of metrics) that measure model longevity of that personalized model over time, and then comparing the model longevity of each personalized model to determine which personalized model has optimal model longevity (e.g., a model longevity of maximum duration). As described above with reference to FIG. 24, examples of these metrics can include, for example, root mean squared error, mean relative difference, detectability of glucose excursions, accuracy based on time of day, etc. Alternatively, or additionally, in some examples, model longevity of each personalized model can be evaluated or assessed at 2560 by evaluating model performance drift for any of the metrics over time. Alternatively, or additionally, in some examples, model longevity of each personalized model can be evaluated or assessed at 2560 by evaluating excursion detection for any of the metrics over time.

In some examples, steps 2540, 2550, 2560 can repeat/loop until a version 2570 of the personalized model is identified/determined that has a specified model longevity, or that has a model longevity that has the maximum duration (e.g., longest model longevity). In some examples, for each user, one of the personalized models that is determined to have the optimized balance between a relatively short sensor wear period and a relatively long model longevity can be selected (from the set of the personalized models that have been determined to satisfy each of the performance criteria). In some examples, the personalized model that is selected from the pool can be a different personalized model for each particular user (e.g., the personalized model having an optimal/longest model longevity that has been optimized for a particular user or patient). A personalized model for each particular user can be deployed for use by that particular user.

In other examples, the model that is selected from the pool can be a population model that provides the optimal sensor wear duration or period that is optimized for multiple users on average, and optimal model longevity that is optimized for multiple users on average. In these examples, the same time window and personalized learning process can be tested over multiple iterations for multiple users, and performance of the personalized model can be assessed across the multiple users such that a population model is selected that provides the optimal sensor wear duration or period that is optimized for multiple users on average, and/or optimal model longevity that is optimized for multiple users on average.

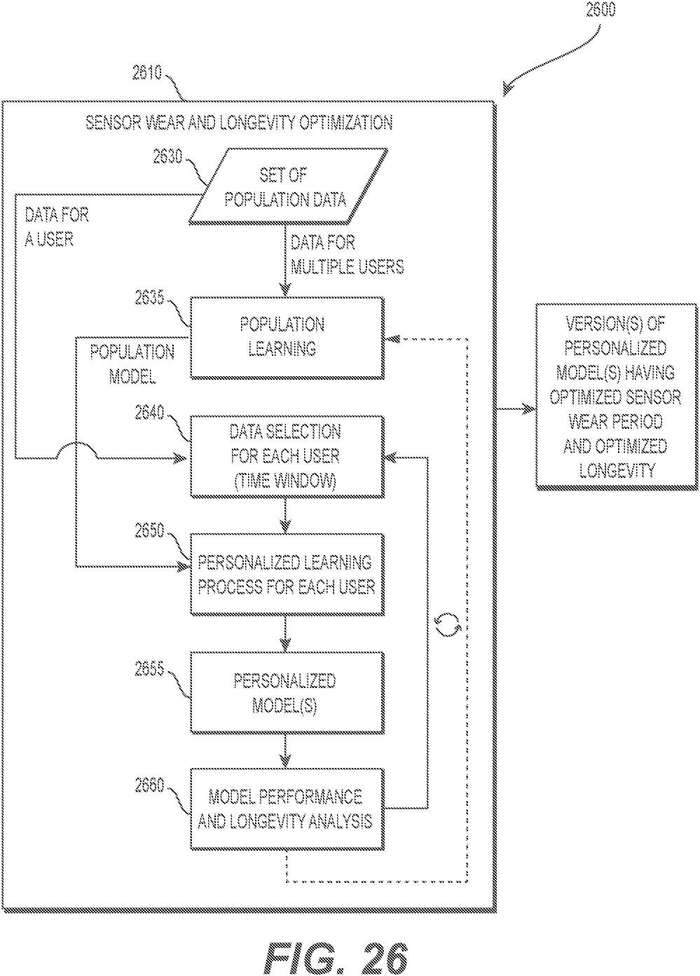

In some examples, such as those described with reference to FIGS. 24 and 25, an existing population model may already exist that can be adapted via personalized learning to derive a version of each personalized model that provides an optimal sensor wear period and/or model longevity. However, the population model that is used to derive the personalized models directly impacts the performance of the derived, personalized models, and for that reason, it may be desirable to adjust the population model to refine and optimize it as the personalized models change. As such, in other examples, a population learning step can also be included or integrated into the optimization workflow/pipeline, such that the overall learning approach is adjusted based on the results of the performance and longevity analysis, as will now be described with reference to FIG. 26. This can allow for a set of population modeling approaches, and their derived population models, to be assessed for their impact on personalized learning.

Although not illustrated in FIG. 25, in accordance with some implementations, the steps of repeating (as indicated, for example, by the feedback loop between 2560 and 2540) may include repetition of steps 2540, 2550 and 2560 for a subset of users (e.g., each user of a plurality of users). In those embodiments, the steps of selecting (at 2540), applying a personalized learning process for each user (at 2550), and analyzing performance to determine the optimized sensor wear period and model longevity (at 2560) can be repeated over a number of iterations to determine a set of personalized models, for each user of the plurality of users, that satisfy each of the performance criteria. In those implementations, an analysis can be performed across all of the personalized models (derived for each user of the plurality of users and that satisfy the performance criteria) to determine which one has the optimal sensor wear period and/or model longevity. In some implementations, the durations of the optimal sensor wear period and/or model longevity can be evaluated using any type of statistical measure, such as an average or median, to determine which one has the optimal sensor wear period and/or the optimal model longevity.

FIG. 26 is a flow chart of another method 2600 for optimizing sensor wear period and longevity of a personalized model used for estimating glucose values of a particular user in accordance with the disclosed examples. The method 2600 includes many of the same steps 2640, 2650, 2655, 2660 as method 2500 of FIG. 25, and for sake of brevity, the description of those steps 2540, 2550, 2555, 2560 from FIG. 25 will not be repeated.

The method 2600 differs from method 2500 of FIG. 25 in that a population learning step is performed at 2635 to derive a population model from the set of population data 2430 prior to selecting the time window of data for a particular user (at 2640). In most cases, the data that is selected in the data selection step (2640) is selected from data for a particular user, whereas the data that is used by the population learning step (at 2635) is data for multiple users (e.g., the data used for population learning step and personalized learning step are mutually exclusive). The population model that is derived (at 2635) can then be utilized during the personalized learning process (at 2650) to derive personalized models 2655. After the personalized models 2655 are analyzed at 2660, another iteration of steps 2635, 2640, 2650 can be performed.

As illustrated by the dashed-line arrow connecting 2660 to 2635, in some examples, prior to each iteration of repeating the steps of selecting (at 2640), generating (at 2650) and analyzing (at 2660), the population model can be updated to derive a new updated population model. During each iteration of the method 2600, the population model that is derived during the population learning step at 2635 can be adjusted to refine and optimize it as the set of population data 2630 changes. In some implementations, during each iteration of updating the population model (at 2635) a new subset of the population data 2630 can be selected, and/or new or different machine learning models can be selected to derive the new updated population model (e.g., new or different machine learning models that are used to derive the new updated population model by applying the new subset of the population data 2630 to the machine learning models). In other words, depending on the implementation, during each iteration of steps 2635, 2640, 2650, 2660, the population learning step 2635 can be varied, for example, by selecting a new or different subset of the population data 2630 to derive the population model, selecting any combination of different machine learning models to be applied to the selected population data 2630 to derive the population model, changing the parameter initialization of the population models, and/or applying different optimization methods to the population model.

Although not illustrated in FIG. 26, in accordance with some implementations, the steps of repeating (as indicated, for example, by the feedback loop between 2660 and 2635 and/or by the feedback loop between 2660 and 2640) may include repetition of those steps for a subset of users (e.g., each user of a plurality of users). In those embodiments, the steps of selecting (at 2640), applying a personalized learning process for each user (at 2650), and analyzing performance to determine the optimized sensor wear period and model longevity (at 2660) can be repeated over a number of iterations to determine a set of personalized models, for each user of the plurality of users, that satisfy each of the performance criteria. In those implementations, an analysis can be performed across all of the personalized models (derived for each user of the plurality of users and that satisfy the performance criteria) to determine which one has the optimal sensor wear period and/or model longevity. In some implementations, the durations of the optimal sensor wear period and/or model longevity can be evaluated using any type of statistical measure, such as an average or median, to determine which one has the optimal sensor wear period and/or the optimal model longevity.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application.

What is claimed is:

1. A method, comprising:
selecting, from a set of population data, a time window of data for a particular user that comprises: a subset of data for the particular user to be used for training a population model, wherein the time window of data is recorded over a period that corresponds to a sensor wear period;
applying the subset of data to the population model to generate a personalized model for estimating glucose values that is personalized for the particular user;
analyzing performance of the personalized model to determine whether the personalized model satisfies performance criteria that are indicative of performance of that personalized model;
repeating the steps of selecting, applying and analyzing over a number of iterations to determine a set of personalized models that satisfy each of the performance criteria;
selecting, from the set of the personalized models that have been determined to satisfy each of the performance criteria, one of the personalized models that is determined to have an optimal sensor wear period as the personalized model to be deployed for the particular user; and
deploying the personalized model having the optimal sensor wear period as the personalized model for the particular user.

2. The method of claim 1, wherein the time window that is selected during each iteration of the method is adjusted to encompass a different subset of data for the particular user to be used for training the population model, and wherein repeating comprises:
during each iteration of the selecting step: selecting a different time window of data for the particular user; and
during each iteration of the applying step: applying a subset of data from the different time window of data that was selected during that iteration to the population model to generate a different personalized model that is personalized for the particular user.

3. The method of claim 1, wherein applying comprises:
training the population model based on the subset of data to adapt the population model and derive the personalized model that is personalized for the particular user.

4. The method of claim 1, wherein analyzing comprises:
for each of the performance criteria that are indicative of performance of that personalized model: determining whether the personalized model satisfies performance requirements specified by that performance criteria; and
adding the personalized model to the set of the personalized models that have been determined to satisfy each of the performance criteria when the personalized model satisfies performance requirements specified by each of the performance criteria.

5. The method of claim 4, wherein the performance criteria used to evaluate each personalized model comprise:
performance metrics that are required to be satisfied by the personalized model, performance thresholds that are required to be satisfied by the personalized model, or performance that are required to be satisfied by the personalized model.

6. The method of claim 1, wherein the set of population data comprises data for each user comprises:
historical data that is collected over time comprising one or more of:
data from a glucose monitoring device associated with the particular user;
data regarding consumption of macronutrients by the particular user; and
contextual information comprising contextual activity data associated with the particular user.

7. The method of claim 1, wherein each sensor wear period is a duration that a user is required to wear a sensor for in order to acquire a sufficient amount of data needed to calibrate a personalized model of that user such that it satisfies any required performance criteria, and
wherein the personalized model that has the optimal sensor wear period is the personalized model that requires a minimum wear duration to acquire data needed to satisfy any required performance criteria.

8. The method of claim 1, further comprising:
determining metrics that measure model longevity of each personalized model over time;
comparing the model longevity of each personalized model to determine which personalized model has an optimal model longevity of maximum duration;
from the set of the personalized models that have been determined to satisfy each of the performance criteria: selecting one of the personalized models that is determined to have an optimized balance between the sensor wear period and model longevity as the personalized model to be deployed to the particular user; and
wherein deploying comprises:
deploying the personalized model having the optimized balance between the sensor wear period and model longevity as the personalized model for the particular user.

9. The method of claim 8, wherein the model longevity is a duration of time after calibration that a personalized model continues to perform within specified performance criteria when a glucose sensor is no longer available.

10. The method of claim 1, further comprising:
deriving the population model from the set of population data prior to selecting the time window of data.

11. The method of claim 10, further comprising:
prior to each iteration of repeating the steps of selecting, applying and analyzing: updating the population model to derive a new updated population model.

12. The method of claim 9, wherein updating the population model comprises:
during each iteration of updating the population model performing at least one of:
selecting a new subset of the population data to derive the new updated population model; and
selecting one or more machine learning models that are used to derive the new updated population model by applying the new subset of the population data to the one or more selected machine learning models.

13. The method of claim 1, wherein repeating comprises:
for each user of a plurality of users:
repeating the steps of selecting, applying, and analyzing over a number of iterations to determine a set of personalized models, for each user of the plurality of users, that satisfy each of the performance criteria; and
wherein selecting one of the personalized models comprises:
performing a statistical analysis to determine which one of the set of personalized models that satisfy each of the performance criteria for each of the plurality of users has the optimal sensor wear period; and
selecting the one of the set of personalized models that satisfies each of the performance criteria for each of the plurality of users as the personalized model that has the optimal sensor wear period.

14. A system, comprising:
one or more hardware-based processors configured by machine-readable instructions to
select, from a set of population data, a time window of data for a particular user that comprises: a subset of data for the particular user to be used for training a population model, wherein the time window of data is recorded over a period that corresponds to a sensor wear period;
apply the subset of data to the population model to generate a personalized model for estimating glucose values that is personalized for the particular user;
analyze performance of the personalized model to determine whether the personalized model satisfies performance criteria that are indicative of performance of that personalized model;
repeat the steps of selecting, applying and analyzing over a number of iterations to determine a set of personalized models that satisfy each of the performance criteria;
select, from the set of the personalized models that have been determined to satisfy each of the performance criteria, one of the personalized models that is determined to have an optimal sensor wear period as the personalized model to be deployed for the particular user; and
deploy the personalized model having the optimal sensor wear period as the personalized model for the particular user.

15. The system of claim 14, wherein the time window that is selected during each iteration of the method is adjusted to encompass a different subset of data for the particular user to be used for training the population model, and wherein repeating comprises:
during each iteration of the selecting step: selecting a different time window of data for the particular user; and
during each iteration of the applying step: applying a subset of data from the different time window of data that was selected during that iteration to the population model to generate a different personalized model that is personalized for the particular user.

16. The system of claim 14, wherein analyzing comprises:
for each of the performance criteria that are indicative of performance of that personalized model: determining whether the personalized model satisfies performance requirements specified by that performance criteria; and
adding the personalized model to the set of the personalized models that have been determined to satisfy each of the performance criteria when the personalized model satisfies performance requirements specified by each of the performance criteria.

17. The system of claim 16, wherein the performance criteria used to evaluate each personalized model comprise:
performance metrics that are required to be satisfied by the personalized model, performance thresholds that are required to be satisfied by the personalized model, or performance that are required to be satisfied by the personalized model.

18. The system of claim 14, wherein each sensor wear period is a duration that a user is required to wear a sensor for in order to acquire a sufficient amount of data needed to calibrate a personalized model of that user such that it satisfies any required performance criteria, and
wherein the personalized model that has the optimal sensor wear period is the personalized model that requires a minimum wear duration to acquire data needed to satisfy any required performance criteria.

19. The system of claim 14, further comprising:
determining metrics that measure model longevity of each personalized model over time;
comparing the model longevity of each personalized model to determine which personalized model has an optimal model longevity of maximum duration;
from the set of the personalized models that have been determined to satisfy each of the performance criteria: selecting one of the personalized models that is determined to have an optimized balance between the sensor wear period and model longevity as the personalized model to be deployed to the particular user; and
wherein deploying comprises:
deploying the personalized model having the optimized balance between the sensor wear period and model longevity as the personalized model for the particular user.

20. At least one non-transient computer-readable medium having instructions stored thereon that are configurable to cause at least one processor to perform a method, the method comprising:
selecting, from a set of population data, a time window of data for a particular user that comprises: a subset of data for the particular user to be used for training a population model, wherein the time window of data is recorded over a period that corresponds to a sensor wear period;
applying the subset of data to the population model to generate a personalized model for estimating glucose values that is personalized for the particular user;
analyzing performance of the personalized model to determine whether the personalized model satisfies performance criteria that are indicative of performance of that personalized model;
repeating the steps of selecting, applying and analyzing over a number of iterations to determine a set of personalized models that satisfy each of the performance criteria;
selecting, from the set of the personalized models that have been determined to satisfy each of the performance criteria, one of the personalized models that is determined to have an optimal sensor wear period as the personalized model to be deployed for the particular user; and
deploying the personalized model having the optimal sensor wear period as the personalized model for the particular user.
